(19) United States
(12) Patent Application Publication
(10) Pub. No.: US 2022/0089757 A1
(43) Pub. Date: Mar. 24, 2022

(54) ANTI-CD137L ANTIBODIES AND METHODS OF USING SAME

(71) Applicant: ADAGENE PTE. LTD., Singapore (SG)

(72) Inventors: Peter Peizhi LUO, San Mateo, CA (US); Fangyong DU, Suzhou, Jiangsu (CN); Guizhong LIU, Suzhou, Jiangsu (CN)

(73) Assignee: ADAGENE PTE. LTD., Singapore (SG)

(21) Appl. No.: 17/612,878

(22) PCT Filed: Jun. 4, 2020
(86) PCT No.: PCT/CN2020/094371

§ 371 Date: Nov. 19, 2021

(30) Foreign Application Priority Data
Jun. 5, 2019 (CN) PCT/CN2019/090091

(51) Int. Cl. C07K 16/28; G01N 33/574; A61P 35/00; A61K 39/00

(52) U.S. Cl. CPC C07K 16/2875; G01N 33/57492; C07K 2317/55; A61K 2039/505; C07K 2317/34; A61P 35/00

(57) ABSTRACT

Provided herein are antibodies that bind to an intracellular/transmembrane portion of human CD137L, as well as methods relating to the use of these antibodies for detecting CD137L expression in a sample and the detection of CD137L expression as a biomaker for cancer treatment.

### CROSS REFERENCE TO RELATED APPLICATIONS

This application claims the priority benefit of International Application No. PCT/CN2019/090091, filed on Jun. 5, 2019, which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 695402000841SEQLIST.TXT, date recorded: Jun. 2, 2020, size: 176 KB).

### FIELD OF THE INVENTION

The present disclosure relates to antibodies that bind CD137 Ligand (CD137L), as well as the use of such antibodies, e.g., to detect CD137L expression as a biomarker.

### BACKGROUND

Activation of T cells plays a central role in antitumor immunity. Two key signals are required to activate naïve T cells. Signal one is provided through the T-cell receptor (TCR), while signal two is that of co-stimulation. The CD28:B7 molecules are some of the best-studied costimulatory pathways, thought to be the main mechanism through which primary T cell stimulation occurs. However, a number of other molecules have been identified which serve to amplify and diversify the T cell response following initial T cell activation. These include CD137:CD137 ligand (CD137L) molecules, also known as 4-1BB: 4-1BB ligand (4-1BBL). CD137:CD137L are members of the Tumor Necrosis Factor (TNF) Receptor (TNFR):TNF ligand family, which are expressed on T cells and antigen-presenting cells (APCs), respectively. Therapies targeting the CD137:CD137L signaling pathway have been shown to have antitumor effects in a number of model systems, and agonistic anti-CD137 antibodies have also entered clinical development (Yonezawa et al. Clin. Cancer Res. 2015 Jul. 15; 21(14):3113-20; Tolcher et al. Clin Cancer Res. 2017 Sep. 15; 23(18):5349-5357). Also, CD137 ligand cross-links its receptor, CD137, which is expressed on activated T cells, and costimulates T cell activities. CD137 ligand can also be expressed as a transmembrane protein on the cell surface and transmit signals into the cells on which it is expressed (reverse signaling) (Herbert Schwarz et al. J. Leukoc. Biol. 89: 21-29; 2011).

Previous studies indicated that CD137L gene delivery into multiple mouse tumor models can enable the host mice to develop long-term immunity against wild-type tumors (Melero et al. Eur. J. Immunol. 1998 March; 28(3):1116-21), prevent tumor formation or induce tumor regression from the transfectants (Guinn et al. J Immunol. 1999 Apr. 15; 162(8):5003-10; Xiang Cancer Biother. Radiopharm. 1999 October; 14(5):353-61), or improve host survival (Martinet et al. J Natl Cancer Inst. 2000 Jun. 7; 92(11):931-6). These results suggest that CD137L expression may modify the tumor cells for whole cell vaccination by improving their ability to act as APCs for their tumor antigens, with the costimulatory CD137L molecules providing an abundance of signal two.

It is thought that CD137L expression may be useful, e.g., in selecting patients for cancer treatments (such as treatment with an anti-CD137 antibody). However, this depends on the ability to detect CD137L in a sample (e.g., an IHC sample, such as from a patient) with high confidence, robustness, and accuracy. As such, there is a need for anti-CD137L antibodies that allow for highly sensitive and robust detection of CD137L expression in a sample.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

To meet the above and other needs, disclosed herein are anti-CD137L antibodies that specifically bind the intracellular and/or transmembrane domains of human CD137L. Since the extracellular domain of CD137L is known to be subject to cleavage/shedding in tissues, it is thought that detection of CD137L via the intracellular or transmembrane domain may provide a more accurate assessment of CD137L expression in a tissue, e.g., from a tumor. As such, the present disclosure provides anti-CD137L antibodies that bind the intracellular and/or transmembrane domains of human CD137L with high sensitivity and robustness, thereby enabling their use in detection of CD137L in a fixed sample (e.g., using IHC). Moreover, this detection may be used to inform treatment decisions, e.g., to determine whether a patient's tumor may be responsive to treatment with an anti-CD137 antibody.

Accordingly, in one aspect, provided herein are antibodies (e.g., isolated antibodies) or antigen-binding fragments thereof that bind to an intracellular or transmembrane region of human CD137 ligand (CD137L). In some embodiments, the antibody or antigen-binding fragment binds to a peptide comprising the amino acid sequence of MEYASDASLDPEAPWPPAPRARACRVLP (SEQ ID NO:1). In some embodiments, the antibody or antigen-binding fragment binds to a peptide comprising the amino acid sequence of MEYASDASLDPEAPWPPAPRARA (SEQ ID NO:2). In some embodiments, the antibody or antigen-binding fragment binds to a peptide comprising the amino acid sequence of MEYASDASLDPEAPWPPAPRARACRVLP (SEQ ID NO: 1) and binds to a peptide comprising the amino acid sequence of MEYASDASLDPEAPWPPAPRARA (SEQ ID NO:2). In some embodiments, the antibody or antigen-binding fragment binds to the extracellular domain of human CD137L. In some embodiments, the antibody or antigen-binding fragment binds to an intracellular domain or non-transmembrane region of human CD137L.

In some embodiments, the peptide comprising the amino acid sequence of MEYASDASLDPEAPWPPAPRARACRVLP (SEQ ID NO:1) is less than 50 amino acids in length. In some embodiments, the peptide comprising the amino acid sequence of MEYASDASLDPEAPWPPAPRARA (SEQ ID NO:2) is less than 50 amino acids in length. In some embodiments, the peptide comprising the amino acid sequence of MEYASDASLDPEAPWPPAPRARACRVLP (SEQ ID NO:1) is less than 50 amino acids in length, and the peptide comprising the amino acid sequence of MEYASDASLDPEAPWPPAPRARA (SEQ ID NO:2) is less than 50 amino acids in length. In some embodiments, the peptide comprising the amino acid sequence of MEYASDASLDPEAPWPPAPRARACRVLP (SEQ ID NO:1) does not comprise the extracellular domain of CD137L, or a portion thereof. In some embodiments, the peptide comprising the amino acid sequence of MEYASDASLDPEAPWPPAPRARA (SEQ ID NO:2) does not comprise the extracellular domain of CD137L, or a portion thereof. In some embodiments, the peptide comprising the amino acid sequence of MEYASDASLDPEAPWPPAPRARACRVLP (SEQ ID NO:1) and the peptide comprising the amino acid sequence of MEYASDASLDPEAPWPPAPRARA (SEQ ID NO:2) do not comprise the extracellular domain of CD137L, or a portion thereof. In some embodiments, wherein the antibody or antigen-binding fragment binds to a Chinese hamster ovary (CHO) cell that expresses human CD137L. In some embodiments, the antibody or antigen-binding fragment binds to human CD137L in a fixed human tissue sample, e.g., a formalin-fixed paraffin-embedded (FFPE) sample. In some embodiments, the sample is from human tonsil tissue or from human tumor tissue.

In some embodiments, the anti-CD137L antibody comprises a heavy chain variable region and a light chain variable region, wherein the heavy chain variable region comprises an HVR-H1 comprising an amino acid sequence according to Formula (I): LX₁TX₂GVGVX₃WI, wherein X₁ is S, N, or T; wherein X₂ is S, A, G, or T; and wherein X₃ is S, T, A, or G (SEQ ID NO:346); an HVR-H2 comprising an amino acid sequence according to Formula (II): X₁X₂X₃IDX₄X₅X₆X₇X₈YYX₉PSX₁₀KSX₁₁L, wherein X₁ is L or 1; wherein X₂ is A or G; wherein X₃ is L, V, or I; wherein X₄ is W, H, or Y: wherein X₅ is A or S; wherein X₆ is G or D: wherein X₇ is D, Y, A, or S; wherein X₅ is K or T: wherein X₉ is S or N; wherein X₁₀ is L or P; and wherein X₁₁ is R or H (SEQ ID NO:347); and an HVR-H3 comprising an amino acid sequence according to Formula (III): ARYGX₁X₂X₃YAX₄DY, wherein X₁ is Y, V, W, L, or G; wherein X₂ is S, G, R, D, or N; wherein X₃ is S, G, or N; and wherein X₄ is I, L, or M (SEQ ID NO:348); and/or wherein the light chain variable region comprises an HVR-L1 comprising an amino acid sequence according to Formula (IV): RX₁SQX₂X₃X₄X₅X₆X₇LA, wherein X₁ is A or T; wherein X₂ is S, T, or G; wherein X₃ is V or I; wherein X₄ is R, S, or H; wherein X₅ is G or N; wherein X₆ is S, N, or T; and wherein X₇ is Y or F (SEQ ID NO: 349); an HVR-L2 comprising an amino acid sequence according to Formula (V): X₁AX₂X₃LX₄SGV, wherein X₁ is A or D; wherein X₂ is S or F; wherein X₃ is T or S; and wherein X₄ is Q or E (SEQ ID NO:350); and an HVR-L3 comprising an amino acid sequence according to Formula (VI): YCQQYX₁SX₂WT, wherein X₁ is S, G, or N and wherein X₂ is L, Y, S, W or F (SEQ ID NO:351). In some embodiments, the antibody comprises a heavy chain variable region and a light chain variable region, wherein the heavy chain variable region comprises an HVR-H1 comprising the amino acid sequence of SEQ ID NO:4, an HVR-H2 comprising the amino acid sequence of SEQ ID NO:5, and an HVR-H3 comprising the amino acid sequence of SEQ ID NO:6, and/or the light chain variable region comprises an HVR-L1 comprising the amino acid sequence of SEQ ID NO:7, an HVR-L2 comprising the amino acid sequence of SEQ ID NO:8, and an HVR-L3 comprising the amino acid sequence of SEQ ID NO:9. In some embodiments, the antibody comprises a heavy chain variable region and a light chain variable region, wherein the heavy chain variable region comprises an HVR-H1 comprising the amino acid sequence of SEQ ID NO:10, an HVR-H2 comprising the amino acid sequence of SEQ ID NO:11, and an HVR-H3 comprising the amino acid sequence of SEQ ID NO:12, and/or the light chain variable region comprises an HVR-L1 comprising the amino acid sequence of SEQ ID NO:13, an HVR-L2 comprising the amino acid sequence of SEQ ID NO:14, and an HVR-L3 comprising the amino acid sequence of SEQ ID NO:15. In some embodiments, the antibody comprises a heavy chain variable region and a light chain variable region, wherein the heavy chain variable region comprises an HVR-H1 comprising the amino acid sequence of SEQ ID NO:16, an HVR-H2 comprising the amino acid sequence of SEQ ID NO:17, and an HVR-H3 comprising the amino acid sequence of SEQ ID NO:18, and/or the light chain variable region comprises an HVR-L1 comprising the amino acid sequence of SEQ ID NO:19, an HVR-L2 comprising the amino acid sequence of SEQ ID NO:20, and an HVR-L3 comprising the amino acid sequence of SEQ ID NO:21. In some embodiments, the antibody comprises a heavy chain variable region and a light chain variable region, wherein the heavy chain variable region comprises an HVR-H1 comprising the amino acid sequence of SEQ ID NO:22, an HVR-H2 comprising the amino acid sequence of SEQ ID NO:23, and an HVR-H3 comprising the amino acid sequence of SEQ ID NO:24, and/or the light chain variable region comprises an HVR-LI comprising the amino acid sequence of SEQ ID NO:25, an HVR-L2 comprising the amino acid sequence of SEQ ID NO:26, and an HVR-L3 comprising the amino acid sequence of SEQ ID NO:27. In some embodiments, the antibody comprises a heavy chain variable region and a light chain variable region, wherein the heavy chain variable region comprises an HVR-H1 comprising the amino acid sequence of SEQ ID NO:28, an HVR-H2 comprising the amino acid sequence of SEQ ID NO:29, and an HVR-H3 comprising the amino acid sequence of SEQ ID NO:30, and/or the light chain variable region comprises an HVR-L1 comprising the amino acid sequence of SEQ ID NO:31, an HVR-L2 comprising the amino acid sequence of SEQ ID NO:32, and an HVR-L3 comprising the amino acid sequence of SEQ ID NO:33. In some embodiments, the antibody comprises a heavy chain variable region and a light chain variable region, wherein the heavy chain variable region comprises an HVR-H1 comprising the amino acid sequence of SEQ ID NO:34, an HVR-H2 comprising the amino acid sequence of SEQ ID NO:35, and an HVR-H3 comprising the amino acid sequence of SEQ ID NO:36, and/or the light chain variable region comprises an HVR-L1 comprising the amino acid sequence of SEQ ID NO:37, an HVR-L2 comprising the amino acid sequence of SEQ ID NO:38, and an HVR-L3 comprising the amino acid sequence of SEQ ID NO:39. In some embodiments, the antibody comprises a heavy chain variable region and a light chain variable region, wherein the heavy chain variable region comprises an HVR-H1 comprising the amino acid sequence of SEQ ID NO:40, an HVR-H2 comprising the amino acid sequence of SEQ ID NO:41, and an HVR-H3 comprising the amino acid sequence of SEQ ID NO:42, and/or the light chain variable region comprises an HVR-L1 comprising the amino acid sequence of SEQ ID NO:43, an HVR-L2 comprising the amino acid sequence of SEQ ID NO:44, and an HVR-L3 comprising the amino acid sequence of SEQ ID NO:45. In some embodiments, the antibody comprises a heavy chain variable region and a light chain variable region, wherein the heavy chain variable region comprises an HVR-H1 comprising the amino acid sequence of SEQ ID NO:46, an HVR-H2 comprising the amino acid sequence of SEQ ID NO:47, and an HVR-H3 comprising the amino acid sequence of SEQ ID NO:48, and/or the light chain variable region comprises an HVR-L1 comprising the amino acid sequence of SEQ ID NO:49, an HVR-L2 comprising the amino acid sequence of SEQ ID NO:50, and an HVR-L3 comprising the amino acid sequence of SEQ ID NO:51. In some embodiments, the anti-CD137L antibody comprises an HVR-H1 comprising the amino acid sequence of SEQ ID NO:68, an HVR-H2 comprising the amino acid sequence of SEQ ID NO: 116, an HVR-H3 comprising the amino acid sequence of SEQ ID NO:164, an HVR-L1 comprising the amino acid sequence of SEQ ID NO:212, an HVR-L2 comprising the amino acid sequence of SEQ ID NO:228, and an HVR-L3 comprising the amino acid sequence of SEQ ID NO:232. In some embodiments, the anti-CDI 37L antibody comprises an HVR-H1 comprising the amino acid sequence of SEQ ID NO:69, an HVR-H2 comprising the amino acid sequence of SEQ ID NO:117, an HVR-H3 comprising the amino acid sequence of SEQ ID NO:165, an HVR-L1 comprising the amino acid sequence of SEQ ID NO:212, an HVR-L2 comprising the amino acid sequence of SEQ ID NO:228, and an HVR-L3 comprising the amino acid sequence of SEQ ID NO:233. In some embodiments, the anti-CD137L antibody comprises an HVR-H1 comprising the amino acid sequence of SEQ ID NO:70, an HVR-H2 comprising the amino acid sequence of SEQ ID NO:118, an HVR-H3 comprising the amino acid sequence of SEQ ID NO:166, an HVR-LI comprising the amino acid sequence of SEQ ID NO:213, an HVR-L2 comprising the amino acid sequence of SEQ ID NO:228, and an HVR-L3 comprising the amino acid sequence of SEQ ID NO:233. In some embodiments, the anti-CD137L antibody comprises an HVR-H1 comprising the amino acid sequence of SEQ ID NO:71, an HVR-H2 comprising the amino acid sequence of SEQ ID NO:119, an HVR-H3 comprising the amino acid sequence of SEQ ID NO:167, an HVR-L1 comprising the amino acid sequence of SEQ ID NO:212, an HVR-L2 comprising the amino acid sequence of SEQ ID NO:228, and an HVR-L3 comprising the amino acid sequence of SEQ ID NO:234. In some embodiments, the anti-CD137L antibody comprises an HVR-H1 comprising the amino acid sequence of SEQ ID NO:72, an HVR-H2 comprising the amino acid sequence of SEQ ID NO:120, an HVR-H3 comprising the amino acid sequence of SEQ ID NO:168, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:235. In some embodiments, the anti-CD137L antibody comprises an HVR-H1 comprising the amino acid sequence of SEQ ID NO:73, an HVR-H2 comprising the amino acid sequence of SEQ ID NO:121, an HVR-H3 comprising the amino acid sequence of SEQ ID NO:169, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:74, an HVR-H2 comprising the amino acid sequence of SEQ ID NO:122, an HVR-H3 comprising the amino acid sequence of SEQ ID NO:170, an HVR-LI comprising the amino acid sequence of SEQ ID NO:214, an HVR-L2 comprising the amino acid sequence of SEQ ID NO:229, and an HVR-L3 comprising the amino acid sequence of SEQ ID NO:236. In some embodiments, the anti-CD137L antibody comprises an HVR-H1 comprising the amino acid sequence of SEQ ID NO:75, an HVR-H2 comprising the amino acid sequence of SEQ ID NO:123, an HVR-H3 comprising the amino acid sequence of SEQ ID NO:171, an HVR-L1 comprising the amino acid sequence of SEQ ID NO:215, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:76, an HVR-H2 comprising the amino acid sequence of SEQ ID NO:124, an HVR-H3 comprising the amino acid sequence of SEQ ID NO:172, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:235. In some embodiments, the anti-CD137L antibody comprises an HVR-H1 comprising the amino acid sequence of SEQ ID NO:77, an HVR-H2 comprising the amino acid sequence of SEQ ID NO:125, an HVR-H3 comprising the amino acid sequence of SEQ ID NO:173, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:235. In some embodiments, the anti-CD137L antibody comprises an HVR-H1 comprising the amino acid sequence of SEQ ID NO:78, an HVR-H2 comprising the amino acid sequence of SEQ ID NO:126, an HVR-H3 comprising the amino acid sequence of SEQ ID NO:174, an HVR-L1 comprising the amino acid sequence of SEQ ID NO:216, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:79, an HVR-H2 comprising the amino acid sequence of SEQ ID NO:127, an HVR-H3 comprising the amino acid sequence of SEQ ID NO:175, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:80, an HVR-H2 comprising the amino acid sequence of SEQ ID NO:128, an HVR-H3 comprising the amino acid sequence of SEQ ID NO:176, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:238. In some embodiments, the anti-CD137L antibody comprises an HVR-H1 comprising the amino acid sequence of SEQ ID NO:81, an HVR-H2 comprising the amino acid sequence of SEQ ID NO:129, an HVR-H3 comprising the amino acid sequence of SEQ ID NO:177, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:82, an HVR-H2 comprising the amino acid sequence of SEQ ID NO:130, an HVR-H3 comprising the amino acid sequence of SEQ ID NO:178, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:83, an HVR-H2 comprising the amino acid sequence of SEQ ID NO:131, an HVR-H3 comprising the amino acid sequence of SEQ ID NO:179, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:84, an HVR-H2 comprising the amino acid sequence of SEQ ID NO:132, an HVR-H3 comprising the amino acid sequence of SEQ ID NO:180, an HVR-L1 comprising the amino acid sequence of SEQ ID NO:218, an HVR-L2 comprising the amino acid sequence of SEQ ID NO:229, and an HVR-L3 comprising the amino acid sequence of SEQ ID NO:239. In some embodiments, the anti-CD137L antibody comprises an HVR-H1 comprising the amino acid sequence of SEQ ID NO:85, an HVR-H2 comprising the amino acid sequence of SEQ ID NO:133, an HVR-H3 comprising the amino acid sequence of SEQ ID NO:181, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:86, an HVR-H2 comprising the amino acid sequence of SEQ ID NO:134, an HVR-H3 comprising the amino acid sequence of SEQ ID NO:182, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:87, an HVR-H2 comprising the amino acid sequence of SEQ ID NO:135, an HVR-H3 comprising the amino acid sequence of SEQ ID NO:183, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:240. In some embodiments, the anti-CD137L antibody comprises an HVR-H1 comprising the amino acid sequence of SEQ ID NO:88, an HVR-H2 comprising the amino acid sequence of SEQ ID NO:136, an HVR-H3 comprising the amino acid sequence of SEQ ID NO:184, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:89, an HVR-H2 comprising the amino acid sequence of SEQ ID NO:137, an HVR-H3 comprising the amino acid sequence of SEQ ID NO:185, an HVR-L1 comprising the amino acid sequence of SEQ ID NO:219, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:90, an HVR-H2 comprising the amino acid sequence of SEQ ID NO:138, an HVR-H3 comprising the amino acid sequence of SEQ ID NO:186, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:91, an HVR-H2 comprising the amino acid sequence of SEQ ID NO:139, an HVR-H3 comprising the amino acid sequence of SEQ ID NO:187, an HVR-L1 comprising the amino acid sequence of SEQ ID NO:220, an HVR-L2 comprising the amino acid sequence of SEQ ID NO:228, and an HVR-L3 comprising the amino acid sequence of SEQ ID NO:233. In some embodiments, the anti-CD137L antibody comprises an HVR-H1 comprising the amino acid sequence of SEQ ID NO:92, an HVR-H2 comprising the amino acid sequence of SEQ ID NO:140, an HVR-H3 comprising the amino acid sequence of SEQ ID NO:188, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:93, an HVR-H2 comprising the amino acid sequence of SEQ ID NO:141, an HVR-H3 comprising the amino acid sequence of SEQ ID NO:189, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:94, an HVR-H2 comprising the amino acid sequence of SEQ ID NO:142, an HVR-H3 comprising the amino acid sequence of SEQ ID NO:190, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:95, an HVR-H2 comprising the amino acid sequence of SEQ ID NO:143, an HVR-H3 comprising the amino acid sequence of SEQ ID NO:191, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:235. In some embodiments, the anti-CD137L antibody comprises an HVR-H1 comprising the amino acid sequence of SEQ ID NO:96, an HVR-H2 comprising the amino acid sequence of SEQ ID NO:144, an HVR-H3 comprising the amino acid sequence of SEQ ID NO:192, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:97, an HVR-H2 comprising the amino acid sequence of SEQ ID NO:145, an HVR-H3 comprising the amino acid sequence of SEQ ID NO:193, an HVR-L1 comprising the amino acid sequence of SEQ ID NO:221, an HVR-L2 comprising the amino acid sequence of SEQ ID NO:230, and an HVR-L3 comprising the amino acid sequence of SEQ ID NO:237. In some embodiments, the anti-CD137L antibody comprises an HVR-H1 comprising the amino acid sequence of SEQ ID NO:98, an HVR-H2 comprising the amino acid sequence of SEQ ID NO:146, an HVR-H3 comprising the amino acid sequence of SEQ ID NO:194, an HVR-LI comprising the amino acid sequence of SEQ ID NO:222, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:99, an HVR-H2 comprising the amino acid sequence of SEQ ID NO:147, an HVR-H3 comprising the amino acid sequence of SEQ ID NO:195, an HVR-L1 comprising the amino acid sequence of SEQ ID NO:223, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:100, an HVR-H2 comprising the amino acid sequence of SEQ ID NO:148, an HVR-H3 comprising the amino acid sequence of SEQ ID NO:196, an HVR-L1 comprising the amino acid sequence of SEQ ID NO:223, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:101, an HVR-H2 comprising the amino acid sequence of SEQ ID NO:149, an HVR-H3 comprising the amino acid sequence of SEQ ID NO:197, an HVR-L1 comprising the amino acid sequence of SEQ ID NO:224, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:102, an HVR-H2 comprising the amino acid sequence of SEQ ID NO:150, an HVR-H3 comprising the amino acid sequence of SEQ ID NO:198, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:240. In some embodiments, the anti-CD137L antibody comprises an HVR-H1 comprising the amino acid sequence of SEQ ID NO:103, an HVR-H2 comprising the amino acid sequence of SEQ ID NO:151, an HVR-H3 comprising the amino acid sequence of SEQ ID NO:199, an HVR-L1 comprising the amino acid sequence of SEQ ID NO:223, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:104, an HVR-H2 comprising the amino acid sequence of SEQ ID NO:152, an HVR-H3 comprising the amino acid sequence of SEQ ID NO:200, an HVR-L1 comprising the amino acid sequence of SEQ ID NO:223, an HVR-L2 comprising the amino acid sequence of SEQ ID NO:229, and an HVR-L3 comprising the amino acid sequence of SEQ ID NO:240. In some embodiments, the anti-CD137L antibody comprises an HVR-H1 comprising the amino acid sequence of SEQ ID NO:105, an HVR-H2 comprising the amino acid sequence of SEQ ID NO:153, an HVR-H3 comprising the amino acid sequence of SEQ ID NO:201, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:240. In some embodiments, the anti-CD137L antibody comprises an HVR-H1 comprising the amino acid sequence of SEQ ID NO:106, an HVR-H2 comprising the amino acid sequence of SEQ ID NO:154, an HVR-H3 comprising the amino acid sequence of SEQ ID NO:202, an HVR-L1 comprising the amino acid sequence of SEQ ID NO:225, an HVR-L2 comprising the amino acid sequence of SEQ ID NO:229, and an HVR-L3 comprising the amino acid sequence of SEQ ID NO:238. In some embodiments, the anti-CD137L antibody comprises an HVR-H1 comprising the amino acid sequence of SEQ ID NO:107, an HVR-H2 comprising the amino acid sequence of SEQ ID NO:155, an HVR-H3 comprising the amino acid sequence of SEQ ID NO:203, an HVR-L1 comprising the amino acid sequence of SEQ ID NO:226, an HVR-L2 comprising the amino acid sequence of SEQ ID NO:229, and an HVR-L3 comprising the amino acid sequence of SEQ ID NO:234. In some embodiments, the anti-CD137L antibody comprises an HVR-H1 comprising the amino acid sequence of SEQ ID NO:108, an HVR-H2 comprising the amino acid sequence of SEQ ID NO:156, an HVR-H3 comprising the amino acid sequence of SEQ ID NO:204, an HVR-L1 comprising the amino acid sequence of SEQ ID NO:221, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:109, an HVR-H2 comprising the amino acid sequence of SEQ ID NO:157, an HVR-H3 comprising the amino acid sequence of SEQ ID NO:205, an HVR-L1 comprising the amino acid sequence of SEQ ID NO:219, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:110, an HVR-H2 comprising the amino acid sequence of SEQ ID NO:158, an HVR-H3 comprising the amino acid sequence of SEQ ID NO:206, an HVR-L1 comprising the amino acid sequence of SEQ ID NO:227, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:111, an HVR-H2 comprising the amino acid sequence of SEQ ID NO:159, an HVR-H3 comprising the amino acid sequence of SEQ ID NO:207, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:112, an HVR-H2 comprising the amino acid sequence of SEQ ID NO:160, an HVR-H3 comprising the amino acid sequence of SEQ ID NO:208, an HVR-L1 comprising the amino acid sequence of SEQ ID NO:213, an HVR-L2 comprising the amino acid sequence of SEQ ID NO:231, and an HVR-L3 comprising the amino acid sequence of SEQ ID NO:235. In some embodiments, the anti-CD137L antibody comprises an HVR-H1 comprising the amino acid sequence of SEQ ID NO:113, an HVR-H2 comprising the amino acid sequence of SEQ ID NO:161, an HVR-H3 comprising the amino acid sequence of SEQ ID NO:209, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:114, an HVR-H2 comprising the amino acid sequence of SEQ ID NO:162, an HVR-H3 comprising the amino acid sequence of SEQ ID NO:210, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:240. In some embodiments, the anti-CD137L antibody comprises an HVR-H1 comprising the amino acid sequence of SEQ ID NO:115, an HVR-H2 comprising the amino acid sequence of SEQ ID NO:163, an HVR-H3 comprising the amino acid sequence of SEQ ID NO:211, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232.

In some embodiments, the antibody comprises a heavy chain variable region and a light chain variable region, wherein the heavy chain variable region comprises the amino acid sequence of SEQ ID NO:52, and/or the light chain variable region comprises the amino acid sequence of SEQ ID NO:53. In some embodiments, the antibody comprises a heavy chain variable region and a light chain variable region, wherein the heavy chain variable region comprises the amino acid sequence of SEQ ID NO:54, and/or the light chain variable region comprises the amino acid sequence of SEQ ID NO:55. In some embodiments, the antibody comprises a heavy chain variable region and a light chain variable region, wherein the heavy chain variable region comprises the amino acid sequence of SEQ ID NO:56, and/or the light chain variable region comprises the amino acid sequence of SEQ ID NO:57. In some embodiments, the antibody comprises a heavy chain variable region and a light chain variable region, wherein the heavy chain variable region comprises the amino acid sequence of SEQ ID NO:58, and/or the light chain variable region comprises the amino acid sequence of SEQ ID NO:59. In some embodiments, the antibody comprises a heavy chain variable region and a light chain variable region, wherein the heavy chain variable region comprises the amino acid sequence of SEQ ID NO:60, and/or the light chain variable region comprises the amino acid sequence of SEQ ID NO:61. In some embodiments, the antibody comprises a heavy chain variable region and a light chain variable region, wherein the heavy chain variable region comprises the amino acid sequence of SEQ ID NO:62, and/or the light chain variable region comprises the amino acid sequence of SEQ ID NO:63. In some embodiments, the antibody comprises a heavy chain variable region and a light chain variable region, wherein the heavy chain variable region comprises the amino acid sequence of SEQ ID NO:64, and/or the light chain variable region comprises the amino acid sequence of SEQ ID NO:65. In some embodiments, the antibody comprises a heavy chain variable region and a light chain variable region, wherein the heavy chain variable region comprises the amino acid sequence of SEQ ID NO:66, and/or the light chain variable region comprises the amino acid sequence of SEQ ID NO:67. In some embodiments, the anti-CD137L antibody comprises 1, 2, or 3 HVR sequences from a VH region comprising the amino acid sequence of SEQ ID NO:241 and/or 1, 2, or 3 HVR sequences from a VL region comprising the amino acid sequence of SEQ ID NO:242. In some embodiments, the anti-CD137L antibody comprises a heavy chain variable (VH) region comprising the amino acid sequence of SEQ ID NO:241 and/or a light chain variable (VL) region comprising the amino acid sequence of SEQ ID NO:242. In some embodiments, the anti-CD137L antibody comprises 1, 2, or 3 HVR sequences from a VH region comprising the amino acid sequence of SEQ ID NO:243 and/or 1, 2, or 3 HVR sequences from a VL region comprising the amino acid sequence of SEQ ID NO:244. In some embodiments, the anti-CD137L antibody comprises a heavy chain variable (VH) region comprising the amino acid sequence of SEQ ID NO:243 and/or a light chain variable (VL) region comprising the amino acid sequence of SEQ ID NO:244. In some embodiments, the anti-CD137L antibody comprises 1, 2, or 3 HVR sequences from a VH region comprising the amino acid sequence of SEQ ID NO:245 and/or 1, 2, or 3 HVR sequences from a VL region comprising the amino acid sequence of SEQ ID NO:246. In some embodiments, the anti-CD137L antibody comprises a heavy chain variable (VH) region comprising the amino acid sequence of SEQ ID NO:245 and/or a light chain variable (VL) region comprising the amino acid sequence of SEQ ID NO:246. In some embodiments, the anti-CD137L antibody comprises 1, 2, or 3 HVR sequences from a VH region comprising the amino acid sequence of SEQ ID NO:247 and/or 1, 2, or 3 HVR sequences from a VL region comprising the amino acid sequence of SEQ ID NO:248. In some embodiments, the anti-CD137L antibody comprises a heavy chain variable (VH) region comprising the amino acid sequence of SEQ ID NO:247 and/or a light chain variable (VL) region comprising the amino acid sequence of SEQ ID NO:248. In some embodiments, the anti-CD137L antibody comprises 1, 2, or 3 HVR sequences from a VH region comprising the amino acid sequence of SEQ ID NO:249 and/or 1, 2, or 3 HVR sequences from a VL region comprising the amino acid sequence of SEQ ID NO:250. In some embodiments, the anti-CD137L antibody comprises a heavy chain variable (VH) region comprising the amino acid sequence of SEQ ID NO:249 and/or a light chain variable (VL) region comprising the amino acid sequence of SEQ ID NO:250. In some embodiments, the anti-CD137L antibody comprises 1, 2, or 3 HVR sequences from a VH region comprising the amino acid sequence of SEQ ID NO:251 and/or 1, 2, or 3 HVR sequences from a VL region comprising the amino acid sequence of SEQ ID NO:252. In some embodiments, the anti-CD137L antibody comprises a heavy chain variable (VH) region comprising the amino acid sequence of SEQ ID NO:251 and/or a light chain variable (VL) region comprising the amino acid sequence of SEQ ID NO:252. In some embodiments, the anti-CD137L antibody comprises 1, 2, or 3 HVR sequences from a VH region comprising the amino acid sequence of SEQ ID NO:253 and/or 1, 2, or 3 HVR sequences from a VL region comprising the amino acid sequence of SEQ ID NO:254. In some embodiments, the anti-CD137L antibody comprises a heavy chain variable (VH) region comprising the amino acid sequence of SEQ ID NO:253 and/or a light chain variable (VL) region comprising the amino acid sequence of SEQ ID NO:254. In some embodiments, the anti-CD137L antibody comprises 1, 2, or 3 HVR sequences from a VH region comprising the amino acid sequence of SEQ ID NO:255 and/or 1, 2, or 3 HVR sequences from a VL region comprising the amino acid sequence of SEQ ID NO:256. In some embodiments, the anti-CD137L antibody comprises a heavy chain variable (VH) region comprising the amino acid sequence of SEQ ID NO:255 and/or a light chain variable (VL) region comprising the amino acid sequence of SEQ ID NO:256. In some embodiments, the anti-CD137L antibody comprises 1, 2, or 3 HVR sequences from a VH region comprising the amino acid sequence of SEQ ID NO:257 and/or 1, 2, or 3 HVR sequences from a VL region comprising the amino acid sequence of SEQ ID NO:258. In some embodiments, the anti-CD137L antibody comprises a heavy chain variable (VH) region comprising the amino acid sequence of SEQ ID NO:257 and/or a light chain variable (VL) region comprising the amino acid sequence of SEQ ID NO:258. In some embodiments, the anti-CD137L antibody comprises 1, 2, or 3 HVR sequences from a VH region comprising the amino acid sequence of SEQ ID NO:259 and/or 1, 2, or 3 HVR sequences from a VL region comprising the amino acid sequence of SEQ ID NO:260. In some embodiments, the anti-CD137L antibody comprises a heavy chain variable (VH) region comprising the amino acid sequence of SEQ ID NO:259 and/or a light chain variable (VL) region comprising the amino acid sequence of SEQ ID NO:260. In some embodiments, the anti-CD137L antibody comprises 1, 2, or 3 HVR sequences from a VH region comprising the amino acid sequence of SEQ ID NO:261 and/or 1, 2, or 3 HVR sequences from a VL region comprising the amino acid sequence of SEQ ID NO:262. In some embodiments, the anti-CD137L antibody comprises a heavy chain variable (VH) region comprising the amino acid sequence of SEQ ID NO:261 and/or a light chain variable (VL) region comprising the amino acid sequence of SEQ ID NO:262. In some embodiments, the anti-CD137L antibody comprises 1, 2, or 3 HVR sequences from a VH region comprising the amino acid sequence of SEQ ID NO:263 and/or 1, 2, or 3 HVR sequences from a VL region comprising the amino acid sequence of SEQ ID NO:264. In some embodiments, the anti-CD137L antibody comprises a heavy chain variable (VH) region comprising the amino acid sequence of SEQ ID NO:263 and/or a light chain variable (VL) region comprising the amino acid sequence of SEQ ID NO:264. In some embodiments, the anti-CD137L antibody comprises 1, 2, or 3 HVR sequences from a VH region comprising the amino acid sequence of SEQ ID NO:265 and/or 1, 2, or 3 HVR sequences from a VL region comprising the amino acid sequence of SEQ ID NO:266. In some embodiments, the anti-CD137L antibody comprises a heavy chain variable (VH) region comprising the amino acid sequence of SEQ ID NO:265 and/or a light chain variable (VL) region comprising the amino acid sequence of SEQ ID NO:266. In some embodiments, the anti-CD137L antibody comprises 1, 2, or 3 HVR sequences from a VH region comprising the amino acid sequence of SEQ ID NO:267 and/or 1, 2, or 3 HVR sequences from a VL region comprising the amino acid sequence of SEQ ID NO:268. In some embodiments, the anti-CD137L antibody comprises a heavy chain variable (VH) region comprising the amino acid sequence of SEQ ID NO:267 and/or a light chain variable (VL) region comprising the amino acid sequence of SEQ ID NO:268. In some embodiments, the anti-CD137L antibody comprises 1, 2, or 3 HVR sequences from a VH region comprising the amino acid sequence of SEQ ID NO:269 and/or 1, 2, or 3 HVR sequences from a VL region comprising the amino acid sequence of SEQ ID NO:270. In some embodiments, the anti-CD137L antibody comprises a heavy chain variable (VH) region comprising the amino acid sequence of SEQ ID NO:269 and/or a light chain variable (VL) region comprising the amino acid sequence of SEQ ID NO:270. In some embodiments, the anti-CD137L antibody comprises 1, 2, or 3 HVR sequences from a VH region comprising the amino acid sequence of SEQ ID NO:271 and/or 1, 2, or 3 HVR sequences from a VL region comprising the amino acid sequence of SEQ ID NO:272. In some embodiments, the anti-CD137L antibody comprises a heavy chain variable (VH) region comprising the amino acid sequence of SEQ ID NO:271 and/or a light chain variable (VL) region comprising the amino acid sequence of SEQ ID NO:272. In some embodiments, the anti-CD137L antibody comprises 1, 2, or 3 HVR sequences from a VH region comprising the amino acid sequence of SEQ ID NO:273 and/or 1, 2, or 3 HVR sequences from a VL region comprising the amino acid sequence of SEQ ID NO:274. In some embodiments, the anti-CD137L antibody comprises a heavy chain variable (VH) region comprising the amino acid sequence of SEQ ID NO:273 and/or a light chain variable (VL) region comprising the amino acid sequence of SEQ ID NO:274. In some embodiments, the anti-CD137L antibody comprises 1, 2, or 3 HVR sequences from a VH region comprising the amino acid sequence of SEQ ID NO:275 and/or 1, 2, or 3 HVR sequences from a VL region comprising the amino acid sequence of SEQ ID NO:276. In some embodiments, the anti-CD137L antibody comprises a heavy chain variable (VH) region comprising the amino acid sequence of SEQ ID NO:275 and/or a light chain variable (VL) region comprising the amino acid sequence of SEQ ID NO:276. In some embodiments, the anti-CD137L antibody comprises 1, 2, or 3 HVR sequences from a VH region comprising the amino acid sequence of SEQ ID NO:277 and/or 1, 2, or 3 HVR sequences from a VL region comprising the amino acid sequence of SEQ ID NO:278. In some embodiments, the anti-CD137L antibody comprises a heavy chain variable (VH) region comprising the amino acid sequence of SEQ ID NO:277 and/or a light chain variable (VL) region comprising the amino acid sequence of SEQ ID NO:278. In some embodiments, the anti-CD137L antibody comprises 1, 2, or 3 HVR sequences from a VH region comprising the amino acid sequence of SEQ ID NO:279 and/or 1, 2, or 3 HVR sequences from a VL region comprising the amino acid sequence of SEQ ID NO:280. In some embodiments, the anti-CD137L antibody comprises a heavy chain variable (VH) region comprising the amino acid sequence of SEQ ID NO:279 and/or a light chain variable (VL) region comprising the amino acid sequence of SEQ ID NO:280. In some embodiments, the anti-CD137L antibody comprises 1, 2, or 3 HVR sequences from a VH region comprising the amino acid sequence of SEQ ID NO:281 and/or 1, 2, or 3 HVR sequences from a VL region comprising the amino acid sequence of SEQ ID NO:282. In some embodiments, the anti-CD137L antibody comprises a heavy chain variable (VH) region comprising the amino acid sequence of SEQ ID NO:281 and/or a light chain variable (VL) region comprising the amino acid sequence of SEQ ID NO:282. In some embodiments, the anti-CD137L antibody comprises 1, 2, or 3 HVR sequences from a VH region comprising the amino acid sequence of SEQ ID NO:283 and/or 1, 2, or 3 HVR sequences from a VL region comprising the amino acid sequence of SEQ ID NO:284. In some embodiments, the anti-CD137L antibody comprises a heavy chain variable (VH) region comprising the amino acid sequence of SEQ ID NO:283 and/or a light chain variable (VL) region comprising the amino acid sequence of SEQ ID NO:284. In some embodiments, the anti-CD137L antibody comprises 1, 2, or 3 HVR sequences from a VH region comprising the amino acid sequence of SEQ ID NO:285 and/or 1, 2, or 3 HVR sequences from a VL region comprising the amino acid sequence of SEQ ID NO:286. In some embodiments, the anti-CD137L antibody comprises a heavy chain variable (VH) region comprising the amino acid sequence of SEQ ID NO:285 and/or a light chain variable (VL) region comprising the amino acid sequence of SEQ ID NO:286. In some embodiments, the anti-CD137L antibody comprises 1, 2, or 3 HVR sequences from a VH region comprising the amino acid sequence of SEQ ID NO:287 and/or 1, 2, or 3 HVR sequences from a VL region comprising the amino acid sequence of SEQ ID NO:288. In some embodiments, the anti-CD137L antibody comprises a heavy chain variable (VH) region comprising the amino acid sequence of SEQ ID NO:287 and/or a light chain variable (VL) region comprising the amino acid sequence of SEQ ID NO:288. In some embodiments, the anti-CD137L antibody comprises 1, 2, or 3 HVR sequences from a VH region comprising the amino acid sequence of SEQ ID NO:289 and/or 1, 2, or 3 HVR sequences from a VL region comprising the amino acid sequence of SEQ ID NO:290. In some embodiments, the anti-CD137L antibody comprises a heavy chain variable (VH) region comprising the amino acid sequence of SEQ ID NO:289 and/or a light chain variable (VL) region comprising the amino acid sequence of SEQ ID NO:290. In some embodiments, the anti-CD137L antibody comprises 1, 2, or 3 HVR sequences from a VH region comprising the amino acid sequence of SEQ ID NO:291 and/or 1, 2, or 3 HVR sequences from a VL region comprising the amino acid sequence of SEQ ID NO:292. In some embodiments, the anti-CD137L antibody comprises a heavy chain variable (VH) region comprising the amino acid sequence of SEQ ID NO:291 and/or a light chain variable (VL) region comprising the amino acid sequence of SEQ ID NO:292. In some embodiments, the anti-CD137L antibody comprises 1, 2, or 3 HVR sequences from a VH region comprising the amino acid sequence of SEQ ID NO:293 and/or 1, 2, or 3 HVR sequences from a VL region comprising the amino acid sequence of SEQ ID NO:294. In some embodiments, the anti-CD137L antibody comprises a heavy chain variable (VH) region comprising the amino acid sequence of SEQ ID NO:293 and/or a light chain variable (VL) region comprising the amino acid sequence of SEQ ID NO:294. In some embodiments, the anti-CD137L antibody comprises 1, 2, or 3 HVR sequences from a VH region comprising the amino acid sequence of SEQ ID NO:295 and/or 1, 2, or 3 HVR sequences from a VL region comprising the amino acid sequence of SEQ ID NO:296. In some embodiments, the anti-CD137L antibody comprises a heavy chain variable (VH) region comprising the amino acid sequence of SEQ ID NO:295 and/or a light chain variable (VL) region comprising the amino acid sequence of SEQ ID NO:296. In some embodiments, the anti-CD137L antibody comprises 1, 2, or 3 HVR sequences from a VH region comprising the amino acid sequence of SEQ ID NO:297 and/or 1, 2, or 3 HVR sequences from a VL region comprising the amino acid sequence of SEQ ID NO:298. In some embodiments, the anti-CD137L antibody comprises a heavy chain variable (VH) region comprising the amino acid sequence of SEQ ID NO:297 and/or a light chain variable (VL) region comprising the amino acid sequence of SEQ ID NO:298. In some embodiments, the anti-CD137L antibody comprises 1, 2, or 3 HVR sequences from a VH region comprising the amino acid sequence of SEQ ID NO:299 and/or 1, 2, or 3 HVR sequences from a VL region comprising the amino acid sequence of SEQ ID NO:300. In some embodiments, the anti-CD137L antibody comprises a heavy chain variable (VH) region comprising the amino acid sequence of SEQ ID NO:299 and/or a light chain variable (VL) region comprising the amino acid sequence of SEQ ID NO:300. In some embodiments, the anti-CD137L antibody comprises 1, 2, or 3 HVR sequences from a VH region comprising the amino acid sequence of SEQ ID NO:301 and/or 1, 2, or 3 HVR sequences from a VL region comprising the amino acid sequence of SEQ ID NO:302. In some embodiments, the anti-CD137L antibody comprises a heavy chain variable (VH) region comprising the amino acid sequence of SEQ ID NO:301 and/or a light chain variable (VL) region comprising the amino acid sequence of SEQ ID NO:302. In some embodiments, the anti-CD137L antibody comprises 1, 2, or 3 HVR sequences from a VH region comprising the amino acid sequence of SEQ ID NO:303 and/or 1, 2, or 3 HVR sequences from a VL region comprising the amino acid sequence of SEQ ID NO:304. In some embodiments, the anti-CD137L antibody comprises a heavy chain variable (VH) region comprising the amino acid sequence of SEQ ID NO:303 and/or a light chain variable (VL) region comprising the amino acid sequence of SEQ ID NO:304. In some embodiments, the anti-CD137L antibody comprises 1, 2, or 3 HVR sequences from a VH region comprising the amino acid sequence of SEQ ID NO:305 and/or 1, 2, or 3 HVR sequences from a VL region comprising the amino acid sequence of SEQ ID NO:306. In some embodiments, the anti-CD137L antibody comprises a heavy chain variable (VH) region comprising the amino acid sequence of SEQ ID NO:305 and/or a light chain variable (VL) region comprising the amino acid sequence of SEQ ID NO:306. In some embodiments, the anti-CD137L antibody comprises 1, 2, or 3 HVR sequences from a VH region comprising the amino acid sequence of SEQ ID NO:307 and/or 1, 2, or 3 HVR sequences from a VL region comprising the amino acid sequence of SEQ ID NO:308. In some embodiments, the anti-CD137L antibody comprises a heavy chain variable (VH) region comprising the amino acid sequence of SEQ ID NO:307 and/or a light chain variable (VL) region comprising the amino acid sequence of SEQ ID NO: 308. In some embodiments, the anti-CD137L antibody comprises 1, 2, or 3 HVR sequences from a VH region comprising the amino acid sequence of SEQ ID NO:309 and/or 1, 2, or 3 HVR sequences from a VL region comprising the amino acid sequence of SEQ ID NO:310. In some embodiments, the anti-CD137L antibody comprises a heavy chain variable (VH) region comprising the amino acid sequence of SEQ ID NO:309 and/or a light chain variable (VL) region comprising the amino acid sequence of SEQ ID NO:310. In some embodiments, the anti-CD137L antibody comprises 1, 2, or 3 HVR sequences from a VH region comprising the amino acid sequence of SEQ ID NO:311 and/or 1, 2, or 3 HVR sequences from a VL region comprising the amino acid sequence of SEQ ID NO:312. In some embodiments, the anti-CD137L antibody comprises a heavy chain variable (VH) region comprising the amino acid sequence of SEQ ID NO:311 and/or a light chain variable (VL) region comprising the amino acid sequence of SEQ ID NO:312. In some embodiments, the anti-CD137L antibody comprises 1, 2, or 3 HVR sequences from a VH region comprising the amino acid sequence of SEQ ID NO:313 and/or 1, 2, or 3 HVR sequences from a VL region comprising the amino acid sequence of SEQ ID NO:314. In some embodiments, the anti-CD137L antibody comprises a heavy chain variable (VH) region comprising the amino acid sequence of SEQ ID NO:313 and/or a light chain variable (VL) region comprising the amino acid sequence of SEQ ID NO:314. In some embodiments, the anti-CD137L antibody comprises 1, 2, or 3 HVR sequences from a VH region comprising the amino acid sequence of SEQ ID NO:315 and/or 1, 2, or 3 HVR sequences from a VL region comprising the amino acid sequence of SEQ ID NO:316. In some embodiments, the anti-CD137L antibody comprises a heavy chain variable (VH) region comprising the amino acid sequence of SEQ ID NO:315 and/or a light chain variable (VL) region comprising the amino acid sequence of SEQ ID NO: 316. In some embodiments, the anti-CD137L antibody comprises 1, 2, or 3 HVR sequences from a VH region comprising the amino acid sequence of SEQ ID NO:317 and/or 1, 2, or 3 HVR sequences from a VL region comprising the amino acid sequence of SEQ ID NO:318. In some embodiments, the anti-CD137L antibody comprises a heavy chain variable (VH) region comprising the amino acid sequence of SEQ ID NO:317 and/or a light chain variable (VL) region comprising the amino acid sequence of SEQ ID NO:318. In some embodiments, the anti-CD137L antibody comprises 1, 2, or 3 HVR sequences from a VH region comprising the amino acid sequence of SEQ ID NO:319 and/or 1, 2, or 3 HVR sequences from a VL region comprising the amino acid sequence of SEQ ID NO:320. In some embodiments, the anti-CD137L antibody comprises a heavy chain variable (VH) region comprising the amino acid sequence of SEQ ID NO:319 and/or a light chain variable (VL) region comprising the amino acid sequence of SEQ ID NO:320. In some embodiments, the anti-CD137L antibody comprises 1, 2, or 3 HVR sequences from a VH region comprising the amino acid sequence of SEQ ID NO:321 and/or 1, 2, or 3 HVR sequences from a VL region comprising the amino acid sequence of SEQ ID NO:322. In some embodiments, the anti-CD137L antibody comprises a heavy chain variable (VH) region comprising the amino acid sequence of SEQ ID NO:321 and/or a light chain variable (VL) region comprising the amino acid sequence of SEQ ID NO:322. In some embodiments, the anti-CD137L antibody comprises 1, 2, or 3 HVR sequences from a VH region comprising the amino acid sequence of SEQ ID NO:323 and/or 1, 2, or 3 HVR sequences from a VL region comprising the amino acid sequence of SEQ ID NO:324. In some embodiments, the anti-CD137L antibody comprises a heavy chain variable (VH) region comprising the amino acid sequence of SEQ ID NO:323 and/or a light chain variable (VL) region comprising the amino acid sequence of SEQ ID NO:324. In some embodiments, the anti-CD137L antibody comprises 1, 2, or 3 HVR sequences from a VH region comprising the amino acid sequence of SEQ ID NO:325 and/or 1, 2, or 3 HVR sequences from a VL region comprising the amino acid sequence of SEQ ID NO:326. In some embodiments, the anti-CD137L antibody comprises a heavy chain variable (VH) region comprising the amino acid sequence of SEQ ID NO:325 and/or a light chain variable (VL) region comprising the amino acid sequence of SEQ ID NO:326. In some embodiments, the anti-CD137L antibody comprises 1, 2, or 3 HVR sequences from a VH region comprising the amino acid sequence of SEQ ID NO:327 and/or 1, 2, or 3 HVR sequences from a VL region comprising the amino acid sequence of SEQ ID NO:328. In some embodiments, the anti-CD137L antibody comprises a heavy chain variable (VH) region comprising the amino acid sequence of SEQ ID NO:327 and/or a light chain variable (VL) region comprising the amino acid sequence of SEQ ID NO:328. In some embodiments, the anti-CD137L antibody comprises 1, 2, or 3 HVR sequences from a VH region comprising the amino acid sequence of SEQ ID NO:329 and/or 1, 2, or 3 HVR sequences from a VL region comprising the amino acid sequence of SEQ ID NO:330. In some embodiments, the anti-CD137L antibody comprises a heavy chain variable (VH) region comprising the amino acid sequence of SEQ ID NO:329 and/or a light chain variable (VL) region comprising the amino acid sequence of SEQ ID NO:330. In some embodiments, the anti-CD137L antibody comprises 1, 2, or 3 HVR sequences from a VH region comprising the amino acid sequence of SEQ ID NO:331 and/or 1, 2, or 3 HVR sequences from a VL region comprising the amino acid sequence of SEQ ID NO:332. In some embodiments, the anti-CD137L antibody comprises a heavy chain variable (VH) region comprising the amino acid sequence of SEQ ID NO:331 and/or a light chain variable (VL) region comprising the amino acid sequence of SEQ ID NO:332. In some embodiments, the anti-CD137L antibody comprises 1, 2, or 3 HVR sequences from a VH region comprising the amino acid sequence of SEQ ID NO:333 and/or 1, 2, or 3 HVR sequences from a VL region comprising the amino acid sequence of SEQ ID NO:334. In some embodiments, the anti-CD137L antibody comprises a heavy chain variable (VH) region comprising the amino acid sequence of SEQ ID NO:333 and/or a light chain variable (VL) region comprising the amino acid sequence of SEQ ID NO:334. In some embodiments, the anti-CD137L antibody comprises 1, 2, or 3 HVR sequences from a VH region comprising the amino acid sequence of SEQ ID NO:335 and/or 1, 2, or 3 HVR sequences from a VL region comprising the amino acid sequence of SEQ ID NO:336. In some embodiments, the anti-CD137L antibody comprises a heavy chain variable (VH) region comprising the amino acid sequence of SEQ ID NO:335 and/or a light chain variable (VL) region comprising the amino acid sequence of SEQ ID NO:336.

In some embodiments, the antibody comprises a non-human Fc region. In some embodiments, the antibody comprises a mouse Fc region. In some embodiments, the antibody comprises a mouse IgG2a Fc region.

In another aspect, provided herein are polynucleotides (e.g., isolated polynucleotides) encoding the antibody or antigen-binding fragment according to any one of the above embodiments. Also provided herein are vectors (e.g., expression vectors) comprising any of the polynucleotides according to any one of the above embodiments. Also provided herein are host cells (e.g., isolated host cells) comprising a polynucleotide or vector according to any one of the above embodiments. Also provided herein are methods of making or producing an antibody or antigen-binding fragment thereof, comprising culturing a host cell according to any one of the above embodiments under conditions suitable for producing the antibody or antigen-binding fragment. In some embodiments, the methods further comprise recovering the antibody or antigen-binding fragment produced by the cell.

In another aspect, provided herein are methods of detecting a level of expression of human CD137L in a sample. In some embodiments, the methods comprise: contacting a human tissue sample with an antibody or antigen-binding fragment according to any one of the above embodiments; and detecting binding of the antibody or antigen-binding fragment to the sample, wherein binding of the antibody or antigen-binding fragment to the sample (e.g., a level of binding of the antibody or antigen-binding fragment to the sample) indicates the level of expression of human CD137L in the sample. In some embodiments, binding of the antibody or antigen-binding fragment to the sample is detected in (c) by immunohistochemistry (IHC). In another aspect, provided herein are methods of treating or delaying progression of cancer in a subject in need thereof. In some embodiments, the methods comprise: obtaining a human tissue sample from the individual; measuring level of expression of CD137L in the sample using an antibody or antigen-binding fragment according to any one of the above embodiments; and if the level of expression of CD137L in the sample is lower than a reference level, administering an effective amount of an anti-CD137 antibody to the individual. In some embodiments according to any of the embodiments described herein, the methods further comprise obtaining a human tissue sample. In some embodiments, the methods comprise administering an effective amount of an anti-CD137 antibody to an individual, wherein level of expression of CD137L has been detected in a sample obtained from the individual using an antibody or antigen-binding fragment according to any one of the above embodiments. In some embodiments, the level of expression of CD137L in the sample is measured using IHC. In some embodiments, the level of expression of CD137L in the sample is below the limit of detection. In some embodiments, the sample is a fixed sample. In some embodiments, the sample is a formalin-fixed paraffin-embedded (FFPE) sample. In some embodiments, the sample is a tumor biopsy sample. In some embodiments, the sample is a liquid tumor sample. In some embodiments, the sample comprises one or more cancer cells. In some embodiments, the sample is a tumor sample from the cancer of the individual. In some embodiments, the level of expression of CD137L is the level of expression of CD137L by cancer cells. In some embodiments, the methods further comprise administering to the subject a therapeutically effective amount of at least one additional therapeutic agent. In some embodiments, the at least one additional therapeutic agent is selected from the group consisting of viral gene therapy, immune checkpoint inhibitor, target therapy, radiation therapy, and chemotherapy. In some embodiments, the at least one additional therapeutic agent is selected from the group consisting of pomalyst, revlimid, lenalidomide, pomalidomide, thalidomide, a DNA-alkylating platinum-containing derivative, cisplatin, 5-fluorouracil, cyclophosphamide, an anti-CTLA4 antibody, an anti-PD-1 antibody, an anti-PD-LI antibody, an anti-CD20 antibody, an anti-CD40 antibody, an anti-DR5 antibody, an anti-CD1d antibody, an anti-TIM3 antibody, an anti-SLAMF7 antibody, an anti-KIR receptor antibody, an anti-OX40 antibody, an anti-HER2 antibody, an anti-ErbB-2 antibody, an anti-EGFR antibody, cetuximab, rituximab, trastuzumab, pembrolizumab, radiotherapy, single dose radiation, fractionated radiation, focal radiation, whole organ radiation, IL-12, IFNα, GM-CSF, a chimeric antigen receptor, adoptively transferred T cells, an anti-cancer vaccine, and an oncolytic virus.

It is to be understood that one, some, or all of the properties of the various embodiments described above and herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIG. 1 shows high specificity of CD137L binding by purified anti-CD137L antibodies (as indicated), as measured by ELISA assays using CD137L peptides 01 and 02, as well as four other peptides as negative controls.

FIGS. 2A-2D show a comparison of binding specificity between TY23561 and the commercial Reference-1 antibody. Binding to a CHO-S cell line stably overexpressing human CD137L (CHO-S-CD137L) or to parental CHO-S cells was assayed by flow cytometry. The positive control cell CHO-S-CD137L and the negative control CHO-S cells were washed, fixed and permabilized in a similar fashion as in immunohistochemistry, followed by analysis through Beckman CytoFlex. (FIG. 2A) Anti-CD137L antibody TY23561 (0.4 nM or 2 nM) was used to stain CHO-S-CD137L or CHO-S cell lines. (FIG. 2B) The commercial anti-CD137L antibody Reference-1 (0.4 nM or 2 nM) was used to stain CHO-S-CD137L or CHO-S cell lines. (FIG. 2C) A negative isotype control antibody (0.4 nM or 2 nM) was used to stain CHO-S-CD137L or CHO-S cell lines. (FIG. 2D) Specificity index (defined as MFI(CHO-S-CD137L)/MFI(CHO-S)) was calculated for TY23557, TY23561, the Reference-1 and isotype control antibodies. TY23561 shows much higher specificity index than the commercial Reference-1.

FIGS. 3A-3C show immunofluorescence staining of TY23557, TY23561, the Reference-1 and isotype control antibodies at the indicated concentrations. The positive control CHO-S-CD137L cell line and the negative control CHO-S cells were washed, fixed and permabilized in a similar fashion as in immunohistochemistry (IHC), followed by analysis through fluorescence microscopy. (FIG. 3A) CHO-S-CD137L cells were stained with TY23557 or TY23561 (0.4 nM or 40 nM). (FIG. 3B) CHO-S-CD137L cells were stained with Reference-1 (6.66 nM) or negative isotype control (40 nM). (FIG. 3C) CHO-S cells were stained with TY23557 (40 nM), TY23561 (40 nM), negative isotype control (40 nM) or Reference-1 (6.66 nM).

FIG. 4 shows the specificity of immunohistochemistry (IHC) staining of anti-CD137 ligand antibodies in cultured cells. The positive control cell CHO-S-CD137L and the negative control CHO-S cells were FFPE processed and sectioned. Antibody staining with different anti-CD137 antibodies was then performed following optimized IHC conditions, and CD137L signals were detected with an HRP-labeled anti-mouse IgG followed by DAB chromogenic reaction. CD137L proteins were stained Brown, and cell nuclei were stained Blue by Hematoxylin.

FIG. 5 shows the specificity of immunohistochemistry (IHC) staining of anti-CD137L antibodies in human Tonsil tissue sections. Human Tonsil FFPE sections were stained with anti-CD137L antibodies, followed by an HRP-labeled 2^{nd} anti-mouse IgG detection and DAB chromogenic reaction. CD137L proteins were stained Brown, and cell nuclei were stained Blue by Hematoxylin.

FIGS. 6A & 6B the immunohistochemistry (IHC) staining of CD137 ligand in patient samples by the anti-CD137L antibody TY23561. FIG. 6A shows the immunohistochemistry (IHC) staining of CD137 ligand in lung cancer patient samples using TY23561. FIG. 6B shows the immunohistochemistry (IHC) staining of CD137 ligand in lymphoma patient samples using TY23561. FFPE tumor sections from different patients with lung cancers or lymphoma were stained with TY23561 and an HRP-labeled 2^{nd} anti-mouse IgG, followed by DAB chromogenic reaction. An automated Leica Bond-RX immunostainer was used at ER1 retrieval setting, and staining was optimized using appropriate FFPE controls. The stained sections were scanned with a 3D HISTECH Pannoramic MIDI. CD137L proteins were stained Brown, and cell nuclei were stained Blue by Hematoxylin.

### DETAILED DESCRIPTION

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

Before describing the present disclosure in detail, it is to be understood that this present disclosure is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure described herein include “comprising,” “consisting,” and “consisting essentially of” aspects and embodiments.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “antibody” is used herein in the broadest sense and specifically covers monoclonal antibodies (including full length monoclonal antibodies), polyclonal antibodies, multispecific antibodies (e.g., bispecific antibodies), and antibody fragments (e.g., a single-chain variable fragment or scFv) so long as they exhibit the desired biological activity.

The term “antibody” is an art-recognized term and may refer to an antigen-binding protein (i.e, immunoglobulin) having a basic four-polypeptide chain structure consisting of two identical heavy (H) chains and two identical light (L) chains. Each L chain is linked to an H chain by one covalent disulfide bond, while the two H chains are linked to each other by one or more disulfide bonds depending on the H chain isotype. Each heavy chain has, at the N-terminus, a variable region (abbreviated herein as V_{H}) followed by a constant region. The heavy chain constant region is comprised of three domains, C_{H1}, C_{H2} and C_{H3}. Each light chain has, at the N-terminus, a variable region (abbreviated herein as VL) followed by a constant region at its other end. The light chain constant region is comprised of one domain, C_{L}. The V_{L} is aligned with the V_{H} and the C_{L} is aligned with the first constant domain of the heavy chain (CH1). The pairing of a V_{H} and V_{L} together forms a single antigen-binding site. An IgM antibody consists of 5 of the basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain.

The V_{H} and V_{L} regions can be further subdivided into regions of hypervariability, termed hyper-variable regions (HVR) based on the structural and sequence analysis. HVRs are interspersed with regions that are more conserved, termed framework regions (FW). For comparison, the Kabat CDR definition by Yvonne Chen, et al. (Selection and Analysis of an Optimized Anti-VEGF Antibody: Crystal Structure of an Affinity-matured Fab in Complex with Antigen, J. Mol. Biol. (1999) 293, 865-881) is listed below. Each V_{H} and V_{L} is composed of three HVRs and four FWs, arranged from amino-terminus to carboxy-terminus in the following order: FW1, HVR1, FW2, HVR2, FW3, HVR3, FW4. Throughout the present disclosure, the three HVRs of the heavy chain are referred to as HVR_H1, HVR_H2, and HVR_H3. Similarly, the three HVRs of the light chain are referred to as HVR_L 1, HVR_L2, and HVR_L3.

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. Within light and heavy chains, the variable and constant regions are joined by a “J” region of about 12 or more amino acids, with the heavy chain also including a “D” region of about 10 or more amino acids. See generally, Fundamental Immunology Ch. 7 (Paul, W., ed., 2^{nd} ed. Raven Press, N.Y. (1989)).

The L chain from any vertebrate species can be assigned to one of two clearly distinct types, called kappa and lambda, based on the amino acid sequences of their constant domains. Depending on the amino acid sequence of the constant domain of their heavy chains (CH), antibodies can be assigned to different classes or isotypes. There are five classes of antibodies: IgA, IgD, IgE, IgG, and IgM, having heavy chains designated α (alpha), δ (delta), ε (epsilon), γ (gamma), and μ (mu), respectively. The IgG class of antibody can be further classified into four subclasses IgG1, IgG2, IgG3, and IgG4 by the gamma heavy chains, Y1-Y4, respectively.

The term “antibody derivative” or “derivative” of an antibody refers to a molecule that is capable of binding to the same antigen (e.g., CD137L) that the antibody binds to and comprises an amino acid sequence of the antibody linked to an additional molecular entity. The amino acid sequence of the antibody that is contained in the antibody derivative may be a full-length heavy chain, a full-length light chain, any portion or portions of a full-length heavy chain, any portion or portions of the full-length light chain of the antibody, any other fragment(s) of an antibody, or the complete antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional molecular entity by chemical coupling, genetic fusion, noncovalent association, or otherwise. The term “antibody derivative” also encompasses chimeric antibodies, humanized antibodies, and molecules that are derived from modifications of the amino acid sequences of an antibody (e.g., an anti-CD137L antibody), such as conservation amino acid substitutions, additions, and insertions.

The term “antigen-binding fragment” or “antigen binding portion” of an antibody refers to one or more portions of an antibody that retain the ability to bind to the antigen that the antibody bonds to (e.g., CD137L). Examples of “antigen-binding fragment” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., Nature 341:544-546 (1989)), which consists of a V_{H} domain; and (vi) an isolated complementarity determining region (CDR).

The term “binding molecule” encompasses (1) antibody, (2) antigen-binding fragment of an antibody, and (3) derivative of an antibody, each as defined herein.

The term “binding CD137L,” “binds CD137L,” “binding to CD137L,” or “binds to CD137L” refers to the binding of a binding molecule, as defined herein, to the human CD137L in an in vitro assay, such as a Biacore assay, with an affinity (K_{D}) of 100 nM or less.

The terms “CD137L” and “CD137 ligand” are used interchangeably in the present application, and include the human CDI 37 ligand, as well as variants, isoforms, and species homologs thereof. Accordingly, a binding molecule, as defined and disclosed herein, may also bind CD137L from species other than human. In other cases, a binding molecule may be completely specific for the human CD137L and may not exhibit species or other types of cross-reactivity.

The term “anti-CD137L antibody” refers to an antibody, as defined herein, capable of binding to human CD137 ligand (CD137L).

The term “chimeric antibody” refers to an antibody that comprises amino acid sequences derived from different animal species, such as those having a variable region derived from a human antibody and a murine immunoglobulin constant region.

The term “compete for binding” refers to the interaction of two antibodies in their binding to a binding target. A first antibody competes for binding with a second antibody if binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not, be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s).

The term “epitope” refers to a part of an antigen to which an antibody (or antigen-binding fragment thereof) binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope can include various numbers of amino acids in a unique spatial conformation. Methods of determining spatial conformation of epitopes include, for example, x-ray crystallography, 2-dimensional nuclear magnetic resonance, deuterium and hydrogen exchange in combination with mass spectrometry, or site-directed mutagenesis, or all methods used in combination with computational modeling of antigen and its complex structure with its binding antibody and its variants. See, e.g., Epitope Mapping Protocols in Methods in Molecular Biology, Vol. 66, G. E. Morris, Ed. (1996). Once a desired epitope of an antigen is determined, antibodies to that epitope can be generated, e.g., using the techniques described herein. The generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

The term “germline” refers to the nucleotide sequences of the antibody genes and gene segments as they are passed from parents to offspring via the germ cells. The germline sequence is distinguished from the nucleotide sequences encoding antibodies in mature B cells which have been altered by recombination and hypermutation events during the course of B cell maturation.

The term “glycosylation sites” refers to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. The specific site of attachment is typically signaled by a sequence of amino acids, referred to herein as a “glycosylation site sequence”. The glycosylation site sequence for N-linked glycosylation is: -Asn-X-Ser- or -Asn-X-Thr-, where X may be any of the conventional amino acids, other than proline. The terms “N-linked” and “O-linked” refer to the chemical group that serves as the attachment site between the sugar molecule and the amino acid residue. N-linked sugars are attached through an amino group; O-linked sugars are attached through a hydroxyl group. The term “glycan occupancy” refers to the existence of a carbohydrate moiety linked to a glycosylation site (i.e., the glycan site is occupied). Where there are at least two potential glycosylation sites on a polypeptide, either none (0-glycan site occupancy), one (1-glycan site occupancy) or both (2-glycan site occupancy) sites can be occupied by a carbohydrate moiety.

The term “host cell” refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

The term “human antibody” refers to an antibody in which the entire amino acid sequences of the light chains and heavy chains are from the human immunoglobulin genes. A human antibody may contain murine carbohydrate chains if produced in a mouse, in a mouse cell or in a hybridoma derived from a mouse cell. Human antibodies may be prepared in a variety of ways known in the art.

The term “humanized antibody” refers to a chimeric antibody that contains amino acid residues derived from human antibody sequences. A humanized antibody may contain some or all of the CDRs or HVRs from a non-human animal or synthetic antibody while the framework and constant regions of the antibody contain amino acid residues derived from human antibody sequences.

The term “illustrative antibody” refers to any one of the antibodies described in the disclosure. These antibodies may be in any class (e.g., IgA, IgD, IgE, IgG, and IgM). Thus, each antibody identified above encompasses antibodies in all five classes that have the same amino acid sequences for the V_{L} and V_{H} regions. Further, the antibodies in the IgG class may be in any subclass (e.g., IgG1 IgG2, IgG3, and IgG4). Thus, each antibody identified above in the IgG subclass encompasses antibodies in all four subclasses that have the same amino acid sequences for the V_{L} and V_{H} regions. The amino acid sequences of the heavy chain constant regions of human antibodies in the five classes, as well as in the four IgG subclasses, are known in the art.

The term “isolated antibody” or “isolated binding molecule” refers to an antibody or a binding molecule, as defined herein, that: (1) is not associated with naturally associated components that accompany it in its native state; (2) is free of other proteins from the same species; (3) is expressed by a cell from a different species; or (4) does not occur in nature. Examples of isolated antibodies include a CD137 antibody that has been affinity purified using CD137, a CD137 antibody that has been generated by hybridomas or other cell line in vitro, and a CD137 antibody derived from a transgenic animal.

The term “kₐ” refers to the association rate constant of a particular antibody-antigen interaction, whereas the term “k_{d}” refers to the dissociation rate constant of a particular antibody-antigen interaction.

The term “K_{D}” refers to the equilibrium dissociation constant of a particular antibody-antigen interaction. It is obtained from the ratio of k_{d} to kₐ (i.e., kₐ/kₐ) and is expressed as a molar concentration (M). K_{D} is used as a measure for the affinity of an antibody's binding to its binding partner. The smaller the K_{D}, the more tightly bound the antibody is, or the higher the affinity between antibody and the antigen. For example, an antibody with a nanomolar (nM) dissociation constant binds more tightly to a particular antigen than an antibody with a micromolar (μM) dissociation constant. K_{D} values for antibodies can be determined using methods well established in the art. One method for determining the K_{D} of an antibody is by using surface plasmon resonance, typically using a biosensor system such as a Biacore® system.

The term “prevent” or “preventing,” with reference to a certain disease condition in a mammal, refers to preventing or delaying the onset of the disease, or preventing the manifestation of clinical or subclinical symptoms thereof.

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (see, e.g. Pearson, Methods Enzymol. 183:63-98 (1990), Pearson, Methods Mol. Biol. 132.185-219 (2000): Altschul et al., J. Mol. Biol. 215:403-410 (1990); Altschul et al., Nucleic Acids Res. 25.3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

The term “specifically binds” or “specifically binds to,” in reference to the interaction of a binding molecule, as defined herein, (e.g., an antibody) with its binding partner (e.g., an antigen), refers to the ability of the binding molecule to discriminate between an antigen of interest from an animal species and the antigen orthologue from a different animal species under a given set of conditions. A CD137L binding molecule is said to specifically bind to human CD137L if it binds to human CD137L at an EC50 that is below 50 percent of the EC50 at which it binds CD137L of rat or mouse as determined in an in vitro assay. Binding specificity of an antibody can be determined using methods known in the art. Examples of such methods include FACS using PHA stimulated primary cells, Western blots, ELISA-, RIA-, ECL-, IRMA-tests and peptide scans.

The term “treat”, “treating”, or “treatment”, with reference to a certain disease condition in a mammal, refers causing a desirable or beneficial effect in the mammal having the disease condition. The desirable or beneficial effect may include reduced frequency or severity of one or more symptoms of the disease (i.e., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

The term “vector” refers to a nucleic acid molecule capable of transporting a foreign nucleic acid molecule. The foreign nucleic acid molecule is linked to the vector nucleic acid molecule by a recombinant technique, such as ligation or recombination. This allows the foreign nucleic acid molecule to be multiplied, selected, further manipulated or expressed in a host cell or organism. A vector can be a plasmid, phage, transposon, cosmid, chromosome, virus, or virion. One type of vectors can be integrated into the genome of a host cell upon introduction into the host cell, and thereby are replicated along with the host genome (e.g., non-episomal mammalian vectors). Another type of vector is capable of autonomous replication in a host cell into which it is introduced (e.g., bacterial vectors having a bacterial origin of replication and episomal mammalian vectors). Another specific type of vector capable of directing the expression of expressible foreign nucleic acids to which they are operatively linked is commonly referred to as “expression vectors.” Expression vectors generally have control sequences that drive expression of the expressible foreign nucleic acids. Simpler vectors, known as “transcription vectors,” are only capable of being transcribed but not translated: they can be replicated in a target cell but not expressed. The term “vector” encompasses all types of vectors regardless of their function. Vectors capable of directing the expression of expressible nucleic acids to which they are operatively linked are commonly referred to “expression vectors.”

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. The term “amino acid analogs” refers to compounds that have the same basic chemical structure as a naturally occurring amino acid but the C-terminal carboxy group, the N-terminal amino group, or side chain functional group has been chemically modified to another functional group. The term “amino acid mimetics” refers to chemical compounds that have a structure that is different from the general chemical structure of an amino acid, but that functions similarly to a naturally occurring amino acid.

As used herein, the twenty conventional amino acids and their abbreviations follow conventional usage. See Immunology—A Synthesis (2nd Edition, E. S. Golub and D. R Gren, Eds., Sinauer Associates, Sunderland, Mass. (1991)).

The terms “polypeptide,” “protein,” and “peptide” are used interchangeably herein and may refer to polymers of two or more amino acids.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “isolated nucleic acid” refers to a nucleic acid molecule of genomic, cDNA, or synthetic origin, or a combination thereof, which is separated from other nucleic acid molecules present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid of interest.

As used herein, the term “biomarker” or “marker” refers generally to a molecule (e.g., pre-mRNA, mRNA, protein, etc.), the expression of which in or on a subject's tissue or cell, or secreted by the subject's tissue or cell, can be detected by known methods (or methods disclosed herein) and is predictive or can be used to predict (or aid prediction) for a subject's sensitivity to, and in some embodiments, to predict (or aid prediction) a subject's responsiveness to, treatment regimens (e.g., treatments comprising anti-CD137 antibodies, treatments comprising checkpoint blockade immunotherapy, etc.).

As used herein, the term “sample”, refers to a composition that is obtained or derived from a subject of interest that contains a cellular and/or other molecular entity that is to be characterized and/or identified, for example based on physical, biochemical, chemical and/or physiological characteristics.

As used herein, the term “tissue or cell sample” refers to a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells. Optionally, the tissue or cell sample is obtained from a disease tissue or organ. The tissue sample may contain compounds which are not naturally intermixed with the tissue in nature such as preservatives, anticoagulants, buffers, fixatives, nutrients, antibiotics, or the like.

As used herein, a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

The term “mammal” refers to any animal species of the Mammalia class. Examples of mammals include: humans; laboratory animals such as rats, mice, simians and guinea pigs; domestic animals such as cats, dogs, rabbits, cattle, sheep, goats, horses, and pigs; and captive wild animals such as lions, tigers, elephants, and the like.

As used herein, a “reference value” or “reference level” may be an absolute value; a relative value: a value that has an upper and/or lower limit; a range of values; an average value; a median value; a mean value; or a value as compared to a particular level or baseline level.

An “effective amount” refers to at least an amount effective, at dosages and for periods of time necessary, to achieve a one or more desired or indicated effect, including a therapeutic or prophylactic result. An effective amount can be provided in one or more administrations. For purposes of the present disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

### III. Antibodies and Methods

Overview

Certain aspects of the present disclosure relate to antibodies, or antigen-binding fragments thereof, that bind to an intracellular and/or transmembrane region of human CD137 ligand (CD137L). For example, in some embodiments, the antibodies and antigen-binding fragments of the present disclosure bind to a peptide comprising the amino acid sequence of MEYASDASLDPEAPWPPAPRARACRVLP (SEQ ID NO:1), and/or bind to a peptide comprising the amino acid sequence of MEYASDASLDPEAPWPPAPRARA (SEQ ID NO:2). Advantageously, the present disclosure provides anti-CD137L antibodies that bind to the intracellular and/or transmembrane region of human CD137L with high specificity, and demonstrates that such antibodies allow for highly robust and sensitive detection of CD137L in human tissue samples, e.g., using IHC.

Other aspects of the present disclosure relate to methods of detecting expression level of CD137L in a sample, e.g., a human tissue sample, using an antibody or antigen-binding fragment thereof disclosed herein. Such methods may find use, inter alia, in detecting CD137L expression level in a tissue sample in order to identify patients that may respond to a particular cancer therapeutic, such as treatment with an anti-CD137 antibody.

Anti-CD137L Antibodies

In some embodiments, the present disclosure relates to an anti-CD137L antibody. In some embodiments, the anti-CD137L antibody binds to human CD137L. Human CD137L (also known as TNFSF9, TNLG5A, and 4-1BB-L) is a transmembrane cytokine of the tumor necrosis factor (TNF) ligand family that acts as a ligand for CD137/TNFRSF9/4-1BB. In some embodiments, human CD137L refers to a polypeptide produced by the human CD137L gene, e.g., as represented by NCBI Gene ID NO. 8744. An exemplary CD137L polypeptide sequence is provided below (see, e.g., NCBI Ref. Seq. No. NP_003802.1).

(SEQ ID NO: 3)
MEYASDASLD PEAPWPPAPR ARACRVLPWA
LVAGLLLLLL LAAACAVFLA CPWAVSGARA
SPGSAASPRL REGPELSPDD PAGLLDLRQG
MFAQLVAQNV LLIDGPLSWY SDPGLAGVSL
TGGLSYKEDT KELVVAKAGV YYVFFQLELR
RVVAGEGSGS VSLALHLQPL RSAAGAAALA
LTVDLPPASS EARNSAFGFQ GRLLHLSAGQ
RLGVHLHTEA RARHAWQLTQ GATVLGLFRV
TPEIPAGLPS PRSE

In some embodiments, the present disclosure provides an isolated antibody that binds to human CD137L, e.g., at the intracellular and/or transmembrane domain(s). In some embodiments, the antibody does not bind to an extracellular domain or portion thereof of human CD137L. The sequence and structure of the CD137L extracellular domain are known in the art (see, e.g., Won, E. H. et al. (2010) J. Biol. Chen. 285(12):9202-9210).

In some embodiments, an antibody or antigen-binding fragment of the present disclosure binds to human CD137L at an epitope comprising the amino acid sequence of MEYASDASLDPEAPWPPAPRARACRVLP (SEQ ID NO:1) and/or comprising the amino acid sequence of MEYASDASLDPEAPWPPAPRARA (SEQ ID NO:2). In some embodiments, an antibody or antigen-binding fragment of the present disclosure binds to a peptide comprising the amino acid sequence of MEYASDASLDPEAPWPPAPRARACRVLP (SEQ ID NO:1) and/or a peptide comprising the amino acid sequence of MEYASDASLDPEAPWPPAPRARA (SEQ ID NO:2). In some embodiments, the peptide is less than 100, less than 90, less than 80, less than 70, less than 60, less than 50, less than 40, or less than 30 amino acids in length. In some embodiments, the peptide does not comprise the extracellular domain or a portion thereof of CD137L. In some embodiments, the antibody or antigen-binding fragment binds to the extracellular domain of human CD137L. In some embodiments, the antibody or antigen-binding fragment binds to an intracellular domain or non-transmembrane region of human CD137L.

In some embodiments, the antibody or antigen-binding fragment binds to a cell, e.g., a Chinese hamster ovary (CHO) cell, that expresses human CD137L. In some embodiments, the cell is fixed and/or permeabilized, e.g., to allow access of the antibody to the intracellular or transmembrane domain(s) of CD137L as expressed by the cell.

In some embodiments, the antibody or antigen-binding fragment binds to human CD137L in a fixed sample, e.g., comprising one or more fixed human cells. In some embodiments, the sample is a fixed and/or permeabilized human tissue sample, e.g., from human tonsil tissue or from human tumor tissue (e.g., a solid or liquid tumor). In some embodiments, the sample is a formalin-fixed paraffin-embedded (FFPE) sample.

In some embodiments, the anti-CD137L antibody comprises a heavy chain variable region and a light chain variable region, wherein the heavy chain variable region comprises an HVR-H1 comprising an amino acid sequence according to Formula (I): LX₁TX₂GVGVX₃WI, wherein X₁ is S, N, or T; wherein X₂ is S, A, G, or T; and wherein X₃ is S, T, A, or G (SEQ ID NO:346); an HVR-H2 comprising an amino acid sequence according to Formula (II): X₁X₂X₃IDX₄X₅X₆X₇X₈YYX₉PSX₁₀KSX₁₁L, wherein X₁ is L or I; wherein X₂ is A or G; wherein X₃ is L, V, or I; wherein X₄ is W, H, or Y; wherein X₅ is A or S; wherein X₆ is G or D; wherein X₇ is D, Y, A, or S; wherein X₈ is K or T; wherein X₉ is S or N; wherein X₁₀ is L or P; and wherein X₁₁ is R or H (SEQ TD NO:347); and an HVR-H3 comprising an amino acid sequence according to Formula (III): ARYGX₁X₂X₃YAX₄DY, wherein X₁ is Y, V, W, L, or G: wherein X₂ is S, G, R, D, or N; wherein X₃ is S, G, or N; and wherein X₄ is I, L, or M (SEQ ID NO:348); and/or wherein the light chain variable region comprises an HVR-L1 comprising an amino acid sequence according to Formula (IV): RX₁SQX₂X₃X₄X₅X₆X₇LA, wherein X₁ is A or T; wherein X₂ is S, T, or G; wherein X₃ is V or I; wherein X₄ is R, S, or H; wherein X₅ is G or N; wherein X₆ is S, N, or T; and wherein X₇ is Y or F (SEQ ID NO:349); an HVR-L2 comprising an amino acid sequence according to Formula (V): X₁AX₂X₃LX₄SGV, wherein X₁ is A or D; wherein X₂ is S or F; wherein X₃ is T or S; and wherein X₄ is Q or E (SEQ ID NO:350); and an HVR-L3 comprising an amino acid sequence according to Formula (VI): YCQQYX₁SX₂WT, wherein X₁ is S, G, or N and wherein X₂ is L, Y, S, W or F (SEQ ID NO:351).

In one aspect, the present disclosure provides an isolated antibody comprising a heavy chain variable region and a light chain variable region, wherein: (a) the heavy chain variable region comprises an HVR-H1 comprising the amino acid sequence of SEQ ID NO:4, an HVR-H2 comprising the amino acid sequence of SEQ ID NO:5, and an HVR-H3 comprising the amino acid sequence of SEQ ID NO:6, and/or the light chain variable region comprises an HVR-L1 comprising the amino acid sequence of SEQ ID NO:7, an HVR-L2 comprising the amino acid sequence of SEQ ID NO:8, and an HVR-L3 comprising the amino acid sequence of SEQ ID NO:9; (b) the heavy chain variable region comprises an HVR-H1 comprising the amino acid sequence of SEQ ID NO:10, an HVR-H2 comprising the amino acid sequence of SEQ ID NO:11, and an HVR-H3 comprising the amino acid sequence of SEQ ID NO:12, and/or the light chain variable region comprises an HVR-L1 comprising the amino acid sequence of SEQ ID NO:13, an HVR-L2 comprising the amino acid sequence of SEQ ID NO:14, and an HVR-L3 comprising the amino acid sequence of SEQ ID NO:15; (c) the heavy chain variable region comprises an HVR-H1 comprising the amino acid sequence of SEQ ID NO:16, an HVR-H2 comprising the amino acid sequence of SEQ ID NO:17, and an HVR-H3 comprising the amino acid sequence of SEQ ID NO:18, and/or the light chain variable region comprises an HVR-L1 comprising the amino acid sequence of SEQ ID NO:19, an HVR-L2 comprising the amino acid sequence of SEQ ID NO:20, and an HVR-L3 comprising the amino acid sequence of SEQ ID NO:21; (d) the heavy chain variable region comprises an HVR-H1 comprising the amino acid sequence of SEQ ID NO:22, an HVR-H2 comprising the amino acid sequence of SEQ ID NO:23, and an HVR-H3 comprising the amino acid sequence of SEQ ID NO:24, and/or the light chain variable region comprises an HVR-L1 comprising the amino acid sequence of SEQ ID NO:25, an HVR-L2 comprising the amino acid sequence of SEQ ID NO:26, and an HVR-L3 comprising the amino acid sequence of SEQ ID NO:27; (e) the heavy chain variable region comprises an HVR-H1 comprising the amino acid sequence of SEQ ID NO:28, an HVR-H2 comprising the amino acid sequence of SEQ ID NO:29, and an HVR-H3 comprising the amino acid sequence of SEQ ID NO:30, and/or the light chain variable region comprises an HVR-L1 comprising the amino acid sequence of SEQ ID NO:31, an HVR-L2 comprising the amino acid sequence of SEQ ID NO:32, and an HVR-L3 comprising the amino acid sequence of SEQ ID NO:33; (f) the heavy chain variable region comprises an HVR-H1 comprising the amino acid sequence of SEQ ID NO:34, an HVR-H2 comprising the amino acid sequence of SEQ ID NO:35, and an HVR-H3 comprising the amino acid sequence of SEQ ID NO:36, and/or the light chain variable region comprises an HVR-LI comprising the amino acid sequence of SEQ ID NO:37, an HVR-L2 comprising the amino acid sequence of SEQ ID NO:38, and an HVR-L3 comprising the amino acid sequence of SEQ ID NO:39; (g) the heavy chain variable region comprises an HVR-H1 comprising the amino acid sequence of SEQ ID NO:40, an HVR-H2 comprising the amino acid sequence of SEQ ID NO:41, and an HVR-H3 comprising the amino acid sequence of SEQ ID NO:42, and/or the light chain variable region comprises an HVR-L1 comprising the amino acid sequence of SEQ ID NO:43, an HVR-L2 comprising the amino acid sequence of SEQ ID NO:44, and an HVR-L3 comprising the amino acid sequence of SEQ ID NO:45; or (h) the heavy chain variable region comprises an HVR-H1 comprising the amino acid sequence of SEQ ID NO:46, an HVR-H2 comprising the amino acid sequence of SEQ ID NO:47, and an HVR-H3 comprising the amino acid sequence of SEQ ID NO:48, and/or the light chain variable region comprises an HVR-L1 comprising the amino acid sequence of SEQ ID NO:49, an HVR-L2 comprising the amino acid sequence of SEQ ID NO:50, and an HVR-L3 comprising the amino acid sequence of SEQ ID NO:51.

Examples of specific antibodies (or antigen binding fragments thereof) provided by the present disclosure include those listed in Tables 1-4. While the antibodies/antigen-binding fragments listed in Tables 1 and 3 are described as antibodies, and those listed in Tables 2 and 4 are described as Fab fragments, these descriptors are not intended to be limiting in any way. Thus it is contemplated herein that the antigen-binding domains of Tables 1 and 3 could also be used in an antigen-binding fragment, and those in Tables 2 and 4 could also be used in an antibody.

TABLE 1
HVR sequences of anti-CD137L antibodies

| Antibody | | | | | | |
|---|---|---|---|---|---|---|
| Name | HVR-H1 | HVR-H2 | HVR-H3 | HVR-L1 | HVR-L2 | HVR-L3 |
| TY23554 | YSIT | IGII | ARDG | RASQ | DASN | YCQQ |
| | SGYY | NPNR | WALS | DISS | LETG | YD |
| | WAWI | GSTK | GGYY | DLA | V | AWPS |
| | (SEQ | YAQK | YYYG | (SEQ | (SEQ ID | T |
| | ID NO: | FQGR | LDV | ID | NO: 4) | (SEQ ID |
| | 4) | V | (SEQ ID | NO: 7) | | NO: 9) |
| | | (SEQ ID | NO: 6) | | | |
| | | NO: 5) | | | | |
| TY23555 | YSIS | VSVI | ARYR | RASQ | AASS | YCQQ |
| | SGHY | SGSG | SGAF | SISR | LQSG | AY |
| | WAWI | GSTY | DY | WLA | V | SIWT |
| | (SEQ ID | YADS | (SEQ ID | (SEQ ID | (SEQ ID | |
| | NO: 10) | VKGR | NO: 12) | NO: 13) | NO: 14) | (SEQ ID |
| | | F | | | | NO: 15) |
| | | (SEQ ID | | | | |
| | | NO: 11) | | | | |
| TY23556 | YSIT | IGEI | TRGR | SASS | DASN | YCQQ |
| | SG | YHSG | YGLD | SVGY | RA | YG |
| | HYWG | STY | Y | VQ | TGI | SAPI |
| | WI | YSPS | (SEQ ID | (SEQ ID | (SEQ | T |
| | (SEQ | LKSR | NO: 18) | NO: 19) | ID NO: | (SEQ ID |
| | ID NO: | V | | | 20) | NO: 21) |
| | 16) | (SEQ ID | | | | |
| | | NO: 17) | | | | |
| TY23557 | FTFT | VSAI | ARGA | RASE | DASS | YCFQ |
| | GYGI | SGSG | YYGG | SITS | LES | GS |
| | HWV | GS | S | S | GV | HYPP |
| | (SEQ ID | TYYA | YYFD | YLA | (SEQ | T |
| | NO: 22) | DSVK | Y | (SEQ | ID NO: | (SEQ ID |
| | | GRF | (SEQ | ID NO: | 26) | NO: 27) |
| | | (SEQ ID | ID NO: | 25) | | |
| | | NO: 23) | 24) | | | |
| TY23558 | YSIS | IGEI | AREG | RARS | DASN | YCQQ |
| | SGHY | YHSG | SDSY | TVSF | LETG | SL |
| | WSWI | STY | GG | PYLH | V | EDPF |
| | (SEQ | YSPS | PDPF | (SEQ | (SEQ ID | T |
| | ID NO: | LKSR | DI | ID NO: | NO: 32) | (SEQ ID |
| | 28) | V | (SEQ | 31) | | NO: 33) |
| | | (SEQ ID | ID NO: | | | |
| | | NO: 29) | 30) | | | |
| TY23559 | YSIS | IGEI | TRGR | SASS | DASN | YCQQ |
| | SG | YHSG | YGLD | SVSY | RA | YS |
| | HYWA | STY | Y | | TGI | TNPL |
| | WI | YSPS | (SEQ ID | VQ | (SEQ | T |
| | (SEQ ID | LKSR | NO: 36) | (SEQ ID | ID NO: 38) | (SEQ ID |
| | NO: 34) | V | | NO: 37) | | NO: 39) |
| | | (SEQ ID | | | | |
| | | NO: 35) | | | | |
| TY23561 | FSLS | LALI | ARYG | RASQ | AAST | YCQQ |
| | TS | DWAG | YSSY | SVRG | LQSG | YS |
| | GVGV | D | AL | SYLA | V | SLWT |
| | SWI | KYYS | DY | (SEQ | (SEQ ID | (SEQ ID |
| | (SEQ | PSLK | (SEQ ID | ID NO: | NO: 44) | NO: 45) |
| | ID | SR | NO: 42) | 43) | | |
| | NO: 40) | L | | | | |
| | | (SEQ ID | | | | |
| | | NO: 41) | | | | |
| TY23562 | YSIS | IGRI | AREF | RASQ | DASN | YCQQ |
| | SGYH | NPNR | GAY | SVDF | LETG | RA |
| | WDWI | GST | (SEQ ID | | V | SWPL |
| | (SEQ | KYAQ | NO: 48) | VGKS | (SEQ ID | T |
| | ID NO: | KFQG | | FLD | NO: 50) | (SEQ ID |
| | 46) | RV | | (SEQ ID | | NO: 51) |
| | | (SEQ ID | | NO: 49) | | |
| | | NO: 47) | | | | |

TABLE 2
HVR sequences of affinity-matured anti-CD137L Pab fragments

| Fab Name | HVR-H1 | HVR-H2 | HVR-H3 | HVR-L1 | HVR-L2 | HVR-L3 |
|---|---|---|---|---|---|---|
| Fab8949 | FSLN | IGLI | ARYG | RASQ | AAST | YCQQ |
| | TAGV | DWAG | YGGY | SVRG | LQSG | YSSL |
| | GVTW | YTYY | AIDY | SYLA | V | WT |
| | I | SPSL | (SEQ ID | (SEQ ID | (SEQ ID | (SEQ ID |
| | (SEQ ID | KSRL | (NO: 164) | (NO: 212) | (NO: 228) | (NO: 232) |
| | (NO: 68) | (SEQ ID | | | | |
| | | (NO: 116) | | | | |
| Fab8950 | FSLS | LAVI | ARYG | RASQ | AAST | YCQQ |
| | TSGV | DWAG | YRNY | SVRG | LQSG | YGSL |
| | GVAW | YKYY | ALDY | SYLA | V | WT |
| | I | SPSL | (SEQ ID | (SEQ ID | (SEQ ID | (SEQ ID |
| | (SEQ ID | KSRL | (NO: 165) | (NO: 212) | (NO: 228) | (NO: 233) |
| | (NO: 69) | (SEQ ID | | | | |
| | | (NO: 117) | | | | |
| Fab8954 | FSLS | IAVI | ARYG | RASQ | AAST | YCQQ |
| | TAGV | DWAG | YRNY | SVRG | LQSG | YGSL |
| | GVSW | AKYY | ALDY | NYLA | V | WT |
| | I | NPSL | (SEQ ID | (SEQ ID | (SEQ ID | (SEQ ID |
| | (SEQ ID | KSRL | (NO: 166) | (NO: 213) | (NO: 228) | (NO: 233) |
| | (NO: 70) | (SEQ ID | | | | |
| | | (NO: 118) | | | | |
| Fab8959 | FSLS | LAVI | ARYG | RASQ | AAST | YCQQ |
| | TSGV | DWAG | YRNY | SVRG | LQSG | YGSY |
| | GVAW | YKYY | ALDY | SYLA | V | WT |
| | I | SPSL | (SEQ ID | (SEQ ID | (SEQ ID | (SEQ ID |
| | (SEQ ID | KSRL | (NO: 167) | (NO: 212) | (NO: 228) | (NO: 234) |
| | (NO: 71) | (SEQ ID | | | | |
| | | (NO: 119) | | | | |
| Fab8963 | FSLS | IALI | ARYG | RASQ | DASS | YCQQ |
| | TSGV | DYAG | YRSY | SVRG | LESG | YSSY |
| | GVGW | SKYY | AMDY | NYLA | V | WT |
| | I | SPSL | (SEQ ID | (SEQ ID | (SEQ ID | (SEQ ID |
| | (SEQ ID | KSRL | (NO: 168) | (NO: 213) | (NO: 229) | (NO: 235) |
| | (NO: 72) | (SEQ ID | | | | |
| | | (NO: 120) | | | | |
| Fab8964 | FSLS | LGLI | ARYG | RASQ | DASS | YCQQ |
| | TGGV | DWAG | YGSY | SVRG | LESG | YSSL |
| | GVAW | YTYY | ALDY | SYLA | V | WT |
| | I | SPSL | (SEQ ID | (SEQ ID | (SEQ ID | (SEQ ID |
| | (SEQ ID | KSRL | (NO: 169) | (NO: 212) | (NO: 229) | (NO: 232) |
| | (NO: 73) | (SEQ ID | | | | |
| | | (NO: 121) | | | | |
| Fab8966 | FSLS | IGLI | ARYG | RASQ | DASS | YCQQ |
| | TGGV | DWAG | YRSY | SVSG | LESG | YNSS |
| | GVAW | YTYY | AMDY | SYLA | V | WT |
| | I | SPSL | (SEQ ID | (SEQ ID | (SEQ ID | (SEQ ID |
| | (SEQ ID | KSRL | (NO: 170) | (NO: 214) | (NO: 229) | (NO: 236) |
| | (NO: 74) | (SEQ ID | | | | |
| | | (NO: 122) | | | | |
| Fab8969 | FSLS | LAL1DWAG | ARYG | RASQ | DASS | YCQQ |
| | TAGV | DKYY | VGSY | T1RGSY | LESG | YSSL |
| | GVTW | SPSL | ALDY | LA | V | WT |
| | I | KSRL | (SEQ ID | (SEQ ID | (SEQ ID | (SEQ ID |
| | (SEQ ID | (SEQ ID | (NO: 171) | (NO: 215) | (NO: 229) | (NO: 232) |
| | (NO: 75) | (NO: 123) | | | | |
| Fab8971 | FSLS | LALI | ARYG | RASQ | DASS | YCQQ |
| | TAGV | DWAG | YSSY | SVRG | LESG | YSSY |
| | GVAW | DKYY | ALDY | SYLA | V | WT |
| | I | SPSL | (SEQ ID | (SEQ ID | (SEQ ID | (SEQ ID |
| | (SEQ ID | KSRL | (NO: 172) | (NO: 212) | (NO: 229) | (NO: 235) |
| | (NO: 76) | (SEQ ID | | | | |
| | | (NO: 124) | | | | |
| Fab8974 | FSLT | IGLI | ARYG | RASQ | DASS | YCQQ |
| | TTGV | DYAG | YDSY | SVRG | LESG | YSSY |
| | GVGW | YTYY | AMDY | NYLA | V | WT |
| | I | SPSL | (SEQ ID | (SEQ ID | (SEQ ID | (SEQ ID |
| | (SEQ ID | KSRL | (NO: 173) | (NO: 213) | (NO: 229) | (NO: 235) |
| | (NO: 77) | (SEQ ID | | | | |
| | | (NO: 125) | | | | |
| Fab8976 | FSLT | LGLI | ARYG | RASQ | DASS | YCQQ |
| | TSGV | DWSG | YRNY | SVRN | LESG | YSSS |
| | GVSW | YTYY | ALDY | SYLA | V | WT |
| | I | NPSL | (SEQ ID | (SEQ ID | (SEQ ID | (SEQ ID |
| | (SEQ ID | KSRL | (NO: 174) | (NO: 216) | (NO: 229) | (NO: 237) |
| | (NO: 78) | (SEQ ID | | | | |
| | | (NO: 126) | | | | |
| Fab8985 | FSLS | LALI | ARYG | RASQ | DASS | YCQQ |
| | TGGV | DWAG | WSSY | TVRG | LESG | YSSL |
| | GVGW | DKYY | ALDY | TFLA | V | WT |
| | I | SPSL | (SEQ ID | (SEQ ID | (SEQ ID | (SEQ ID |
| | (SEQ ID | KSRL | (NO: 175) | (NO: 217) | (NO: 229) | (NO: 232) |
| | (NO: 79) | (SEQ ID | | | | |
| | | (NO: 127) | | | | |
| Fab8986 | FSLS | IAVI | ARYG | RASQ | DASS | YCQQ |
| | TGGV | DYAG | YNSY | SVRG | LESG | YNSL |
| | GVSW | YTYY | ALDY | SYLA | V | WT |
| | I | SPSL | (SEQ ID | (SEQ ID | (SEQ ID | (SEQ ID |
| | (SEQ ID | KSRL | (NO: 176) | (NO: 212) | (NO: 229) | (NO: 238) |
| | (NO: 80) | (SEQ ID | | | | |
| | | (NO: 128) | | | | |
| Fab8996 | FSLS | LALI | ARYG | RASQ | DASS | YCQQ |
| | TGGV | DWAG | VSSY | TVRG | LESG | YSSL |
| | GVTW | DTYY | ALDY | TFLA | V | WT |
| | I | SPSL | (SEQ ID | (SEQ ID | (SEQ ID | (SEQ ID |
| | (SEQ ID | KSRL | (NO: 177) | (NO: 217) | (NO: 229) | (NO: 232) |
| | (NO: 8I) | (SEQ ID | | | | |
| | | (NO: 129) | | | | |
| Fab9010 | FSLT | LGLI | ARYG | RASQ | DASS | YCQQ |
| | TGGV | DWSG | YDSY | SVRG | LESG | YSSL |
| | GVTW | DKYY | AMDY | SYLA | V | WT |
| | I | NPSL | (SEQ ID | (SEQ ID | (SEQ ID | (SEQ ID |
| | (SEQ ID | KSRL | (NO: 178) | (NO: 212) | (NO: 229) | (NO: 232) |
| | (NO: 82) | (SEQ ID | | | | |
| | | (NO: 130) | | | | |
| Fab9011 | FSLT | IGL1DWAG | ARYG | RASQ | DASS | YCQQ |
| | TAGV | YKYY | YDGY | SVRG | LESG | YSSS |
| | GVAW | SPSL | AMDY | NYLA | V | WT |
| | I | KSRL | (SEQ ID | (SEQ ID | (SEQ ID | (SEQ ID |
| | (SEQ ID | (SEQ ID | (NO: 179) | (NO: 213) | (NO: 229) | (NO: 237) |
| | (NO: 83) | (NO: 131) | | | | |
| Fab9012 | FSLS | LALI | ARYG | RASQ | DASS | YCQQ |
| | TAGV | DWAG | YSSY | SVRG | LESG | YSSW |
| | GVTW | DKYY | ALDY | TFLA | V | WT |
| | I | SPSL | (SEQ ID | (SEQ ID | (SEQ ID | (SEQ ID |
| | (SEQ ID | KSRL | (NO: 180) | (NO: 218) | (NO: 229) | (NO: 239) |
| | (NO: 84) | (SEQ ID | | | | |
| | | (NO: 132) | | | | |
| Fab9013 | FSLN | IGLI | ARYG | RASQ | DASS | YCQQ |
| | TTGV | DWAG | YSGY | SVRG | LESG | YSSL |
| | GVSW | YTYY | ALDY | SYLA | V | WT |
| | I | NPSL | (SEQ ID | (SEQ ID | (SEQ ID | (SEQ ID |
| | (SEQ ID | KSRL | (NO: 181) | (NO: 212) | (NO: 229) | (NO: 232) |
| | (NO: 85) | (SEQ ID | | | | |
| | | (NO: 133) | | | | |
| Fab9014 | FSLS | IGLI | ARYG | RASQ | DASS | YCQQ |
| | TAGV | DWAG | YSGY | SVRG | LESG | YSSS |
| | GVAW | SKYY | AMDY | SYLA | V | WT |
| | I | NPSL | (SEQ ID | (SEQ ID | (SEQ ID | (SEQ ID |
| | (SEQ ID | KSRL | (NO: 182) | (NO: 212) | (NO: 229) | (NO: 237) |
| | (NO: 86) | (SEQ ID | | | | |
| | | (NO: 134) | | | | |
| Fab9015 | FSLT | LAVI | ARYG | RASQ | DASS | YCQQ |
| | TGGV | DWAG | YNSY | SVRG | LESG | YSSE |
| | GVAW | YKYY | ALDY | SYLA | V | WT |
| | I | SPSL | (SEQ ID | (SEQ ID | (SEQ ID | (SEQ ID |
| | (SEQ ID | KSRL | (NO: 183) | (NO: 212) | (NO: 229) | (NO: 240) |
| | (NO: 87) | (SEQ ID | | | | |
| | | (NO: 135) | | | | |
| Fab9016 | FSLS | LALI | ARYG | RASQ | DASS | YCQQ |
| | TGGV | DWAG | YGSY | TVRG | LESG | YSSL |
| | GVAW | DKYY | ALDY | TFLA | V | WT |
| | I | SPSL | (SEQ ID | (SEQ ID | (SEQ ID | (SEQ ID |
| | (SEQ ID | KSRL | (NO: 184) | (NO: 217) | (NO: 229) | (NO: 232) |
| | (NO: 88) | (SEQ ID | | | | |
| | | (NO: 136) | | | | |
| Fab9017 | FSLS | LALI | ARYG | RASQ | DASS | YCQQ |
| | TGGV | DWAG | YGSY | TVRG | LESG | YSSL |
| | GVGW | DKYY | ALDY | SYLA | V | WT |
| | I | SPSL | (SEQ ID | (SEQ ID | (SEQ ID | (SEQ ID |
| | (SEQ ID | KSRL | (NO: 185) | (NO: 219) | (NO: 229) | (NO: 232) |
| | (NO: 89) | (SEQ ID | | | | |
| | | (NO: 137) | | | | |
| Fab9018 | FSLN | IGLI | ARYG | RASQ | DASS | YCQQ |
| | TGGV | DWAG | YDSY | SVRG | LESG | YSSS |
| | GVTW | YKYY | ALDY | NYLA | V | WT |
| | I | SPSL | (SEQ ID | (SEQ ID | (SEQ ID | (SEQ ID |
| | (SEQ ID | KSRL | (NO: 186) | (NO: 213) | (NO: 229) | (NO: 237) |
| | (NO: 90) | (SEQ ID | | | | |
| | | (NO: 138) | | | | |
| Fab9019 | FSLS | LALI | ARYG | RASQ | AAST | YCQQ |
| | TAGV | DWAD | YSNY | TVRG | LQSG | YGSL |
| | GVAW | DKYY | ALDY | NYLA | V | WT |
| | I | SPSL | (SEQ ID | (SEQI | (SEQ ID | (SEQ ID |
| | (SEQ ID | KSRL | (NO: 187) | DNG:220) | (NO: 228) | (NO: 233) |
| | (NO: 9I) | (SEQ ID | | | | |
| | | (NO: 139) | | | | |
| Fab9020 | FSLS | LALI | ARYG | RASQ | DASS | YCQQ |
| | TAGV | DWAG | LSSY | TVRG | LESG | YSSL |
| | GVAW | DTYY | ALDY | TFLA | V | WT |
| | I | SPSL | (SEQ ID | (SEQ ID | (SEQ ID | (SEQ ID |
| | (SEQ ID | KSRL | (NO: 188) | (NO: 217) | (NO: 229) | (NO: 232) |
| | (NO: 92) | (SEQ ID | | | | |
| | | (NO: 140) | | | | |
| Fab9022 | FSLS | LALI | ARYG | RASQ | DASS | YCQQ |
| | TAGV | DYAG | YSGY | SVRG | LESG | YSSS |
| | GVAW | DKYY | ALDY | NYLA | V | WT |
| | I | NPSP | (SEQ ID | (SEQ ID | (SEQ ID | (SEQ ID |
| | (SEQ ID | KSHL | (NO: 189) | (NO: 213) | (NO: 229) | (NO: 237) |
| | (NO: 93) | (SEQ ID | | | | |
| | | (NO: !41) | | | | |
| Fab9023 | FSLS | LAVI | ARYG | RASQ | DASS | YCQQ |
| | TGGV | DYAG | YGSY | SVRG | LESG | YSSS |
| | GVGW | YTYY | ALDY | SYLA | V | WT |
| | I | SPSL | (SEQ ID | (SEQ ID | (SEQ ID | (SEQ ID |
| | (SEQ ID | KSRL | (NO: 190) | (NO: 212) | (NO: 229) | (NO: 237) |
| | (NO: 94) | (SEQ ID | | | | |
| | | (NO: 142) | | | | |
| Fab9024 | FSLS | LALI | ARYG | RASQ | DASS | YCQQ |
| | TAGV | DYAG | YSSY | SVRG | LESG | YSSY |
| | GVGW | YTYY | ALDY | SYLA | V | WT |
| | I | SPSL | (SEQ ID | (SEQ ID | (SEQ ID | (SEQ ID |
| | (SEQ ID | KSRL | (NO: 191) | (NO: 212) | (NO: 229) | (NO: 235) |
| | (NO: 95) | . | | | | |
| | | (SEQ ID | | | | |
| | | (NO: 143) | | | | |
| Fab9025 | FSLS | LALI | ARYG | RASQ | DASS | YCQQ |
| | TSGV | DYAG | YSSY | SVRG | LESG | YSSS |
| | GVAW | YTYY | ALDY | SYLA | V | WT |
| | I | SPSL | (SEQ ID | (SEQ ID | (SEQ ID | (SEQ ID |
| | (SEQ ID | KSRL | (NO: 192) | (NO: 212) | (NO: 229) | (NO: 237) |
| | (NO: 96) | (SEQ ID | | | | |
| | | (NO: 144) | | | | |
| Fab9026 | FSLS | LALI | ARYG | RASQ | AAFT | YCQQ |
| | TGGV | DYAG | YSGY | SIRG | LQSG | YSSS |
| | GVSW | YTYY | ALDY | NYLA | V | WT |
| | I | SPSL | (SEQ ID | (SEQ ID | (SEQ ID | (SEQ ID |
| | (SEQ ID | KSRL | (NO: 193) | (NO: 221) | (NO: 230) | (NO: 237) |
| | (NO: 97) | (SEQ ID | | | | |
| | | (NO: 145) | | | | |
| Fab9027 | FSLS | LALI | ARYG | RTSQ | DASS | YCQQ |
| | TSGV | DYAG | YGSY | SVRG | LESG | YSSS |
| | GVTW | YKYY | ALDY | SYLA | V | WT |
| | I | SPSL | (SEQ ID | (SEQ ID | (SEQ ID | (SEQ ID |
| | (SEQ ID | KSRL | (NO: 194) | (NO: 222) | (NO: 229) | (NO: 237) |
| | (NO: 98) | (SEQ ID | | | | |
| | | (NO: 146) | | | | |
| Fab9028 | FSLS | LALI | ARYG | RASQ | DASS | YCQQ |
| | TGGV | DWSG | YGSY | SIRG | LESG | YS |
| | GVAW | DKYY | ALDY | SYLA | V | |
| | I | SPSL | (SEQ ID | (SEQ ID | (SEQ ID | |
| | (SEQ ID | KSRL | (NO: 195) | (NO: 223) | (NO: 229) | |
| | (NO: 99) | (SEQ ID | | | | |
| | | (NO: 147) | | | | SSWT |
| | | | | | | (SEQ ID |
| | | | | | | (NO: 237) |
| Fab9029 | FSLS | LALI | ARYG | RASQ | DASS | YCQQ |
| | TSGV | DYSG | YGSY | SIRG | LESG | YSSS |
| | GVAW | ATYY | ALDY | SYLA | V | WT |
| | I | SPSL | (SEQ ID | (SEQ ID | (SEQ ID | (SEQ ID |
| | (SEQ ID | KSRL | (NO: 196) | (NO: 223) | (NO: 229) | (NO: 237) |
| | (NO: 100) | (SEQ ID | | | | |
| | | (NO: 148) | | | | |
| Fab9030 | FSLS | LALI | ARYG | RASQ | DASS | YCQQ |
| | TSGV | DYAG | YSGY | SIHG | LESG | YSSS |
| | GVAW | YTYY | ALDY | SYLA | V | WT |
| | I | SPSL | (SEQ ID | (SEQI | (SEQ ID | (SEQ ID |
| | (SEQ ID | KSRL | (NO: 197) | DNQ:224) | (NO: 229) | (NO: 237) |
| | (NO: 101) | (SEQ ID | | | | |
| | | (NO: 149) | | | | |
| Fab9031 | FSLS | LALI | ARYG | RASQ | DASS | YCQQ |
| | TGGV | DYSG | YGSY | SVRG | LESG | YSSF |
| | GVAW | DKYY | ALDY | SYLA | V | WT |
| | I | SPSP | (SEQ ID | (SEQ ID | (SEQ ID | (SEQ ID |
| | (SEQ ID | KSRL | (NO: 198) | (NO: 212) | (NO: 229) | (NO: 240) |
| | (NO: 102) | (SEQ ID | | | | |
| | | (NO: 150) | | | | |
| Fab9032 | FSLS | LALI | ARYG | RASQ | DASS | YCQQ |
| | TAGV | DYAG | YGSY | SIRG | LESG | YSSL |
| | GVSW | SKYY | ALDY | SYLA | V | WT |
| | I | SPSL | (SEQ ID | (SEQ ID | (SEQ ID | (SEQ ID |
| | (SEQ ID | KSRL | (NO: 199) | (NO: 223) | (NO: 229) | (NO: 232) |
| | (NO: 103) | (SEQ ID | | | | |
| | | (NO: 151) | | | | |
| Fab9033 | FSLS | LALI | ARYG | RASQ | DASS | YCQQ |
| | TGGV | DWAG | YGSY | SIRG | LESG | YSSF |
| | GVAW | DKYY | ALDY | SYLA | V | WT |
| | I | NPSL | (SEQ ID | (SEQ ID | (SEQ ID | (SEQ ID |
| | (SEQ ID | KSRL | (NO: 200) | (NO: 223) | (NO: 229) | (NO: 240) |
| | (NO: 104) | (SEQ ID | | | | |
| | | (NO: 152) | | | | |
| Fab9036 | FSLS | LALI | ARYG | RASQ | DASS | YCQQ |
| | TGGV | DYAG | YGGY | SVRG | LESG | YSSF |
| | GVAW | YKYY | ALDY | NYLA | V | WT |
| | I | SPSL | (SEQ ID | (SEQ ID | (SEQ ID | (SEQ ID |
| | (SEQ ID | KSRL | (NO: 201) | (NO: 213) | (NO: 229) | (NO: 24Q) |
| | (NO: 105) | .(SEQI | | | | |
| | | DNOT | | | | |
| | | 53) | | | | |
| Fab9037 | FSLS | LAII | ARYG | RASQ | DASS | YCQQ |
| | TTGV | DWSG | YSSY | SVHG | LESG | YNSL |
| | GVAW | 5KYYS | ALDY | SYLA | V | WT |
| | I | PSLK | (SEQ ID | (SEQ ID | (SEQ ID | (SEQ ID |
| | (SEQ ID | SRL | (NO: 202) | (NO: 225) | (NO: 229) | (NO: 238) |
| | (NO: 106) | (SEQ ID | | | | |
| | | (NO: 154) | | | | |
| Fab9038 | FSLS | LALI | ARYG | RASQ | DASS | YCQQ |
| | TAGV | DWSG | YSGY | GVSS | LESG | YGSY |
| | GVGW | ATYY | ALDY | YLA | V | WT |
| | I | SPSL | (SEQ ID | (SEQ ID | (SEQ ID | (SEQ ID |
| | (SEQ ID | KSRL | (NO: 203) | (NO: 226) | (NO: 229) | (NO: 234) |
| | (NO: 107) | (SEQ ID | | | | |
| | | (NO: 155) | | | | |
| Fab9039 | FSLS | LALI | ARYG | RASQ | DASS | YCQQ |
| | TAGV | DYAG | YSGY | SIRG | LESG | YSSS |
| | GVAW | DKYY | ALDY | NYLA | V | WT |
| | I | NPSP | (SEQ ID | (SEQ ID | (SEQ ID | (SEQ ID |
| | (SEQ ID | KSHL | (NO: 204) | (NO: 221) | (NO: 229) | (NO: 237) |
| | (NO: 108) | (SEQ ID | | | | |
| | | (NO: 156) | | | | |
| Fab8955 | FSLS | LALI | ARYG | RASQ | DASS | YCQQ |
| | TG | DWAG | YGSY | TVRG | LES | YS |
| | | DK | AL | | | |
| | GVGV | YYSP | DY | SYLA | GV | SLWT |
| | SWI | SLKS | (SEQ ID | (SEQ ID | (SEQ ID | (SEQ ID |
| | (SEQ ID | RL | NO: 205) | NO: 219) | NO: 229) | NO: 232) |
| | NO: 109) | (SEQ ID | | | | |
| | | NO: 157) | | | | |
| Fab8957 | FSLS | LALI | ARYG | RASQ | DASS | YCQQ |
| | TAGV | DWAG | VSSY | TVRG | LESG | YSSL |
| | GVSW | DTYY | ALDY | TYLA | V | WT |
| | I | SPSL | (SEQ ID | (SEQ ID | (SEQ ID | (SEQ ID |
| | (SEQ ID | KSRL | NO: 206) | NO: 227) | NO: 229) | NO: 232) |
| | NO: 110) | (SEQ ID | | | | |
| | | NO: 158) | | | | |
| Fab8958 | FSLS | LALI | ARYG | RASQ | DASS | YCQQ |
| | TGGV | DWAG | GSSY | TVRG | LESG | YSSL |
| | GVAW | DKYY | ALDY | TFLA | V | WT |
| | I | SPSL | (SEQ ID | (SEQ ID | (SEQ ID | (SEQ ID |
| | (SEQ ID | KSRL | NO: 207) | NO: 217) | NO: 229) | NO: 232) |
| | NO: III) | (SEQ ID | | | | |
| | | NO: 159) | | | | |
| Fab8947 | FSLN | LALI | ARYG | RASQ | AASS | YCQQ |
| | TAGV | DWAG | YRNY | SVRG | LQSG | YSSY |
| | GVSW | YKYY | AMDY | NYLA | V | WT |
| | I | SPSL | (SEQ ID | (SEQ ID | (SEQ ID | (SEQ ID |
| | (SEQ ID | KSRL | NO: 208) | NO: 213) | NO: 231) | NO: 235) |
| | NO: 112) | (SEQ ID | | | | |
| | | NO: 160) | | | | |
| Fab8948 | FSLT | IGLI | ARYG | RASQ | DASS | YCQQ |
| | TSGV | DHAG | YDSY | SVRG | LESG | YSSS |
| | GVAW | YTYY | AMDY | SYLA | V | WT |
| | I | SPSL | (SEQ ID | (SEQ ID | (SEQ ID | (SEQ ID |
| | (SEQ ID | KSRL | NO: 209) | NO: 212) | NO: 229) | NO: 237) |
| | NO: 113) | (SEQ ID | | | | |
| | | NO: 161) | | | | |
| Fab8953 | FSLT | IGLI | ARYG | RASQ | DASS | YCQQ |
| | TSGV | DYAG | YRGY | SVRG | LESG | YSSF |
| | GVGW | YKYY | ALDY | NYLA | V | WT |
| | I | SPSL | (SEQ ID | (SEQ ID | (SEQ ID | (SEQ ID |
| | (SEQ ID | KSRL | NO: 210) | NO: 213) | NO: 229) | NO: 240) |
| | NO: 114) | (SEQ ID | | | | |
| | | NO: 162) | | | | |
| Fab8952 | FSLS | LALI | ARYG | RASQ | DASS | YCQQ |
| | TAGV | DWAG | VGSY | TVRG | LESG | YSSL |
| | GVAW | DTYY | ALDY | TFLA | V | WT |
| | I | SPSL | (SEQ ID | (SEQ ID | (SEQ ID | (SEQ ID |
| | (SEQ ID | KSRL | NO: 211) | NO: 217) | NO: 229) | NO: 232) |
| | NO: 115) | (SEQ ID | | | | |
| | | NO: 163) | | | | |

In some embodiments, the anti-CD137L antibody comprises 1, 2, 3, 4, 5, or 6 HVR sequences of a single antibody listed in Table 1 or Table 2, comprises 1, 2, 3, 4, 5, or 6 HVR sequences of a V_{H} and/or V_{L} domain of a single antibody listed in Table 3 or Table 4.

In some embodiments, the anti-CD137L antibody comprises an HVR-H1 comprising the amino acid sequence of SEQ ID NO:4, an HVR-H12 comprising the amino acid sequence of SEQ ID NO: 5, an HVR-H3 comprising the amino acid sequence of SEQ ID NO: 6, an HVR-LI comprising the amino acid sequence of SEQ ID NO: 7, an HVR-L2 comprising the amino acid sequence of SEQ ID NO: 8, and an HVR-L3 comprising the amino acid sequence of SEQ ID NO:9. In some embodiments, the anti-CD137L antibody comprises an HVR-H1 comprising the amino acid sequence of SEQ ID NO:10, an HVR-H2 comprising the amino acid sequence of SEQ ID NO:11, an HVR-H3 comprising the amino acid sequence of SEQ ID NO:12, an HVR-L1 comprising the amino acid sequence of SEQ ID NO:13, an HVR-L2 comprising the amino acid sequence of SEQ ID NO:14, and an HVR-L3 comprising the amino acid sequence of SEQ ID NO:15. In some embodiments, the anti-CD137L antibody comprises an HVR-H1 comprising the amino acid sequence of SEQ ID NO:16, an HVR-H2 comprising the amino acid sequence of SEQ ID NO:17, an HVR-H3 comprising the amino acid sequence of SEQ ID NO:18, an HVR-L1 comprising the amino acid sequence of SEQ ID NO:19, an HVR-L2 comprising the amino acid sequence of SEQ ID NO:20, and an HVR-L3 comprising the amino acid sequence of SEQ ID NO:21. In some embodiments, the anti-CD137L antibody comprises an HVR-H1 comprising the amino acid sequence of SEQ ID NO:22, an HVR-H2 comprising the amino acid sequence of SEQ ID NO:23, an HVR-H3 comprising the amino acid sequence of SEQ ID NO:24, an HVR-L1 comprising the amino acid sequence of SEQ ID NO:25, an HVR-L2 comprising the amino acid sequence of SEQ ID NO:26, and an HVR-L3 comprising the amino acid sequence of SEQ ID NO:27. In some embodiments, the anti-CD137L antibody comprises an HVR-H1 comprising the amino acid sequence of SEQ ID NO:28, an HVR-H2 comprising the amino acid sequence of SEQ ID NO:29, an HVR-H3 comprising the amino acid sequence of SEQ ID NO:30, an HVR-L1 comprising the amino acid sequence of SEQ ID NO:31, an HVR-L2 comprising the amino acid sequence of SEQ ID NO:32, and an HVR-L3 comprising the amino acid sequence of SEQ ID NO:33. In some embodiments, the anti-CD137L antibody comprises an HVR-H1 comprising the amino acid sequence of SEQ ID NO:34, an HVR-H2 comprising the amino acid sequence of SEQ ID NO:35, an HVR-H3 comprising the amino acid sequence of SEQ ID NO:36, an HVR-L1 comprising the amino acid sequence of SEQ ID NO:37, an HVR-L2 comprising the amino acid sequence of SEQ ID NO:38, and an HVR-L3 comprising the amino acid sequence of SEQ ID NO:39. In some embodiments, the anti-CD137L antibody comprises an HVR-H1 comprising the amino acid sequence of SEQ ID NO:40, an HVR-H2 comprising the amino acid sequence of SEQ ID NO:41, an HVR-H3 comprising the amino acid sequence of SEQ ID NO:42, an HVR-L1 comprising the amino acid sequence of SEQ ID NO:43, an HVR-L2 comprising the amino acid sequence of SEQ ID NO:4, and an HVR-L3 comprising the amino acid sequence of SEQ ID NO:45. In some embodiments, the anti-CD137L antibody comprises an HVR-H1 comprising the amino acid sequence of SEQ ID NO:46, an HVR-H2 comprising the amino acid sequence of SEQ ID NO:47, an HVR-H3 comprising the amino acid sequence of SEQ ID NO:48, an HVR-L1 comprising the amino acid sequence of SEQ ID NO:49, an HVR-L2 comprising the amino acid sequence of SEQ ID NO:50, and an HVR-L3 comprising the amino acid sequence of SEQ ID NO:51.

In some embodiments, the anti-CD137L antibody comprises an HVR-H1 comprising an amino acid sequence selected from the group consisting of SEQ ID NOs:68-115, an HVR-H2 comprising an amino acid sequence selected from the group consisting of SEQ ID NOs:116-163, an HVR-H3 comprising an amino acid sequence selected from the group consisting of SEQ ID NOs:164-211, an HVR-L1 comprising an amino acid sequence selected from the group consisting of SEQ ID NOs:212-227, an HVR-L2 comprising an amino acid sequence selected from the group consisting of SEQ ID NOs:228-231, and an HVR-L3 comprising an amino acid sequence selected from the group consisting of SEQ ID NOs:232-240.

In some embodiments, the anti-CD137L antibody comprises an HVR-H1 comprising the amino acid sequence of SEQ ID NO:68, an HVR-H2 comprising the amino acid sequence of SEQ ID NO:116, an HVR-H3 comprising the amino acid sequence of SEQ ID NO:164, an HVR-L1 comprising the amino acid sequence of SEQ ID NO:212, an HVR-L2 comprising the amino acid sequence of SEQ ID NO:228, and an HVR-L3 comprising the amino acid sequence of SEQ ID NO:232. In some embodiments, the anti-CD137L antibody comprises an HVR-H1 comprising the amino acid sequence of SEQ ID NO:69, an HVR-H2 comprising the amino acid sequence of SEQ ID NO:117, an HVR-H3 comprising the amino acid sequence of SEQ ID NO:165, an HVR-L1 comprising the amino acid sequence of SEQ ID NO:212, an HVR-L2 comprising the amino acid sequence of SEQ ID NO:228, and an HVR-L3 comprising the amino acid sequence of SEQ ID NO:233. In some embodiments, the anti-CD137L antibody comprises an HVR-H1 comprising the amino acid sequence of SEQ ID NO:70, an HVR-H2 comprising the amino acid sequence of SEQ ID NO:118, an HVR-H3 comprising the amino acid sequence of SEQ ID NO:166, an HVR-L1 comprising the amino acid sequence of SEQ ID NO:213, an HVR-L2 comprising the amino acid sequence of SEQ ID NO:228, and an HVR-L3 comprising the amino acid sequence of SEQ ID NO:233. In some embodiments, the anti-CD137L antibody comprises an HVR-H1 comprising the amino acid sequence of SEQ ID NO:71, an HVR-H2 comprising the amino acid sequence of SEQ ID NO:119, an HVR-H3 comprising the amino acid sequence of SEQ ID NO:167, an HVR-L1 comprising the amino acid sequence of SEQ ID NO:212, an HVR-L2 comprising the amino acid sequence of SEQ ID NO:228, and an HVR-L3 comprising the amino acid sequence of SEQ ID NO:234. In some embodiments, the anti-CD137L antibody comprises an HVR-H1 comprising the amino acid sequence of SEQ ID NO:72, an HVR-H2 comprising the amino acid sequence of SEQ ID NO:120, an HVR-H3 comprising the amino acid sequence of SEQ ID NO:168, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:235. In some embodiments, the anti-CD137L antibody comprises an HVR-H1 comprising the amino acid sequence of SEQ ID NO:73, an HVR-H2 comprising the amino acid sequence of SEQ ID NO:121, an HVR-H3 comprising the amino acid sequence of SEQ ID NO:169, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:74, an HVR-H2 comprising the amino acid sequence of SEQ ID NO:122, an HVR-H3 comprising the amino acid sequence of SEQ ID NO:170, an HVR-L1 comprising the amino acid sequence of SEQ ID NO:214, an HVR-L2 comprising the amino acid sequence of SEQ ID NO:229, and an HVR-L3 comprising the amino acid sequence of SEQ ID NO:236. In some embodiments, the anti-CD137L antibody comprises an HVR-H1 comprising the amino acid sequence of SEQ ID NO:75, an HVR-H2 comprising the amino acid sequence of SEQ ID NO:123, an HVR-H3 comprising the amino acid sequence of SEQ ID NO:171, an HVR-L1 comprising the amino acid sequence of SEQ ID NO:215, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:76, an HVR-H2 comprising the amino acid sequence of SEQ ID NO:124, an HVR-H3 comprising the amino acid sequence of SEQ ID NO:172, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:235. In some embodiments, the anti-CD137L antibody comprises an HVR-H1 comprising the amino acid sequence of SEQ ID NO:77, an HVR-H2 comprising the amino acid sequence of SEQ ID NO:125, an HVR-H3 comprising the amino acid sequence of SEQ ID NO:173, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:235. In some embodiments, the anti-CD137L antibody comprises an HVR-H1 comprising the amino acid sequence of SEQ ID NO:78, an HVR-H2 comprising the amino acid sequence of SEQ ID NO:126, an HVR-H3 comprising the amino acid sequence of SEQ ID NO:174, an HVR-LI comprising the amino acid sequence of SEQ ID NO:216, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:79, an HVR-H2 comprising the amino acid sequence of SEQ ID NO:127, an HVR-H3 comprising the amino acid sequence of SEQ ID NO:175, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:80, an HVR-H2 comprising the amino acid sequence of SEQ ID NO:128, an HVR-H3 comprising the amino acid sequence of SEQ ID NO:176, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:238. In some embodiments, the anti-CD137L antibody comprises an HVR-H1 comprising the amino acid sequence of SEQ ID NO:81, an HVR-H2 comprising the amino acid sequence of SEQ ID NO:129, an HVR-H3 comprising the amino acid sequence of SEQ ID NO:177, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:82, an HVR-H2 comprising the amino acid sequence of SEQ ID NO:130, an HVR-H3 comprising the amino acid sequence of SEQ ID NO:178, an HVR-LI comprising the amino acid sequence of SEQ ID NO:212, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:83, an HVR-H2 comprising the amino acid sequence of SEQ ID NO:131, an HVR-H3 comprising the amino acid sequence of SEQ ID NO:179, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:84, an HVR-H2 comprising the amino acid sequence of SEQ ID NO:132, an HVR-H3 comprising the amino acid sequence of SEQ ID NO:180, an HVR-L1 comprising the amino acid sequence of SEQ ID NO:218, an HVR-L2 comprising the amino acid sequence of SEQ ID NO:229, and an HVR-L3 comprising the amino acid sequence of SEQ ID NO:239. In some embodiments, the anti-CD137L antibody comprises an HVR-H1 comprising the amino acid sequence of SEQ ID NO:85, an HVR-H2 comprising the amino acid sequence of SEQ ID NO:133, an HVR-H3 comprising the amino acid sequence of SEQ ID NO:181, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:86, an HVR-H2 comprising the amino acid sequence of SEQ ID NO:134, an HVR-H3 comprising the amino acid sequence of SEQ ID NO:182, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:87, an HVR-H2 comprising the amino acid sequence of SEQ ID NO:135, an HVR-H3 comprising the amino acid sequence of SEQ ID NO:183, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:240. In some embodiments, the anti-CD137L antibody comprises an HVR-H1 comprising the amino acid sequence of SEQ ID NO:88, an HVR-H2 comprising the amino acid sequence of SEQ ID NO:136, an HVR-H3 comprising the amino acid sequence of SEQ ID NO:184, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:89, an HVR-H2 comprising the amino acid sequence of SEQ ID NO:137, an HVR-H3 comprising the amino acid sequence of SEQ ID NO:185, an HVR-L1 comprising the amino acid sequence of SEQ ID NO:219, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:90, an HVR-H2 comprising the amino acid sequence of SEQ ID NO:138, an HVR-H3 comprising the amino acid sequence of SEQ ID NO:186, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:91, an HVR-H2 comprising the amino acid sequence of SEQ ID NO:139, an HVR-H3 comprising the amino acid sequence of SEQ ID NO:187, an HVR-L1 comprising the amino acid sequence of SEQ ID NO:220, an HVR-L2 comprising the amino acid sequence of SEQ ID NO:228, and an HVR-L3 comprising the amino acid sequence of SEQ ID NO:233. In some embodiments, the anti-CD137L antibody comprises an HVR-H1 comprising the amino acid sequence of SEQ ID NO:92, an HVR-H2 comprising the amino acid sequence of SEQ ID NO:140, an HVR-H3 comprising the amino acid sequence of SEQ ID NO:188, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:93, an HVR-H2 comprising the amino acid sequence of SEQ ID NO:141, an HVR-H3 comprising the amino acid sequence of SEQ ID NO:189, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:94, an HVR-H2 comprising the amino acid sequence of SEQ ID NO:142, an HVR-H3 comprising the amino acid sequence of SEQ ID NO:190, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:95, an HVR-H2 comprising the amino acid sequence of SEQ ID NO:143, an HVR-H3 comprising the amino acid sequence of SEQ ID NO:191, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:235. In some embodiments, the anti-CD137L antibody comprises an HVR-H1 comprising the amino acid sequence of SEQ ID NO:96, an HVR-H2 comprising the amino acid sequence of SEQ ID NO:144, an HVR-H3 comprising the amino acid sequence of SEQ ID NO:192, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:97, an HVR-H2 comprising the amino acid sequence of SEQ ID NO:145, an HVR-H3 comprising the amino acid sequence of SEQ ID NO:193, an HVR-L1 comprising the amino acid sequence of SEQ ID NO:221, an HVR-L2 comprising the amino acid sequence of SEQ ID NO:230, and an HVR-L3 comprising the amino acid sequence of SEQ ID NO:237. In some embodiments, the anti-CD137L antibody comprises an HVR-H1 comprising the amino acid sequence of SEQ ID NO:98, an HVR-H2 comprising the amino acid sequence of SEQ ID NO:146, an HVR-H3 comprising the amino acid sequence of SEQ ID NO:194, an HVR-L1 comprising the amino acid sequence of SEQ ID NO:222, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:99, an HVR-H2 comprising the amino acid sequence of SEQ ID NO:147, an HVR-H3 comprising the amino acid sequence of SEQ ID NO:195, an HVR-L1 comprising the amino acid sequence of SEQ ID NO:223, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:100, an HVR-H2 comprising the amino acid sequence of SEQ ID NO:148, an HVR-H3 comprising the amino acid sequence of SEQ ID NO:196, an HVR-L1 comprising the amino acid sequence of SEQ ID NO:223, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:101, an HVR-H2 comprising the amino acid sequence of SEQ ID NO:149, an HVR-H3 comprising the amino acid sequence of SEQ ID NO:197, an HVR-L1 comprising the amino acid sequence of SEQ ID NO:224, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:102, an HVR-H2 comprising the amino acid sequence of SEQ ID NO:150, an HVR-H3 comprising the amino acid sequence of SEQ ID NO:198, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:240. In some embodiments, the anti-CD137L antibody comprises an HVR-H1 comprising the amino acid sequence of SEQ ID NO:103, an HVR-H2 comprising the amino acid sequence of SEQ ID NO:151, an HVR-H3 comprising the amino acid sequence of SEQ ID NO:199, an HVR-L1 comprising the amino acid sequence of SEQ ID NO:223, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:104, an HVR-H2 comprising the amino acid sequence of SEQ ID NO:152, an HVR-H3 comprising the amino acid sequence of SEQ ID NO:200, an HVR-L1 comprising the amino acid sequence of SEQ ID NO:223, an HVR-L2 comprising the amino acid sequence of SEQ ID NO:229, and an HVR-L3 comprising the amino acid sequence of SEQ ID NO:240. In some embodiments, the anti-CD137L antibody comprises an HVR-H1 comprising the amino acid sequence of SEQ ID NO:105, an HVR-H2 comprising the amino acid sequence of SEQ ID NO:153, an HVR-H3 comprising the amino acid sequence of SEQ ID NO:201, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:240. In some embodiments, the anti-CD137L antibody comprises an HVR-H1 comprising the amino acid sequence of SEQ ID NO:106, an HVR-H2 comprising the amino acid sequence of SEQ ID NO:154, an HVR-H3 comprising the amino acid sequence of SEQ ID NO:202, an HVR-L1 comprising the amino acid sequence of SEQ ID NO:225, an HVR-L2 comprising the amino acid sequence of SEQ ID NO:229, and an HVR-L3 comprising the amino acid sequence of SEQ ID NO:238. In some embodiments, the anti-CD137L antibody comprises an HVR-H1 comprising the amino acid sequence of SEQ ID NO:107, an HVR-H2 comprising the amino acid sequence of SEQ ID NO:155, an HVR-H3 comprising the amino acid sequence of SEQ ID NO:203, an HVR-L1 comprising the amino acid sequence of SEQ ID NO:226, an HVR-L2 comprising the amino acid sequence of SEQ ID NO:229, and an HVR-L3 comprising the amino acid sequence of SEQ ID NO:234. In some embodiments, the anti-CD137L antibody comprises an HVR-H1 comprising the amino acid sequence of SEQ ID NO:108, an HVR-H2 comprising the amino acid sequence of SEQ ID NO:156, an HVR-H3 comprising the amino acid sequence of SEQ ID NO:204, an HVR-L1 comprising the amino acid sequence of SEQ ID NO:221, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:109, an HVR-H2 comprising the amino acid sequence of SEQ ID NO:157, an HVR-H3 comprising the amino acid sequence of SEQ ID NO:205, an HVR-L1 comprising the amino acid sequence of SEQ ID NO:219, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:110, an HVR-H2 comprising the amino acid sequence of SEQ ID NO:158, an HVR-H3 comprising the amino acid sequence of SEQ ID NO:206, an HVR-L1 comprising the amino acid sequence of SEQ ID NO:227, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:111, an HVR-H2 comprising the amino acid sequence of SEQ ID NO:159, an HVR-H3 comprising the amino acid sequence of SEQ ID NO:207, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232. In some embodiments, the anti-CD137L antibody comprises an HVR-H1 comprising the amino acid sequence of SEQ ID NO:112, an HVR-H2 comprising the amino acid sequence of SEQ ID NO:160, an HVR-H3 comprising the amino acid sequence of SEQ ID NO:208, an HVR-LI comprising the amino acid sequence of SEQ ID NO:213, an HVR-L2 comprising the amino acid sequence of SEQ ID NO:231, and an HVR-L3 comprising the amino acid sequence of SEQ ID NO:235. In some embodiments, the anti-CD137L antibody comprises an HVR-H1 comprising the amino acid sequence of SEQ ID NO:113, an HVR-H2 comprising the amino acid sequence of SEQ ID NO:161, an HVR-H3 comprising the amino acid sequence of SEQ ID NO:209, an HVR-L1 comprising the amino acid sequence of SEQ ID NO:212, an HVR-L2 comprising the amino acid sequence of SEQ ID NO:229, and an HVR-L3 comprising the amino acid sequence of SEQ ID NO:237. In some embodiments, the anti-CD137L antibody comprises an HVR-H1 comprising the amino acid sequence of SEQ ID NO:114, an HVR-H2 comprising the amino acid sequence of SEQ ID NO:162, an HVR-H3 comprising the amino acid sequence of SEQ ID NO:210, an HVR-L1 comprising the amino acid sequence of SEQ ID NO:213, an HVR-L2 comprising the amino acid sequence of SEQ ID NO:229, and an HVR-L3 comprising the amino acid sequence of SEQ ID NO:240. In some embodiments, the anti-CD137L antibody comprises an HVR-H1 comprising the amino acid sequence of SEQ ID NO:115, an HVR-H2 comprising the amino acid sequence of SEQ ID NO:163, an HVR-H3 comprising the amino acid sequence of SEQ ID NO:211, an HVR-L1 comprising the amino acid sequence of SEQ ID NO:217, an HVR-L2 comprising the amino acid sequence of SEQ ID NO:229, and an HVR-L3 comprising the amino acid sequence of SEQ ID NO:232.

TABLE 3
VH and VL sequences of
anti-CD137L antibodies

| Antibody | | |
|---|---|---|
| Name | VH Sequence | VL sequence |
| TY23554 | EVQLVESGGGLVQPG | DIQLTQSPSSLSASV |
| | GSLRLSCAASGYSIT | GDRVTITCRASQDIS |
| | SGYYWAWIRQAPGKG | SDLAWYQQKPGKAPK |
| | LEWIGIINPNRGSTK | LLIYDASNLETGVPS |
| | YAQKFQGRVTISRDN | RFSGSGSGTDFTLTI |
| | SKNTLYLQLNSLRAE | SSLQPEDFATYYCQQ |
| | DTAVYYCARDGWALS | YDAWPSTFGQGTKVE |
| | GGYYYYYGLDVWGQG | IKR |
| | TLVTVSS | (SEQ ID |
| | (SEQ ID | NO: 53) |
| | NO: 52) | |
| TY23555 | EVQLVESGGGLVQPG | DIQLTQSPSSLSASV |
| | GSLRLSCAASGYSIS | GDRVTITCRASQSIS |
| | SGHYWAWIRQAPGKG | RVVLAWYQQKPGKAP |
| | LEWVSVISGSGGSTY | KLLIYAASSLQSGVP |
| | YADSVKGRFTISRDN | SRFSGSGSGTDFTLT |
| | SKNTLYLQLNSLRAE | ISSLQPEDFATYYCQ |
| | DTAVYYCARYRSGAF | QAYSIWTFGQGTKVE |
| | DYWGQGTLVTVSS | IKR |
| | (SEQ ID | (SEQ ID |
| | NO: 54) | NO: 55) |
| TY23556 | EVQLVESGGGLVQPG | DIQLTQSPSSLSASV |
| | GSLRLSCAASGYSIT | GDRVTITCSASSSVG |
| | SGHYWGWIRQAPGKG | YVQWYQQKPGKAPKL |
| | LEWIGEIYHSGSTYY | LIYDASNRATGIPSR |
| | SPSLKSRVTISRDNS | FSGSGSGTDFTLTIS |
| | KNTLYLQLNSLRAED | SLQPEDFATYYCQQY |
| | TAVYYCTRGRYGLDY | GSAPITFGQGTKVEI |
| | WGQGTLVTVSS | KR |
| | (SEQ ID | (SEQ ID |
| | NO: 56) | NO: 57) |
| TY23557 | EVQLVESGGGLVQPG | DIQLTQSPSSLSASV |
| | GSLRLSCAASGFTFT | GDRVTITCRASESIT |
| | GYGIHWVRQAPGKGL | SSYLAWYQQKPGKAP |
| | EWVSAISGSGGSTYY | KLLIYDASSLESGVP |
| | ADSVKGRFTISRDNS | SRFSGSGSGTDFTLT |
| | KNTLYLQLNSLRAED | ISSLQPEDFATYYCF |
| | TAVYYCARGAYYGGS | QGSHYPPTFGQGTKV |
| | YYFDYWGQGTLVTVS | EIKR |
| | S | (SEQ ID |
| | (SEQ ID | NO: 59) |
| | NO: 58) | |
| TY23558 | EVQLVESGGGLVQPG | DIQLTQSPSSLSASV |
| | GSLRLSCAASGYSIS | GDRVTITCRARSTVS |
| | SGHYWSWIRQAPGKG | FPYLHWYQQKPGKAP |
| | LEWIGEIYHSGSTYY | KLLIYDASNLETGVP |
| | SPSLKSRVTISRDNS | SRFSGSGSGTDFTLT |
| | KNTLYLQLNSLRAED | ISSLQPEDFATYYCQ |
| | TAVYYCAREGSDSYG | QSLEDPFTFGQGTKV |
| | GPDPFDIWGQGTLVT | EIKR |
| | VSS | (SEQ ID |
| | (SEQ ID | NO: 61) |
| | NO: 60) | |
| TY23559 | EVQLVESGGGLVQPG | DIQLTQSPSSLSASV |
| | GSLRLSCAASGYSIS | GDRVTITCSASSSVS |
| | SGHYWAWIRQAPGKG | YVQWYQQKPGKAPKL |
| | LEWIGEIYHSGSTYY | LIYDASNRATGIPSR |
| | SPSLKSRVTISRDNS | FSGSGSGTDFTLTIS |
| | KNTLYLQLNSLRAED | SLQPEDFATYYCQQY |
| | TAVYYCTRGRYGLDY | STNPLTFGQGTKVEI |
| | VVGQGTLVTVSS | KR |
| | (SEQ ID | (SEQ ID |
| | NO: 62) | NO: 63) |
| TY23561 | EVQLVESGGGLVQPG | DIQLTQSPSSLSASV |
| | GSLRLSCAASGFSLS | GDRVTITCRASQSVR |
| | TSGVGVSWIRQAPGK | GSYLAWYQQKPGKAP |
| | GLEWLALIDWAGDKY | KLLIYAASTLQSGVP |
| | YSPSLKSRLTISRDN | SRFSGSGSGTDFTLT |
| | SKNTLYLQLNSLRAE | ISSLQPEDFATYYCQ |
| | DTAVYYCARYGYSSY | QYSSLWTFGQGTKVE |
| | ALDYWGQGTLVTVSS | IKR |
| | | (SEQ ID |
| | (SEQ ID | NO: 65) |
| | NO: 64) | |
| TY23562 | EVQLVESGGGLVQPG | DIQLTQSPSSLSASV |
| | GSLRLSCAASGYSIS | GDRVTITCRASQSVD |
| | SGYHWDWIRQAPGKG | FVGKSFLDWYQQKPG |
| | LEWIGRINPNRGSTK | KAPKLLIYDASNLET |
| | YAQKFQGRVTISRDN | GVPSRFSGSGSGTDF |
| | SKNTLYLQLNSLRAE | TLTISSLQPEDFATY |
| | DTAVYYCAREFGAYW | YCQQRASWPLTFGQG |
| | GQGTLVTVSS | TKVEIKR |
| | (SEQ ID | (SEQ ID |
| | NO: 66) | NO: 67) |

TABLE 4
VH and VL sequences of affinity-
matured anti-CD137L Fab fragments

| Fab | VH | VL |
|---|---|---|
| Name | sequence | sequence |
| Fab8949 | EVQLVESGGG | DIQLTQSPSS |
| | LVQPGGSLRL | LSASVGDRVT |
| | SCAASGFSLN | ITCRASQSVR |
| | TAGVGVTWIR | GSYLAWYQQK |
| | QAPGKGLEWI | PGKAPKLLIY |
| | GLIDWAGYTY | AASTLQSGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSLWTFGQ |
| | GYGGYAIDYW | GTKVEIKR |
| | GQGTLVTVSS | (SEQ ID |
| | (SEQ ID | NO: 242) |
| | NO: 241) | |
| Fab8950 | EVQLVESGGG | DIQLTQSPSS |
| | LVQPGGSLRL | LSASVGDRVT |
| | SCAASGFSLS | ITCRASQSVR |
| | TSGVGVAWTR | GSYLAWYQQK |
| | QAPGKGLEWL | PGKAPKLLIY |
| | AVIDWAGYKY | AASTLQSGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYGSLWTFGQ |
| | GYRNYALDYW | GTKVEIKR |
| | GQGTLVTVSS | (SEQ ID |
| | (SEQ ID | NO: 244) |
| | NO: 243) | |
| Fab8954 | EVQLVESGGG | DIQLTQSPSS |
| | LVQPGGSLRL | LSASVGDRVT |
| | SCAASGFSLS | ITCRASQSVR |
| | TAGVGVSWIR | GNYLAWYQQK |
| | QAPGKGLEWI | PGKAPKLLIY |
| | AVIDWAGAKY | AASTLQSGVP |
| | YNPSLKSRLT | SRFSGSGSGT |
| | ISRDNSKNTL | DFTLTISSLQ |
| | YLQLNSLRAE | PEDFATYYCQ |
| | DTAVYYCARY | QYGSLWTFGQ |
| | GYRNYALDYW | GTKVEIKR |
| | GQGTLVTVSS | (SEQ ID |
| | (SEQ ID | NO: 246) |
| | NO: 245) | |
| Fab8959 | EVQLVESGGG | DIQLTQSPSS |
| | LVQPGGSLRL | LSASVGDRVT |
| | SCAASGFSLS | ITCRASQSVR |
| | TSGVGVAWIR | GSYLAWYQQK |
| | QAPGKGLEWL | PGKAPKLLIY |
| | AVIDWAGYKY | AASTLQSGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYGSYWTFGQ |
| | GYRNYALDYW | GTKVEIKR |
| | GQGTLVTVSS | (SEQ ID |
| | (SEQ ID | NO: 248) |
| | NO: 247) | |
| Fab8963 | EVQLVESGGG | DIQLTQSPSS |
| | LVQPGGSLRL | LSASVGDRVT |
| | SCAASGFSLS | ITCRASQSVR |
| | TSGVGVGWTR | GNYLAWYQQK |
| | QAPGKGLEWI | PGKAPKLLIY |
| | ALIDYAGSKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSYWTFOQ |
| | GYRSYAMDYW | GTKVEIKR |
| | GQGTLVTVSS | (SEQ ID |
| | (SEQ ID | NO: 250) |
| | NO: 249) | |
| Fab8964 | EVQLVESGGG | DIQLTQSPSS |
| | LVQPGGSLRL | LSASVGDRVT |
| | SCAASGFSLS | ITCRASQSVR |
| | TGGVGVAWIR | GSYLAWYQQK |
| | QAPGKGLEWL | PGKAPKLLIY |
| | GLIDWAGYTY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSLWTFGQ |
| | GYGSYALDYW | GTKVEIKR |
| | GQGTLVTVSS | (SEQ ID |
| | (SEQ ID | NO: 252) |
| | NO: 251) | |
| Fab8966 | EVQLVESGGG | DIQLTQSPSS |
| | LVQPGGSLRL | LSASVGDRVT |
| | SCAASGFSLS | ITCRASQSVS |
| | TGGVGVAWIR | GSYLAWYQQK |
| | QAPGKGLEWI | PGKAPKLLIY |
| | GLIDWAGYTY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYNSSWTFGQ |
| | GYRSYAMDYW | GTKVEIKR |
| | GQGTLVTVSS | (SEQ ID |
| | (SEQ ID | NO: 254) |
| | NO: 253) | |
| Fab8969 | EVQLVESGGG | DIQLTQSPSS |
| | LVQPGGSLRL | LSASVGDRVT |
| | SCAASGFSLS | ITCRASQTIR |
| | TAGVGVTWTR | GSYLAWYQQK |
| | QAPGKGLEWL | PGKAPKLLIY |
| | ALIDWAGDKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSLWTFGQ |
| | GVGSYALDYW | GTKVEIKR |
| | GQGTLVTVSS | (SEQ ID |
| | (SEQ ID | NO: 256) |
| | NO: 255) | |
| Fab8971 | EVQLVESGGG | DIQLTQSPSS |
| | LVQPGGSLRL | LSASVGDRVT |
| | SCAASGFSLS | ITCRASQSVR |
| | TAGVGVAWIR | GSYLAWYQQK |
| | QAPGKGLEWL | PGKAPKLLIY |
| | ALIDWAGDKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSYWTFOQ |
| | GYSSYALDYW | GTKVEIKR |
| | GQGTLVTVSS | (SEQ ID |
| | (SEQ ID | NO: 258) |
| | NO: 257) | |
| Fab8974 | EVQLVESGGG | DIQLTQSPSS |
| | LVQPGGSLRL | LSASVGDRVT |
| | SCAASGFSLT | ITCRASQSVR |
| | TTGVGVGWIR | GNYLAWYQQK |
| | QAPGKGLEWI | PGKAPKLLIY |
| | GLIDYAGYTY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSYWTFGQ |
| | GYDSYAMDYW | GTKVEIKR |
| | GQGTLVTVSS | (SEQ ID |
| | (SEQ ID | NO: 260) |
| | NO: 259) | |
| Fab8976 | EVQLVESGGG | DIQLTQSPSS |
| | LVQPGGSLRL | LSASVGDRVT |
| | SCAASGFSLT | ITCRASQSVR |
| | TSGVGVSWIR | NSYLAWYQQK |
| | QAPGKGLEWL | PGKAPKLLIY |
| | GLIDWSGYTY | DASSLESGVP |
| | YNPSLKSRLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYRNYALDYW | GTKVElKR |
| | GQGTLVTVSS | (SEQ ID |
| | (SEQ ID | NO: 262) |
| | NO: 261) | |
| Fab8985 | EVQLVESGGG | DIQLTQSPSS |
| | LVQPGGSLRL | LSASVGDRVT |
| | SCAASGFSLS | ITCRASQTVR |
| | TGGVGVGWIR | GTFLAWYQQK |
| | QAPGKGLEWL | PGKAPKLLIY |
| | ALIDWAGDKY | DASSLESGVP |
| | YSPSLKSRLT | YRFSGSGSGT |
| | ISRDNSKNTL | DFTLTISSLQ |
| | YLQLNSLRAE | PEDFATYYCQ |
| | DTAVYYCARY | QYSSLWTFGQ |
| | GWSSYALDYW | GTKVEIKR |
| | GQGTLVTVSS | (SEQ ID |
| | (SEQ ID | NO: 264) |
| | NO: 263) | |
| Fab8986 | EVQLVESGGG | DIQLTQSPSS |
| | LVQPGGSLRL | LSASVGDRVT |
| | SCAASGFSLS | ITCRASQSVR |
| | TGGVGVSWIR | GSYLAWYQQK |
| | QAPGKGLEWI | PGKAPKLLIY |
| | AVIDYAGYTY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYNSLWTFGQ |
| | GYNSYALDYW | GTKVEIKR |
| | GQGTLVTVSS | (SEQ ID |
| | (SEQ ID | NO: 266) |
| | NO: 265) | |
| Fab8996 | EVQLVESGGG | DIQLTQSPSS |
| | LVQPGGSLRL | LSASVGDRVT |
| | SCAASGFSLS | ITCRASQTVR |
| | TGGVGVTWIR | GTFLAWYQQK |
| | QAPGKGLEWL | PGKAPKLLIY |
| | ALIDWAGDTY | DASSLESGVP |
| | YSPSLKSRLT | YRFSGSGSGT |
| | ISRDNSKNTL | DFTLTISSLQ |
| | YLQLNSLRAE | PEDFATYYCQ |
| | DTAVYYCARY | QYSSLWTFGQ |
| | GVSSYALDYW | GTKVEIKR |
| | GQGTLVTVSS | (SEQ ID |
| | (SEQ ID | NO: 268) |
| | NO: 267) | |
| Fab9010 | EVQLVESGGG | DIQLTQSPSS |
| | LVQPGGSLRL | LSASVGDRVT |
| | SCAASGFSLT | ITCRASQSVR |
| | TGGVGVTWIR | GSYLAWYQQK |
| | QAPGKGLEWL | PGKAPKLLIY |
| | GLIDWSGDKY | DASSLESGVP |
| | YNPSLKSRLT | SRFSGSGSGT |
| | ISRDNSKNTL | DFTLTISSLQ |
| | YLQLNSLRAE | PEDFATYYCQ |
| | DTAVYYCARY | QYSSLWTFGQ |
| | GYDSYAMDYW | GTKVEIKR |
| | GQGTLVTVSS | (SEQ ID |
| | (SEQ ID | NO: 270) |
| | NO: 269) | |
| Fab9011 | EVQLVESGGG | DIQLTQSPSS |
| | LVQPGGSLRL | LSASVGDRVT |
| | SCAASGFSLT | ITCRASQSVR |
| | TAGVGVAYVI | GNYLAWYQQK |
| | RQAPGKGLEW | PGKAPKLLIY |
| | IGLIDWAGYK | DASSLESGVP |
| | YYSPSLKSRL | SRFSGSGSGT |
| | TISRDNSKNT | DFTLTISSLQ |
| | LYLQLNSLRA | PEDFATYYCQ |
| | EDTAVYYCAR | QYSSSWTFGQ |
| | YGYDGYAMDY | GTKVEIKR |
| | WCiQGTLVTVSS | (SEQ ID |
| | (SEQ ID | NO: 272) |
| | NO: 271) | |
| Fab9012 | EVQLVESGGG | DIQLTQSPSS |
| | LVQPGGSLRL | LSASVGDRVT |
| | SCAASGFSLS | ITCRASQSVR |
| | TAGVGVTWIR | GTFLAWYQQK |
| | QAPGKGLEWL | PGKAPKLLIY |
| | ALIDWAGDKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSWWTFGQ |
| | GYSSYALDYW | GTKVEIKR |
| | GQGTLVTVSS | (SEQ ID |
| | (SEQ ID | NO: 274) |
| | NO: 273) | |
| Fab9013 | EVQLVESGGG | DIQLTQSPSS |
| | LVQPGGSLRL | LSASVGDRVT |
| | SCAASGFSLN | ITCRASQSVR |
| | TTGVGVSWIR | GSYLAWYQQK |
| | QAPGKGLEWI | PGKAPKLLIY |
| | GLIDWAGYTY | DASSLESGVP |
| | YNPSLKSRLT | SRFSGSGSGT |
| | ISRDNSKNTL | DFTLTISSLQ |
| | YLQLNSLRAE | PEDFATYYCQ |
| | DTAVYYCARY | QYSSLWTFGQ |
| | GYSGYALDYW | GTKVEIKR |
| | GQGTLVTVSS | (SEQ ID |
| | (SEQ ID | NO: 276) |
| | NO: 275) | |
| Fab9014 | EVQLVESGGG | DIQLTQSPSS |
| | LVQPGGSLRL | LSASVGDRVT |
| | SCAASGFSLS | ITCRASQSVR |
| | TAGVGVAWIR | GSYLAWYQQK |
| | QAPGKGLEWI | PGKAPKLLIY |
| | GLDWAGSKYY | DASSLESGVP |
| | NPSLKSRLTI | SRFSGSGSGT |
| | SRONSKNTLY | DFTLTISSLQ |
| | LQLNSLRAED | PEDFATYYCQ |
| | TAVYYCARYG | QYSSSWTFGQ |
| | YSGYAMDYWG | GTKVEIKR |
| | QGTLVTVSS | (SEQ ID |
| | (SEQ ID | NO: 278) |
| | NO: 277) | |
| Fab9015 | EVQLVESGGG | DIQLTQSPSS |
| | LVQPGGSLRL | LSASVGDRVT |
| | SCAASGFSLT | ITCRASOSVR |
| | TGGVGVAWIR | GSYLAWYQQK |
| | QAPGKGLEWL | PGKAPKLLIY |
| | AVIDWAGYKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSFWTFGQ |
| | GYNSYALDYW | GTKVEIKR |
| | GQGTLVTVSS | (SEQ ID |
| | (SEQ ID | NO: 280) |
| | NO: 279) | |
| Fab9016 | EVQLVESGGG | DIQLTQSPSS |
| | LVQPGGSLRL | LSASVGDRVT |
| | SCAASGFSLS | ITCRASQTVR |
| | TGGVGVAWIR | GTFLAWYQQK |
| | QAPGKGLEWL | PGKAPKLLIY |
| | ALIDWAGDKY | DASSLESGVP |
| | YSPSLKSRLT | YRFSGSGSGT |
| | ISRDNSKNTL | DFTLTISSLQ |
| | YLQLNSLRAE | PEDFATYYCQ |
| | DTAVYYCARY | QYSSLWTFGQ |
| | GYGSYALDYW | GTKVEIKR |
| | GQGTLVTVSS | (SEQ ID |
| | (SEQ ID | NO: 282) |
| | NO: 281) | |
| Fab9017 | EVQLVESGGG | DIQLTQSPSS |
| | LVQPGGSLRL | LYASVGDRVT |
| | SCAASGFSLS | ITCRASQTVR |
| | TGGVGVGWIR | GSYLAWYQQK |
| | QAPGKGLEWL | PGKAPKLLIY |
| | ALIDWAGDKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSLWTFGQ |
| | GYGSYALDYW | GTKVEIKR |
| | GQGTLVTVSS | (SEQ ID |
| | (SEQ ID | NO: 284) |
| | NO: 283) | |
| Fab90I8 | EVQLVESGGG | DIQLTQSPSS |
| | LVQPGGSLRL | LSASVGDRVT |
| | SCAASGFSLN | ITCRASQSVR |
| | TGGVGVTWIR | GNYLAWYQQK |
| | QAPGKGLEWI | PGKAPKLLIY |
| | GLIDWAGYKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYDSYALDYW | GTKVEIKR |
| | GQGTLVTVSS | (SEQ ID |
| | (SEQ ID | NO: 286) |
| | NO: 285) | |
| Fab9019 | EVQLVESGGG | DIQLTQSPSS |
| | LVQPGGSLRL | LSASVGDRVT |
| | SCAASGFSLS | ITCRASQTVR |
| | TAGVGVAWIR | GNYLAWYQQK |
| | QAPGKGLEWL | PGKAPKLLIY |
| | ALIDWADDKY | AASTLQSGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYGSLWTFGQ |
| | GYSNYALDYW | GTKVEJKR |
| | GQGTLVTVSS | (SEQ ID |
| | (SEQ ID | NO: 288) |
| | NO: 287) | |
| Fab9020 | EVQLVESGGG | DIQLTQSPSS |
| | LVQPGGSLRL | LSASVGDRVT |
| | SCAASGFSLS | ITCRASQTVR |
| | TAGVGVAWIR | GTFLAWYQQK |
| | QAPGKGLEWL | PGKAPKLLIY |
| | ALIDWAGDTY | DASSLESGVP |
| | YSPSLKSRLT | YRFSGSGSGT |
| | ISRDNSKNTL | DFTLTISSLQ |
| | YLQLNSLRAE | PEDFATYYCQ |
| | DTAVYYCARY | QYSSLWTFGQ |
| | GLSSYALDYW | GTKVEIKR |
| | GQGTLVTVSS | (SEQ ID |
| | (SEQ ID | NO: 290) |
| | NO: 289) | |
| Fab9022 | EVQLVESGGG | DIQLTQSPSS |
| | LVQPGGSLRL | LSASVGDRVT |
| | SCAASGFSLS | ITCRASQSVR |
| | TAGVGVAWIR | GNYLAWYQQK |
| | QAPGKGLEWL | PGKAPKLLIY |
| | ALIDYAGDKY | DASSLESGVP |
| | YNPSPKSHLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYSGYALDYW | GTKVEIKR |
| | GQGTLVTVSS | (SEQ ID |
| | (SEQ ID | NO: 292) |
| | NO: 29I) | |
| Fab9023 | EVQLVESGGG | DIQLTQSPSS |
| | LVQPGGSLRL | LSASVGDRVT |
| | SCAASGFSLS | ITCRASQSVR |
| | TGGVGVGWIR | GSYLAWYQQK |
| | QAPGKGLEWL | PGKAPKLLIY |
| | AVIDYAGYTY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYGSYALDYW | GTKVEIKR |
| | GQGTLVTVSS | (SEQ ID |
| | (SEQ ID | NO: 294) |
| | NO: 293) | |
| Fab9024 | EVQLVESGGG | DIQLTQSPSS |
| | LVQPGGSLRL | LSASVGDRVT |
| | SCAASGFSLS | ITCRASQSVR |
| | TAGVGVGWIR | GSYLAWYQQK |
| | QAPGKGLEWL | PGKAPKLLIY |
| | ALIDYAGYTY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSYWTFGQ |
| | GYSSYALDYW | CTKVEIKR |
| | GQGTLVTVSS | (SEQ ID |
| | (SEQ ID | NO: 296) |
| | NO: 295) | |
| Fab9025 | EVQLVESGGG | DIQLTQSPSS |
| | LVQPGGSLRL | LSASVGDRVT |
| | SCAASGFSLS | ITCRASQSVR |
| | TSGVGVAWIR | GSYLAWYQQK |
| | QAPGKGLEWL | PGKAPKLLIY |
| | ALIDYAGYTY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYSSYALDYW | GTKVElKR |
| | GQGTLVTVSS | (SEQ ID |
| | (SEQ ID | NO: 298) |
| | NO: 297) | |
| Fab9026 | EVQLVESGGG | DIQLTQSPSS |
| | LVQPGGSLRL | LSASVGDRVT |
| | SCAASGFSLS | ITCRASQSIR |
| | TGGVGVSWTR | GNYLAWYQQK |
| | QAPGKGLEWL | PGKAPKLLIY |
| | ALIDYAGYTY | AAFTLQSGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYSGYALDYW | GTKVEIKR |
| | GQGTLVTVSS | (SEQ ID |
| | (SEQ ID | NO: 300) |
| | NO: 299) | |
| Fab9027 | EVQLVESGGG | DIQLTQSPSS |
| | LVQPGGSLRL | LSASVGDRVT |
| | SCAASGFSLS | ITCRTSQSVR |
| | TSGVGVTWIR | GSYLAWYQQK |
| | QAPGKGLEWL | PGKAPKLLIY |
| | ALIDYAGYKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYGSYALDYW | GTKVEIKR |
| | GQGTLVTVSS | (SEQ ID |
| | (SEQ ID | NO: 302) |
| | NO: 301) | |
| Fab9028 | EVQLVESGGG | DIQLTQSPSS |
| | LVQPGGSLRL | LSASVGDRVT |
| | SCAASGFSLS | ITCRASQSIR |
| | TGGVGVAWIR | GSYLAWYQQK |
| | QAPGKGLEWL | PGKAPKLLIY |
| | ALIDWSGDKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYGSYALDYW | GTKVEIKR |
| | GQGTLVTVSS | (SEQ ID |
| | (SEQ ID | NO: 304) |
| | NO: 303) | |
| Fab9029 | EVQLVESGGG | DIQLTQSPSS |
| | LVQPGGSLRL | LSASVGDRVT |
| | SCAASGFSLS | ITCRASQSIR |
| | TSGVGVAWIR | GSYLAWYQQK |
| | QAPGKGLEWL | PGKAPKLLIY |
| | ALIDYSGATY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYGSYALDYW | GTKVEIKR |
| | GQGTLVTVSS | (SEQ ID |
| | (SEQ ID | NO: 306) |
| | NO: 305) | |
| Fab9030 | EVQLVESGGG | DIQLTQSPSS |
| | LVQPGGSLRL | LSASVGDRVT |
| | SCAASGFSLS | ITCRASQSIH |
| | TSGVGVAWIR | GSYLAWYQQK |
| | QAPGKGLEWL | PGKAPKLLIY |
| | ALIDYAGYTY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYSGYALDYW | GTKVEIKR |
| | GQGTLVTVSS | (SEQ ID |
| | (SEQ ID | NO: 308) |
| | NO: 307) | |
| Fab9031 | EVQLVESGGG | DIQLTQSPSS |
| | LVQPGGSLRL | LSASVGDRVT |
| | SCAASGFSLS | ITCRASQSVR |
| | TGGVGVAWIR | GSYLAWYQQK |
| | QAPGKGLEWL | PGKAPKLLIY |
| | ALIDYSGDKY | DASSLESGVP |
| | YSPSPKSRLT | SRFSGSGSGT |
| | ISRDNSKNTL | DFTLTISSLQ |
| | YLQLNSLRAE | PEDFATYYCQ |
| | DTAVYYCARY | QYSSFWTFGQ |
| | GYGSYALDYW | GTKVEIKR |
| | GQGTLVTVSS | (SEQ ID |
| | (SEQ ID | NO: 310) |
| | NO: 309) | |
| Fab9032 | EVQLVESGGG | YIQLTQSPSS |
| | LVQPGGSLRL | LSASVGDRVT |
| | SCAASGFSLS | ITCRASQSIR |
| | TAGVGVSWIR | GSYLAWYQQK |
| | QAPGKGLEWL | PGKAPKLLIY |
| | ALIDYAGSKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSLWTFGQ |
| | GYGSYALDYW | GTKVEIKR |
| | GQGTLVTVSS | (SEQ ID |
| | (SEQ ID | NO: 312) |
| | NO: 311) | |
| Fab9033 | EVQLVESGGG | DIQLTQSPSS |
| | LVQPGGSLRL | LSASVGDRVT |
| | SCAASGFSLS | ITCRASQSIR |
| | TGGVGVAWIR | GSYLAWYQQK |
| | QAPGKGLEWL | PGKAPKLLIY |
| | ALIDWAGDKY | DASSLESGVP |
| | YNPSLKSRLT | SRFSGSGSGT |
| | ISRDNSKNTL | DFTLTISSLQ |
| | YLQLNSLRAE | PEDFATYYCQ |
| | DTAVYYCARY | QYSSFWTFGQ |
| | GYGSYALDYW | GTKVEIKR |
| | GQGTLVTVSS | (SEQ ID |
| | (SEQIDNO;313) | NO: 314) |
| Fab9036 | EVQLVESGGG | DIQLTQSPSS |
| | LVQPGGSLRL | LSASVGDRVT |
| | SCAASGFSLS | ITCRASQSVR |
| | TGGVGVAWIR | GNYLAWYQQK |
| | QAPGKGLEWL | PGKAPKLLIY |
| | ALIDYAGYKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSFWTFGQ |
| | GYGGYALDYW | GTKVEIKR |
| | GQGTLVTVSS | (SEQ ID |
| | (SEQ ID | NO: 316) |
| | NO: 315) | |
| Fab9037 | EVQLVESGGG | DIQLTQSPSS |
| | LVQPGGSLRL | LSASVGDRVT |
| | SCAASGFSLS | ITCRASQSVH |
| | TTGVGVAW | GSYLAWYQQK |
| | IRQAPGK | PGKAPKLLIY |
| | GLEWLAIIDW | DASSLESGVP |
| | SGSKYYSPSL | SRFSGSGSGT |
| | KSRLTISRDN | DFTLTISSLQ |
| | SKNTLYLQLN | PEDFATYYCQ |
| | SLRAEDTAVY | QYNSLWTFGQ |
| | YCARYGYSSY | GTKVEIKR |
| | ALDYWGQGTL | (SEQ ID |
| | VTVSS | NO: 318) |
| | (SEQ ID | |
| | NO: 317) | |
| Fab9038 | EVQLVESGGG | DIQLTQSPSS |
| | LVQPGGSLRL | LSASVGDRVT |
| | SCAASGFSLS | ITCRASQGVS |
| | TAGVGVGWIR | SYLAWYQQKP |
| | QAPGKGLEWL | GKAPKLLIYD |
| | ALIDWSGATY | ASSLESGVPS |
| | YSPSLKSRLT | RFSGSGSGTD |
| | ISRDNSKNTL | FTLTISSLQP |
| | YLQLNSLRAE | EDFATYYCQQ |
| | DTAVYYCARY | YGSYWTFGQG |
| | GYSGYALDYW | TKVEIKR |
| | GQGTLVTVSS | (SEQ ID |
| | (SEQ ID | NO: 320) |
| | NO: 319) | |
| Fab9039 | EVQLVESGGG | DIQLTQSPSS |
| | LVQPGGSLRL | LSASVGDRVT |
| | SCAASGFSLS | ITCRASQSIR |
| | TAGVGVAWIR | GNYLAWYQQK |
| | QAPGKGLEWL | PGKAPKLLIY |
| | ALIDYAGDKY | DASSLESGVP |
| | YNPSPKSHLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYSGYALDYW | GTKVEIKR |
| | GQGTLVTVSS | (SEQ ID |
| | (SEQ ID | NO: 322) |
| | NO: 321) | |
| Fab8955 | EVQLVESGGG | DIQLTQSPSS |
| | LVQPGGSLRL | LSASVGDRVT |
| | SCAASGFSLS | ITCRASQTVR |
| | TGGVGVSWIR | GSYLAWYQQK |
| | QAPGKGLEWL | PGKAPKLLIY |
| | ALIDWAGDKY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSLWTFGQ |
| | GYGSYALDYW | GTKVEIKR |
| | GQGTLVTVSS | (SEQ ID |
| | (SEQ ID | NO: 324) |
| | NO: 323) | |
| Fab8957 | EVQLVESGGG | DIQLTQSPSS |
| | LVQPGGSLRL | LSASVGDRVT |
| | SCAASGFSLS | ITCRASQTVR |
| | TAGVGVSWTR | GTYLAWYQQK |
| | QAPGKGLEWL | PGKAPKLLIY |
| | ALIDWAGDTY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSLWTFGQ |
| | GYSSYALDYW | GTKVEIKR |
| | GQGTLVTVSS | (SEQ ID |
| | (SEQ ID | NO: 326) |
| | NO: 325) | |
| Fab8958 | EVQLVESGGG | DIQLTQSPSS |
| | LVQPGGSLRL | LSASVGDRVT |
| | SCAASGFSLS | ITCRASQTVR |
| | TGGVGVAWIR | GTFLAWYQQK |
| | QAPGKGLEWL | PGKAPKLLIY |
| | ALIDWAGDKY | DASSLESGVP |
| | YSPSLKSRLT | YRFSGSGSGT |
| | ISRDNSKNTL | DFTLTISSLQ |
| | YLQLNSLRAE | PEDFATYYCQ |
| | DTAVYYCARY | QYSSLWTFGQ |
| | GGSSYALDYW | GTKVEIKR |
| | GQGTLVTVSS | (SEQ ID |
| | (SEQ ID | NO: 328) |
| | NO: 327) | |
| Fab8947 | EVQLVESGGG | DIQLTQSPSS |
| | LVQPGGSLRL | LSASVGDRVT |
| | SCAASGFSLN | ITCRASQSVR |
| | TAGVGVSWIR | GNYLAWYQQK |
| | QAPGKGLEWL | PGKAPKLLIY |
| | ALIDWAGYKY | AASSLQSGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSYWTFGQ |
| | GYRNYAMDYW | GTKVETKR |
| | GQGTLVTVSS | (SEQ ID |
| | (SEQ ID | NO: 330) |
| | NO: 329) | |
| Fab8948 | EVQLVESGGG | DIQLTQSPSS |
| | LVQPGGSLRL | LSASVGDRVT |
| | SCAASGFSLT | ITCRASQSVR |
| | TSGVGVAWIR | GSYLAWYQQK |
| | QAPGKGLEWI | PGKAPKLLIY |
| | GLIDHAGYTY | DASSLESGVP |
| | YSPSLKSRLT | SRFSGSGSGT |
| | ISRDNSKNTL | DFTLTISSLQ |
| | YLQLNSLRAE | PEDFATYYCQ |
| | DTAVYYCARY | QYSSSWTFGQ |
| | GYDSYAMDYW | GTKVEIKR |
| | GQGTLVTVSS | (SEQ ID |
| | (SEQ ID | NO: 332) |
| | NO: 331) | |
| Fab8951 | EVQLVESGGG | DIQLTQSPSS |
| | LVQPGGSLRL | LSASVGDRVT |
| | SCAASGFSLT | ITCRASQSVR |
| | TSGVGVGWIR | GNYLAWYQQK |
| | QAPGKGLEW | |
| | IGLIDYAGYK | PGKAPKLLIY |
| | YYSPSLKSRL | DASSLESGVP |
| | TISRDNSKNT | SRFSGSGSGT |
| | LYLQLNSLRA | DFTLTISSLQ |
| | EDTAVYYCAR | PEDFATYYCQ |
| | YGYRGYALDY | QYSSFWTFGQ |
| | WGQGTLVTVS | GTKVEIKR |
| | S | (SEQ ID |
| | (SEQ ID | NO: 334) |
| | NO: 333) | |
| Fab8952 | EVQLVESGGG | DIQLTQSPSS |
| | LVQPGGSLRL | LSASVGDRVT |
| | SCAASGFSLS | ITCRASQTVR |
| | TAGVGVAWIR | GTFLAWYQQK |
| | QAPGKGLEWL | PGKAPKLLIY |
| | ALIDWAGDTY | DASSLESGVP |
| | YSPSLKSRLT | YRFSGSGSGT |
| | ISRDNSKNTL | DFTLTISSLQ |
| | YLQLNSLRAE | PEDFATYYCQ |
| | DTAVYYCARY | QYSSLWTFGQ |
| | GVGSYALDYW | GTKVEIKR |
| | GQGTLVTVSS | (SEQ ID |
| | (SEQ ID | NO: 336) |
| | NO: 335) | |

In some embodiments, the anti-CD137L antibody comprises VH and/or VL region sequences of a single antibody listed in Table 3 or Table 4.

In some embodiments, the antibody comprises a heavy chain variable region and a light chain variable region, wherein the heavy chain variable region comprises the amino acid sequence of SEQ ID NO:52, and/or the light chain variable region comprises the amino acid sequence of SEQ ID NO:53. In some embodiments, the antibody comprises a heavy chain variable region and a light chain variable region, wherein the heavy chain variable region comprises the amino acid sequence of SEQ ID NO:54, and/or the light chain variable region comprises the amino acid sequence of SEQ ID NO:55. In some embodiments, the antibody comprises a heavy chain variable region and a light chain variable region, wherein the heavy chain variable region comprises the amino acid sequence of SEQ ID NO:56, and/or the light chain variable region comprises the amino acid sequence of SEQ ID NO:57. In some embodiments, the antibody comprises a heavy chain variable region and a light chain variable region, wherein the heavy chain variable region comprises the amino acid sequence of SEQ ID NO:58, and/or the light chain variable region comprises the amino acid sequence of SEQ ID NO:59. In some embodiments, the antibody comprises a heavy chain variable region and a light chain variable region, wherein the heavy chain variable region comprises the amino acid sequence of SEQ ID NO:60, and/or the light chain variable region comprises the amino acid sequence of SEQ ID NO:61. In some embodiments, the antibody comprises a heavy chain variable region and a light chain variable region, wherein the heavy chain variable region comprises the amino acid sequence of SEQ ID NO:62, and/or the light chain variable region comprises the amino acid sequence of SEQ ID NO:63. In some embodiments, the antibody comprises a heavy chain variable region and a light chain variable region, wherein the heavy chain variable region comprises the amino acid sequence of SEQ ID NO:64, and/or the light chain variable region comprises the amino acid sequence of SEQ ID NO:65. In some embodiments, the antibody comprises a heavy chain variable region and a light chain variable region, wherein the heavy chain variable region comprises the amino acid sequence of SEQ ID NO:66, and/or the light chain variable region comprises the amino acid sequence of SEQ ID NO:67. In some embodiments, the anti-CD137L antibody comprises 1, 2, or 3 HVR sequences from a VH region comprising the amino acid sequence of SEQ ID NO:52 and/or 1, 2, or 3 HVR sequences from a VL region comprising the amino acid sequence of SEQ ID NO:53. In some embodiments, the anti-CD137L antibody comprises 1, 2, or 3 HVR sequences from a VH region comprising the amino acid sequence of SEQ ID NO:54 and/or 1, 2, or 3 HVR sequences from a VL region comprising the amino acid sequence of SEQ ID NO:55. In some embodiments, the anti-CD137L antibody comprises 1, 2, or 3 HVR sequences from a VH region comprising the amino acid sequence of SEQ ID NO:56 and/or 1, 2, or 3 HVR sequences from a VL region comprising the amino acid sequence of SEQ ID NO:57. In some embodiments, the anti-CD137L antibody comprises 1, 2, or 3 HVR sequences from a VH region comprising the amino acid sequence of SEQ ID NO:58 and/or 1, 2, or 3 HVR sequences from a VL region comprising the amino acid sequence of SEQ ID NO:59. In some embodiments, the anti-CD137L antibody comprises 1, 2, or 3 HVR sequences from a VH region comprising the amino acid sequence of SEQ ID NO:60 and/or 1, 2, or 3 HVR sequences from a VL region comprising the amino acid sequence of SEQ ID NO:61. In some embodiments, the anti-CD137L antibody comprises 1, 2, or 3 HVR sequences from a VH region comprising the amino acid sequence of SEQ ID NO:62 and/or 1, 2, or 3 HVR sequences from a VL region comprising the amino acid sequence of SEQ ID NO:63. In some embodiments, the anti-CD137L antibody comprises 1, 2, or 3 HVR sequences from a VH region comprising the amino acid sequence of SEQ ID NO:64 and/or 1, 2, or 3 HVR sequences from a VL region comprising the amino acid sequence of SEQ ID NO:65. In some embodiments, the anti-CD137L antibody comprises 1, 2, or 3 HVR sequences from a VH region comprising the amino acid sequence of SEQ ID NO:66 and/or 1, 2, or 3 HVR sequences from a VL region comprising the amino acid sequence of SEQ ID NO:67.

In some embodiments, the anti-CD137L antibody comprises 1, 2, or 3 HVR sequences from a VH region comprising the amino acid sequence of SEQ ID NO:241 and/or 1, 2, or 3 HVR sequences from a VL region comprising the amino acid sequence of SEQ ID NO:242. In some embodiments, the anti-CD137L antibody comprises a heavy chain variable (VH) region comprising the amino acid sequence of SEQ ID NO:241 and/or a light chain variable (VL) region comprising the amino acid sequence of SEQ ID NO:242. In some embodiments, the anti-CD137L antibody comprises 1, 2, or 3 HVR sequences from a VH region comprising the amino acid sequence of SEQ ID NO:243 and/or 1, 2, or 3 HVR sequences from a VL region comprising the amino acid sequence of SEQ ID NO:244. In some embodiments, the anti-CD137L antibody comprises a heavy chain variable (VH) region comprising the amino acid sequence of SEQ ID NO:243 and/or a light chain variable (VL) region comprising the amino acid sequence of SEQ ID NO:244. In some embodiments, the anti-CD137L antibody comprises 1, 2, or 3 HVR sequences from a VH region comprising the amino acid sequence of SEQ ID NO:245 and/or 1, 2, or 3 HVR sequences from a VL region comprising the amino acid sequence of SEQ ID NO:246. In some embodiments, the anti-CD137L antibody comprises a heavy chain variable (VH) region comprising the amino acid sequence of SEQ ID NO:245 and/or a light chain variable (VL) region comprising the amino acid sequence of SEQ ID NO:246. In some embodiments, the anti-CD137L antibody comprises 1, 2, or 3 HVR sequences from a VH region comprising the amino acid sequence of SEQ ID NO:247 and/or 1, 2, or 3 HVR sequences from a VL region comprising the amino acid sequence of SEQ ID NO:248. In some embodiments, the anti-CD137L antibody comprises a heavy chain variable (VH) region comprising the amino acid sequence of SEQ ID NO:247 and/or a light chain variable (VL) region comprising the amino acid sequence of SEQ ID NO:248. In some embodiments, the anti-CD137L antibody comprises 1, 2, or 3 HVR sequences from a VH region comprising the amino acid sequence of SEQ ID NO:249 and/or 1, 2, or 3 HVR sequences from a VL region comprising the amino acid sequence of SEQ ID NO:250. In some embodiments, the anti-CD137L antibody comprises a heavy chain variable (VH) region comprising the amino acid sequence of SEQ ID NO:249 and/or a light chain variable (VL) region comprising the amino acid sequence of SEQ ID NO:250. In some embodiments, the anti-CD137L antibody comprises 1, 2, or 3 HVR sequences from a VH region comprising the amino acid sequence of SEQ ID NO:251 and/or 1, 2, or 3 HVR sequences from a VL region comprising the amino acid sequence of SEQ ID NO:252. In some embodiments, the anti-CD137L antibody comprises a heavy chain variable (VH) region comprising the amino acid sequence of SEQ ID NO:251 and/or a light chain variable (VL) region comprising the amino acid sequence of SEQ ID NO:252. In some embodiments, the anti-CD137L antibody comprises 1, 2, or 3 HVR sequences from a VH region comprising the amino acid sequence of SEQ ID NO:253 and/or 1, 2, or 3 HVR sequences from a VL region comprising the amino acid sequence of SEQ ID NO:254. In some embodiments, the anti-CD137L antibody comprises a heavy chain variable (VH) region comprising the amino acid sequence of SEQ ID NO:253 and/or a light chain variable (VL) region comprising the amino acid sequence of SEQ ID NO:254. In some embodiments, the anti-CD137L antibody comprises 1, 2, or 3 HVR sequences from a VH region comprising the amino acid sequence of SEQ ID NO:255 and/or 1, 2, or 3 HVR sequences from a VL region comprising the amino acid sequence of SEQ ID NO:256. In some embodiments, the anti-CD137L antibody comprises a heavy chain variable (VH) region comprising the amino acid sequence of SEQ ID NO:255 and/or a light chain variable (VL) region comprising the amino acid sequence of SEQ ID NO:256. In some embodiments, the anti-CD137L antibody comprises 1, 2, or 3 HVR sequences from a VH region comprising the amino acid sequence of SEQ ID NO:257 and/or 1, 2, or 3 HVR sequences from a VL region comprising the amino acid sequence of SEQ ID NO:258. In some embodiments, the anti-CD137L antibody comprises a heavy chain variable (VH) region comprising the amino acid sequence of SEQ ID NO:257 and/or a light chain variable (VL) region comprising the amino acid sequence of SEQ ID NO:258. In some embodiments, the anti-CD137L antibody comprises 1, 2, or 3 HVR sequences from a VH region comprising the amino acid sequence of SEQ ID NO:259 and/or 1, 2, or 3 HVR sequences from a VL region comprising the amino acid sequence of SEQ ID NO:260. In some embodiments, the anti-CD137L antibody comprises a heavy chain variable (VH) region comprising the amino acid sequence of SEQ ID NO:259 and/or a light chain variable (VL) region comprising the amino acid sequence of SEQ ID NO:260. In some embodiments, the anti-CD137L antibody comprises 1, 2, or 3 HVR sequences from a VH region comprising the amino acid sequence of SEQ ID NO:261 and/or 1, 2, or 3 HVR sequences from a VL region comprising the amino acid sequence of SEQ ID NO:262. In some embodiments, the anti-CD137L antibody comprises a heavy chain variable (VH) region comprising the amino acid sequence of SEQ ID NO:261 and/or a light chain variable (VL) region comprising the amino acid sequence of SEQ ID NO:262. In some embodiments, the anti-CD137L antibody comprises 1, 2, or 3 HVR sequences from a VH region comprising the amino acid sequence of SEQ ID NO:263 and/or 1, 2, or 3 HVR sequences from a VL region comprising the amino acid sequence of SEQ ID NO:264. In some embodiments, the anti-CD137L antibody comprises a heavy chain variable (VH) region comprising the amino acid sequence of SEQ ID NO:263 and/or a light chain variable (VL) region comprising the amino acid sequence of SEQ ID NO:264. In some embodiments, the anti-CD137L antibody comprises 1, 2, or 3 HVR sequences from a VH region comprising the amino acid sequence of SEQ ID NO:265 and/or 1, 2, or 3 HVR sequences from a VL region comprising the amino acid sequence of SEQ ID NO:266. In some embodiments, the anti-CD137L antibody comprises a heavy chain variable (VH) region comprising the amino acid sequence of SEQ ID NO:265 and/or a light chain variable (VL) region comprising the amino acid sequence of SEQ ID NO:266. In some embodiments, the anti-CD137L antibody comprises 1, 2, or 3 HVR sequences from a VH region comprising the amino acid sequence of SEQ ID NO:267 and/or 1, 2, or 3 HVR sequences from a VL region comprising the amino acid sequence of SEQ ID NO:268. In some embodiments, the anti-CD137L antibody comprises a heavy chain variable (VH) region comprising the amino acid sequence of SEQ ID NO:267 and/or a light chain variable (VL) region comprising the amino acid sequence of SEQ ID NO:268. In some embodiments, the anti-CD137L antibody comprises 1, 2, or 3 HVR sequences from a VH region comprising the amino acid sequence of SEQ ID NO:269 and/or 1, 2, or 3 HVR sequences from a VL region comprising the amino acid sequence of SEQ ID NO:270. In some embodiments, the anti-CD137L antibody comprises a heavy chain variable (VH) region comprising the amino acid sequence of SEQ ID NO:269 and/or a light chain variable (VL) region comprising the amino acid sequence of SEQ ID NO:270. In some embodiments, the anti-CD137L antibody comprises 1, 2, or 3 HVR sequences from a VH region comprising the amino acid sequence of SEQ ID NO:271 and/or 1, 2, or 3 HVR sequences from a VL region comprising the amino acid sequence of SEQ ID NO:272. In some embodiments, the anti-CD137L antibody comprises a heavy chain variable (VH) region comprising the amino acid sequence of SEQ ID NO:271 and/or a light chain variable (VL) region comprising the amino acid sequence of SEQ ID NO:272. In some embodiments, the anti-CD137L antibody comprises 1, 2, or 3 HVR sequences from a VH region comprising the amino acid sequence of SEQ ID NO:273 and/or 1, 2, or 3 HVR sequences from a VL region comprising the amino acid sequence of SEQ ID NO:274. In some embodiments, the anti-CD137L antibody comprises a heavy chain variable (VH) region comprising the amino acid sequence of SEQ ID NO:273 and/or a light chain variable (VL) region comprising the amino acid sequence of SEQ ID NO:274. In some embodiments, the anti-CD137L antibody comprises 1, 2, or 3 HVR sequences from a VH region comprising the amino acid sequence of SEQ ID NO:275 and/or 1, 2, or 3 HVR sequences from a VL region comprising the amino acid sequence of SEQ ID NO:276. In some embodiments, the anti-CD137L antibody comprises a heavy chain variable (VH) region comprising the amino acid sequence of SEQ ID NO:275 and/or a light chain variable (VL) region comprising the amino acid sequence of SEQ ID NO:276. In some embodiments, the anti-CD137L antibody comprises 1, 2, or 3 HVR sequences from a VH region comprising the amino acid sequence of SEQ ID NO:277 and/or 1, 2, or 3 HVR sequences from a VL region comprising the amino acid sequence of SEQ ID NO:278. In some embodiments, the anti-CD137L antibody comprises a heavy chain variable (VH) region comprising the amino acid sequence of SEQ ID NO:277 and/or a light chain variable (VL) region comprising the amino acid sequence of SEQ ID NO:278. In some embodiments, the anti-CD137L antibody comprises 1, 2, or 3 HVR sequences from a VH region comprising the amino acid sequence of SEQ ID NO:279 and/or 1, 2, or 3 HVR sequences from a VL region comprising the amino acid sequence of SEQ ID NO:280. In some embodiments, the anti-CD137L antibody comprises a heavy chain variable (VH) region comprising the amino acid sequence of SEQ ID NO:279 and/or a light chain variable (VL) region comprising the amino acid sequence of SEQ ID NO:280. In some embodiments, the anti-CD137L antibody comprises 1, 2, or 3 HVR sequences from a VH region comprising the amino acid sequence of SEQ ID NO:281 and/or 1, 2, or 3 HVR sequences from a VL region comprising the amino acid sequence of SEQ ID NO:282. In some embodiments, the anti-CD137L antibody comprises a heavy chain variable (VH) region comprising the amino acid sequence of SEQ ID NO:281 and/or a light chain variable (VL) region comprising the amino acid sequence of SEQ ID NO:282. In some embodiments, the anti-CD137L antibody comprises 1, 2, or 3 HVR sequences from a VH region comprising the amino acid sequence of SEQ ID NO:283 and/or 1, 2, or 3 HVR sequences from a VL region comprising the amino acid sequence of SEQ ID NO:284. In some embodiments, the anti-CD137L antibody comprises a heavy chain variable (VH) region comprising the amino acid sequence of SEQ ID NO:283 and/or a light chain variable (VL) region comprising the amino acid sequence of SEQ ID NO:284. In some embodiments, the anti-CD137L antibody comprises 1, 2, or 3 HVR sequences from a VH region comprising the amino acid sequence of SEQ ID NO:285 and/or 1, 2, or 3 HVR sequences from a VL region comprising the amino acid sequence of SEQ ID NO:286. In some embodiments, the anti-CD137L antibody comprises a heavy chain variable (VH) region comprising the amino acid sequence of SEQ ID NO:285 and/or a light chain variable (VL) region comprising the amino acid sequence of SEQ ID NO:286. In some embodiments, the anti-CD137L antibody comprises 1, 2, or 3 HVR sequences from a VH region comprising the amino acid sequence of SEQ ID NO:287 and/or 1, 2, or 3 HVR sequences from a VL region comprising the amino acid sequence of SEQ ID NO:288. In some embodiments, the anti-CD137L antibody comprises a heavy chain variable (VH) region comprising the amino acid sequence of SEQ ID NO:287 and/or a light chain variable (VL) region comprising the amino acid sequence of SEQ ID NO:288. In some embodiments, the anti-CD137L antibody comprises 1, 2, or 3 HVR sequences from a VH region comprising the amino acid sequence of SEQ ID NO:289 and/or 1, 2, or 3 HVR sequences from a VL region comprising the amino acid sequence of SEQ ID NO:290. In some embodiments, the anti-CD137L antibody comprises a heavy chain variable (VH) region comprising the amino acid sequence of SEQ ID NO:289 and/or a light chain variable (VL) region comprising the amino acid sequence of SEQ ID NO:290. In some embodiments, the anti-CD137L antibody comprises 1, 2, or 3 HVR sequences from a VH region comprising the amino acid sequence of SEQ ID NO:291 and/or 1, 2, or 3 HVR sequences from a VL region comprising the amino acid sequence of SEQ ID NO:292. In some embodiments, the anti-CD137L antibody comprises a heavy chain variable (VH) region comprising the amino acid sequence of SEQ ID NO:291 and/or a light chain variable (VL) region comprising the amino acid sequence of SEQ ID NO:292. In some embodiments, the anti-CD137L antibody comprises 1, 2, or 3 HVR sequences from a VH region comprising the amino acid sequence of SEQ ID NO:293 and/or 1, 2, or 3 HVR sequences from a VL region comprising the amino acid sequence of SEQ ID NO:294. In some embodiments, the anti-CD137L antibody comprises a heavy chain variable (VH) region comprising the amino acid sequence of SEQ ID NO:293 and/or a light chain variable (VL) region comprising the amino acid sequence of SEQ ID NO:294. In some embodiments, the anti-CD137L antibody comprises 1, 2, or 3 HVR sequences from a VH region comprising the amino acid sequence of SEQ ID NO:295 and/or 1, 2, or 3 HVR sequences from a VL region comprising the amino acid sequence of SEQ ID NO:296. In some embodiments, the anti-CD137L antibody comprises a heavy chain variable (VH) region comprising the amino acid sequence of SEQ ID NO:295 and/or a light chain variable (VL) region comprising the amino acid sequence of SEQ ID NO:296. In some embodiments, the anti-CD137L antibody comprises 1, 2, or 3 HVR sequences from a VH region comprising the amino acid sequence of SEQ ID NO:297 and/or 1, 2, or 3 HVR sequences from a VL region comprising the amino acid sequence of SEQ ID NO:298. In some embodiments, the anti-CD137L antibody comprises a heavy chain variable (VH) region comprising the amino acid sequence of SEQ ID NO:297 and/or a light chain variable (VL) region comprising the amino acid sequence of SEQ ID NO:298. In some embodiments, the anti-CD137L antibody comprises 1, 2, or 3 HVR sequences from a VH region comprising the amino acid sequence of SEQ ID NO:299 and/or 1, 2, or 3 HVR sequences from a VL region comprising the amino acid sequence of SEQ ID NO:300. In some embodiments, the anti-CD137L antibody comprises a heavy chain variable (VH) region comprising the amino acid sequence of SEQ ID NO:299 and/or a light chain variable (VL) region comprising the amino acid sequence of SEQ ID NO:300. In some embodiments, the anti-CD137L antibody comprises 1, 2, or 3 HVR sequences from a VH region comprising the amino acid sequence of SEQ ID NO:301 and/or 1, 2, or 3 HVR sequences from a VL region comprising the amino acid sequence of SEQ ID NO:302. In some embodiments, the anti-CD137L antibody comprises a heavy chain variable (VH) region comprising the amino acid sequence of SEQ ID NO:301 and/or a light chain variable (VL) region comprising the amino acid sequence of SEQ ID NO:302. In some embodiments, the anti-CD137L antibody comprises 1, 2, or 3 HVR sequences from a VH region comprising the amino acid sequence of SEQ ID NO:303 and/or 1, 2, or 3 HVR sequences from a VL region comprising the amino acid sequence of SEQ ID NO:304. In some embodiments, the anti-CD137L antibody comprises a heavy chain variable (VH) region comprising the amino acid sequence of SEQ ID NO:303 and/or a light chain variable (VL) region comprising the amino acid sequence of SEQ ID NO:304. In some embodiments, the anti-CD137L antibody comprises 1, 2, or 3 HVR sequences from a VH region comprising the amino acid sequence of SEQ ID NO:305 and/or 1, 2, or 3 HVR sequences from a VL region comprising the amino acid sequence of SEQ ID NO:306. In some embodiments, the anti-CD137L antibody comprises a heavy chain variable (VH) region comprising the amino acid sequence of SEQ ID NO:305 and/or a light chain variable (VL) region comprising the amino acid sequence of SEQ ID NO:306. In some embodiments, the anti-CD137L antibody comprises 1, 2, or 3 HVR sequences from a VH region comprising the amino acid sequence of SEQ ID NO:307 and/or 1, 2, or 3 HVR sequences from a VL region comprising the amino acid sequence of SEQ ID NO:308. In some embodiments, the anti-CD137L antibody comprises a heavy chain variable (VH) region comprising the amino acid sequence of SEQ ID NO:307 and/or a light chain variable (VL) region comprising the amino acid sequence of SEQ ID NO:308. In some embodiments, the anti-CD137L antibody comprises 1, 2, or 3 HVR sequences from a VH region comprising the amino acid sequence of SEQ ID NO:309 and/or 1, 2, or 3 HVR sequences from a VL region comprising the amino acid sequence of SEQ ID NO:310. In some embodiments, the anti-CD137L antibody comprises a heavy chain variable (VH) region comprising the amino acid sequence of SEQ ID NO:309 and/or a light chain variable (VL) region comprising the amino acid sequence of SEQ ID NO:310. In some embodiments, the anti-CD137L antibody comprises 1, 2, or 3 HVR sequences from a VH region comprising the amino acid sequence of SEQ ID NO:311 and/or 1, 2, or 3 HVR sequences from a VL region comprising the amino acid sequence of SEQ ID NO:312. In some embodiments, the anti-CD137L antibody comprises a heavy chain variable (VH) region comprising the amino acid sequence of SEQ ID NO:311 and/or a light chain variable (VL) region comprising the amino acid sequence of SEQ ID NO:312. In some embodiments, the anti-CD137L antibody comprises 1, 2, or 3 HVR sequences from a VH region comprising the amino acid sequence of SEQ ID NO:313 and/or 1, 2, or 3 HVR sequences from a VL region comprising the amino acid sequence of SEQ ID NO:314. In some embodiments, the anti-CD137L antibody comprises a heavy chain variable (VH) region comprising the amino acid sequence of SEQ ID NO:313 and/or a light chain variable (VL) region comprising the amino acid sequence of SEQ ID NO:314. In some embodiments, the anti-CD137L antibody comprises 1, 2, or 3 HVR sequences from a VH region comprising the amino acid sequence of SEQ ID NO:315 and/or 1, 2, or 3 HVR sequences from a VL region comprising the amino acid sequence of SEQ ID NO:316. In some embodiments, the anti-CD137L antibody comprises a heavy chain variable (VH) region comprising the amino acid sequence of SEQ ID NO:315 and/or a light chain variable (VL) region comprising the amino acid sequence of SEQ ID NO:316. In some embodiments, the anti-CD137L antibody comprises 1, 2, or 3 HVR sequences from a VH region comprising the amino acid sequence of SEQ ID NO:317 and/or 1, 2, or 3 HVR sequences from a VL region comprising the amino acid sequence of SEQ ID NO:318. In some embodiments, the anti-CD137L antibody comprises a heavy chain variable (VH) region comprising the amino acid sequence of SEQ ID NO:317 and/or a light chain variable (VL) region comprising the amino acid sequence of SEQ ID NO:318. In some embodiments, the anti-CD137L antibody comprises 1, 2, or 3 HVR sequences from a VH region comprising the amino acid sequence of SEQ ID NO:319 and/or 1, 2, or 3 HVR sequences from a VL region comprising the amino acid sequence of SEQ ID NO:320. In some embodiments, the anti-CD137L antibody comprises a heavy chain variable (VH) region comprising the amino acid sequence of SEQ ID NO:319 and/or a light chain variable (VL) region comprising the amino acid sequence of SEQ ID NO:320. In some embodiments, the anti-CD137L antibody comprises 1, 2, or 3 HVR sequences from a VH region comprising the amino acid sequence of SEQ ID NO:321 and/or 1, 2, or 3 HVR sequences from a VL region comprising the amino acid sequence of SEQ ID NO:322. In some embodiments, the anti-CD137L antibody comprises a heavy chain variable (VH) region comprising the amino acid sequence of SEQ ID NO:321 and/or a light chain variable (VL) region comprising the amino acid sequence of SEQ ID NO:322. In some embodiments, the anti-CD137L antibody comprises 1, 2, or 3 HVR sequences from a VH region comprising the amino acid sequence of SEQ ID NO:323 and/or 1, 2, or 3 HVR sequences from a VL region comprising the amino acid sequence of SEQ ID NO:324. In some embodiments, the anti-CD137L antibody comprises a heavy chain variable (VH) region comprising the amino acid sequence of SEQ ID NO:323 and/or a light chain variable (VL) region comprising the amino acid sequence of SEQ ID NO:324. In some embodiments, the anti-CD137L antibody comprises 1, 2, or 3 HVR sequences from a VH region comprising the amino acid sequence of SEQ ID NO:325 and/or 1, 2, or 3 HVR sequences from a VL region comprising the amino acid sequence of SEQ ID NO:326. In some embodiments, the anti-CD137L antibody comprises a heavy chain variable (VH) region comprising the amino acid sequence of SEQ ID NO:325 and/or a light chain variable (VL) region comprising the amino acid sequence of SEQ ID NO:326. In some embodiments, the anti-CD137L antibody comprises 1, 2, or 3 HVR sequences from a VH region comprising the amino acid sequence of SEQ ID NO:327 and/or 1, 2, or 3 HVR sequences from a VL region comprising the amino acid sequence of SEQ ID NO:328. In some embodiments, the anti-CD137L antibody comprises a heavy chain variable (VH) region comprising the amino acid sequence of SEQ ID NO:327 and/or a light chain variable (VL) region comprising the amino acid sequence of SEQ ID NO:328. In some embodiments, the anti-CD137L antibody comprises 1, 2, or 3 HVR sequences from a VH region comprising the amino acid sequence of SEQ ID NO:329 and/or 1, 2, or 3 HVR sequences from a VL region comprising the amino acid sequence of SEQ ID NO:330. In some embodiments, the anti-CD137L antibody comprises a heavy chain variable (VH) region comprising the amino acid sequence of SEQ ID NO:329 and/or a light chain variable (VL) region comprising the amino acid sequence of SEQ ID NO:330. In some embodiments, the anti-CD137L antibody comprises 1, 2, or 3 HVR sequences from a VH region comprising the amino acid sequence of SEQ ID NO:331 and/or 1, 2, or 3 HVR sequences from a VL region comprising the amino acid sequence of SEQ ID NO:332. In some embodiments, the anti-CD137L antibody comprises a heavy chain variable (VH) region comprising the amino acid sequence of SEQ ID NO:331 and/or a light chain variable (VL) region comprising the amino acid sequence of SEQ ID NO:332. In some embodiments, the anti-CD137L antibody comprises 1, 2, or 3 HVR sequences from a VH region comprising the amino acid sequence of SEQ ID NO:333 and/or 1, 2, or 3 HVR sequences from a VL region comprising the amino acid sequence of SEQ ID NO:334. In some embodiments, the anti-CD137L antibody comprises a heavy chain variable (VH) region comprising the amino acid sequence of SEQ ID NO:333 and/or a light chain variable (VL) region comprising the amino acid sequence of SEQ ID NO: 334. In some embodiments, the anti-CD137L antibody comprises 1, 2, or 3 HVR sequences from a VH region comprising the amino acid sequence of SEQ ID NO:335 and/or 1, 2, or 3 HVR sequences from a VL region comprising the amino acid sequence of SEQ ID NO:336. In some embodiments, the anti-CD137L antibody comprises a heavy chain variable (VH) region comprising the amino acid sequence of SEQ ID NO:335 and/or a light chain variable (VL) region comprising the amino acid sequence of SEQ ID NO:336.

In some embodiments, the anti-CD137L antibody comprises an Fc region. In some embodiments, the Fc region is a non-human Fc region. In some embodiments, the Fc region is a mouse Fc region. In some embodiments, the Fc region is a mouse IgG1, IgG2a, IgG2b, or IgG3 Fc region. In some embodiments, the Fc region is a rabbit Fc region, e.g., a rabbit IgG Fc region. In some embodiments, the Fc region is a rat Fc region, e.g., a rat IgG2b Fc region. In some embodiments, the Fc region is a chicken Fc region. A variety of Fc regions suitable, e.g., for IHC are known in the art and contemplated for use herein.

A CD137L antibody can be converted from one class or subclass to another class or subclass using methods known in the art. An exemplary method for producing an antibody in a desired class or subclass comprises the steps of isolating a nucleic acid encoding a heavy chain of an CD137L antibody and a nucleic acid encoding a light chain of a CD137L antibody, isolating the sequence encoding the V_{H} region, ligating the V_{H} sequence to a sequence encoding a heavy chain constant region of the desired class or subclass, expressing the light chain gene and the heavy chain construct in a cell, and collecting the CD137L antibody.

Further, the antibodies provided by the present disclosure can be monoclonal or polyclonal, but preferably monoclonal.

### Antigen Binding Fragments

In some aspects, the present disclosure provides antigen-binding fragments of any of the anti-CD137L antibodies provided by the present disclosure.

The antigen-binding fragment may comprise any sequences of the antibody. In some embodiments, the antigen-binding fragment comprises the amino acid sequence of: (1) a light chain of an anti-CD137L antibody; (2) a heavy chain of an anti-CD137L antibody; (3) a variable region from the light chain of an anti-CD137L antibody; (4) a variable region from the heavy chain of an anti-CD137L antibody; (5) one or more HVRs (two, three, four, five, or six HRVs) of an anti-CD137L antibody; or (6) three HVRs from the light chain and three HVRs from the heavy chain of an anti-CD137L antibody.

In some particular embodiments, the disclosure provides an antigen-binding fragment of an antibody selected from those listed in Tables 1-3.

In some other particular embodiments, the antigen-binding fragments of an anti-CD137L antibody include: (i) a Fab fragment, which is a monovalent fragment consisting of the V_{L}, V_{H}, C_{L} and C_{H}1 domains; (ii) a F(ab′)₂ fragment, which is a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody; (v) a dAb fragment (Ward et al., (1989) Nature 341:544-546), which consists of a V_{H} domain; (vi) an isolated CDR, and (vii) single chain antibody (scFv), which is a polypeptide comprising a V_{L} region of an antibody linked to a V_{H} region of an antibody. Bird et al., (1988) Science 242:423-426 and Huston et al., (1988) Proc. Natl. Acad. Sci. USA 85:5879-5883.

In some particular embodiments, the antigen-binding fragment is a Fab fragment selected from those listed in Table 1.

### Antibody Derivatives

The anti-CD137L antibodies as described herein may include any antibody derived from the anti-CD137L antibodies of the present disclosure.

In some further aspects, the present disclosure provides derivatives of any of the CD137L antibodies provided by the present disclosure.

In one aspect, the antibody derivative is derived from modifications of the amino acid sequences of an illustrative antibody (“parent antibody”) of the disclosure while conserving the overall molecular structure of the parent antibody amino acid sequence. Amino acid sequences of any regions of the parent antibody chains may be modified, such as framework regions, HVR regions, or constant regions. Types of modifications include substitutions, insertions, deletions, or combinations thereof, of one or more amino acids of the parent antibody. For example, in some embodiments, a CDR of the present disclosure (e.g., as listed in Table 1 or 3) comprises 1, 2, 3, or more substitutions (e.g., conservative substitutions).

Amino acid substitutions encompass both conservative substitutions and non-conservative substitutions. The term “conservative amino acid substitution” means a replacement of one amino acid with another amino acid where the two amino acids have similarity in certain physico-chemical properties such as polarity, charge, solubility, hydrophobicity, hydrophilicity, and/or the amphipathic nature of the residues involved. For example, substitutions typically may be made within each of the following groups: (a) nonpolar (hydrophobic) amino acids, such as alanine, leucine, isoleucine, valine, proline, phenylalanine, tryptophan, and methionine: (b) polar neutral amino acids, such as glycine, serine, threonine, cysteine, tyrosine, asparagine, and glutamine; (c) positively charged (basic) amino acids, such as arginine, lysine, and histidine; and (d) negatively charged (acidic) amino acids, such as aspartic acid and glutamic acid.

The modifications may be made in any positions of the amino acid sequences of the antibody, including the HVRs, framework regions, or constant regions. In one embodiment, the present disclosure provides an antibody derivative that contains the V_{H} and V_{L} HVR sequences of an illustrative antibody of this disclosure, yet contains framework sequences different from those of the illustrative antibody. Such framework sequences can be obtained from public DNA databases or published references that include germline antibody gene sequences. For example, germline DNA sequences for human heavy and light chain variable region genes can be found in the Genbank database or in the “VBase” human germline sequence database (Kabat, E. A., et al., Sequences of Proteins of Immunological Interest, Fifth Edition, U.S. Department of Health and Human Services, NIH Publication No. 91-3242 (1991); Tomlinson, I. M., et al., J. Mol. Biol. 227:776-798 (1992); and Cox, J. P. L. et al., Eur. J. Immunol. 24:827-836 (1994)). Framework sequences that may be used in constructing an antibody derivative include those that are structurally similar to the framework sequences used by illustrative antibodies of the disclosure, e.g., similar to the V_{H} 3-23 framework sequences and/or the V_{L} λ3 or λ1-13 framework sequences used by illustrative antibodies of the disclosure. For example, the HVR_H1, HVR_H2, and HVR H3 sequences, and the HVR_L1, HVR_L2, and HVR_L3 sequences of an illustrative antibody can be grafted onto framework regions that have the identical sequence as that found in the germline immunoglobulin gene from which the framework sequence derive, or the HVR sequences can be grafted onto framework regions that contain one or more mutations as compared to the germline sequences.

In a particular embodiment, the antibody derivative is a chimeric antibody which comprises an amino acid sequence of an illustrative antibody of the disclosure. In one example, one or more HVRs from one or more illustrative human antibodies are combined with HVRs from an antibody from a non-human animal, such as mouse or rat. In another example, all of the HVRs of the chimeric antibody are derived from one or more illustrative antibodies. In some particular embodiments, the chimeric antibody comprises one, two, or three HVRs from the heavy chain variable region or from the light chain variable region of an illustrative antibody. Chimeric antibodies can be generated using conventional methods known in the art.

Another type of modification is to mutate amino acid residues within the HRV regions of the V_{H} and/or V_{L} chain. Site-directed mutagenesis or PCR-mediated mutagenesis can be performed to introduce the mutation(s) and the effect on antibody binding, or other functional property of interest, can be evaluated in in vitro or in vivo assays known in the art. Typically, conservative substitutions are introduced. The mutations may be amino acid additions and/or deletions. Moreover, typically no more than one, two, three, four or five residues within a HVR region are altered. In some embodiments, the antibody derivative comprises 1, 2, 3, or 4 amino acid substitutions in the heavy chain HVRs and/or in the light chain HVRs. In another embodiment, the amino acid substitution is to change one or more cysteines in an antibody to another residue, such as, without limitation, alanine or serine. The cysteine may be a canonical or non-canonical cysteine. In one embodiment, the antibody derivative has 1, 2, 3, or 4 conservative amino acid substitutions in the heavy chain HVR regions relative to the amino acid sequences of an illustrative antibody.

Modifications may also be made to the framework residues within the V_{H} and/or V_{L} regions. Typically, such framework variants are made to decrease the immunogenicity of the antibody. One approach is to “back mutate” one or more framework residues to the corresponding germline sequence. An antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back mutated” to the germline sequence by, for example, site-directed mutagenesis or PCR-mediated mutagenesis.

In addition, modifications may also be made within the Fc region of an illustrative antibody, typically to alter one or more functional properties of the antibody, such as serum half-life, complement fixation, Fc receptor binding, and/or antigen-dependent cellular cytotoxicity. In one example, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody. In another case, the Fc hinge region of an antibody is mutated to decrease the biological half-life of the antibody.

Furthermore, an antibody of the disclosure may be modified to alter its potential glycosylation site or pattern in accordance with routine experimentation known in the art. In another aspect, the present disclosure provide an derivative of an anti-CD137L antibody of the disclosure that contains at least one mutation in an variable region of a light chain or heavy chain that changes the pattern of glycosylation in the variable region. Such an antibody derivative may have an increased affinity and/or a modified specificity for binding an antigen. The mutations may add a novel glycosylation site in the V region, change the location of one or more V region glycosylation site(s), or remove a pre-existing V region glycosylation site. In one embodiment, the present disclosure provides a derivative of an anti-CD137L antibody having a potential N-linked glycosylation site at asparagine in the heavy chain variable region, wherein the potential N-linked glycosylation site in one heavy chain variable region is removed. In another embodiment, the present disclosure provides a derivative of an anti-CD137L antibody having a potential N-linked glycosylation site at asparagine in the heavy chain variable region, wherein the potential N-linked glycosylation site in both heavy chain variable regions is removed. Method of altering the glycosylation pattern of an antibody is known in the art, such as those described in U.S. Pat. No. 6,933,368, the disclosure of which incorporated herein by reference.

In another aspect, the present disclosure provides an antibody derivative that comprises an anti-CD137L antibody, or antigen-binding fragment thereof, as described herein, linked to an additional molecular entity. Examples of additional molecular entities include pharmaceutical agents, peptides or proteins, detection agent or labels, and antibodies.

In some embodiments, the antibody derivative comprises an antibody of the disclosure linked to a pharmaceutical agent. Examples of pharmaceutical agents include cytotoxic agents or other cancer therapeutic agents, and radioactive isotopes. Specific examples of cytotoxic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Examples of radioactive isotopes that can be conjugated to antibodies for use diagnostically or therapeutically include, but are not limited to, iodine¹³¹, indium¹¹¹, yttrium⁹⁰ and lutetium¹⁷⁷. Methods for linking an antibody to a pharmaceutical agent are known in the art, such as using various linker technologies. Examples of linker types include hydrazones, thioethers, esters, disulfides and peptide-containing linkers. For further discussion of linkers and methods for linking therapeutic agents to antibodies, see also Saito et al., Adv. Drug Deliv. Rev. 55:199-215 (2003); Trail, et al., Cancer Immunol. Immunother. 52:328-337 (2003); Payne, Cancer Cell 3:207-212 (2003); Allen, Nat. Rev. Cancer 2:750-763 (2002); Pastan, I. and Kreitman, Curr. Opin. Investig. Drugs 3:1089-1091 (2002); Senter, P. D. and Springer, C. J. (2001) Adv. Drug Deliv. Rev. 53:247-264.

In a particular embodiment, the antibody derivative is an anti-CD137L antibody multimer, which is a multimeric form of an anti-CD137L antibody, such as antibody dimers, trimers, or higher-order multimers of monomeric antibodies. Individual monomers within an antibody multimer may be identical or different. In addition, individual antibodies within a multimer may have the same or different binding specificities. Multimerization of antibodies may be accomplished through natural aggregation of antibodies. For example, some percentage of purified antibody preparations (e.g., purified IgG4 molecules) spontaneously form protein aggregates containing antibody homodimers, and other higher-order antibody multimers. Alternatively, antibody homodimers may be formed through chemical linkage techniques known in the art, such as through using crosslinking agents. Suitable crosslinkers include those that are heterobifunctional, having two distinctly reactive groups separated by an appropriate spacer (such as m-maleimidobenzoyl-N-hydroxysuccinimide ester, succinimidyl 4-(maleimidomethyl)cyclohexane-1-carboxylate, and N-succinimidyl S-acethylthio-acetate) or homobifunctional (such as disuccinimidyl suberate). Such linkers are commercially available from, for example, Pierce Chemical Company, Rockford, Ill. Antibodies can also be made to multimerize through recombinant DNA techniques known in the art.

Examples of other antibody derivatives provided by the present disclosure include single chain antibodies, diabodies, domain antibodies, nanobodies, and unibodies. A “single-chain antibody” (scFv) consists of a single polypeptide chain comprising a V_{L} domain linked to a V_{H} domain wherein V_{L} domain and V_{H} domain are paired to form a monovalent molecule. Single chain antibody can be prepared according to method known in the art (see, for example, Bird et al., (1988) Science 242:423-426 and Huston et al., (1988) Proc. Natl. Acad. Sci. USA 85:5879-5883). A “diabody” consists of two chains, each chain comprising a heavy chain variable region connected to a light chain variable region on the same polypeptide chain connected by a short peptide linker, wherein the two regions on the same chain do not pair with each other but with complementary domains on the other chain to form a bispecific molecule. Methods of preparing diabodies are known in the art (See, e.g., Holliger P. et al., (1993) Proc. Natl. Acad. Sci. USA 90:6444-6448, and Poljak R. J. et al., (1994) Structure 2:1121-1123). Domain antibodies (dAbs) are small functional binding units of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640; WO05/035572, WO04/101790, WO04/081026, WO04/058821, WO04/003019 and WO03/002609). Nanobodies are derived from the heavy chains of an antibody. A nanobody typically comprises a single variable domain and two constant domains (CH2 and CH3) and retains antigen-binding capacity of the original antibody. Nanobodies can be prepared by methods known in the art (See e.g., U.S. Pat. Nos. 6,765,087, 6,838,254, WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

Detecting CD37L Expression

In some embodiments, the present disclosure relates to methods of detecting the level of CD137L expression in a sample. In some embodiments, measuring the level of expression of CD137L in a sample comprises measuring the level of protein expression of CD137L. In some embodiments, the methods comprise obtaining a human tissue sample (e.g., from a tumor, such as from tumor biopsy). In some embodiments, the methods comprise contacting a human tissue sample (e.g., from a tumor, such as from tumor biopsy) with an anti-CD137L antibody or antigen-binding fragment of the present disclosure.

In some embodiments, the methods comprise detecting binding of an anti-CD137L antibody or antigen-binding fragment of the present disclosure to a human tissue sample (e.g., from a tumor, such as from tumor biopsy). In some embodiments, the level of expression of CD137L in a sample is measured by determining the level of protein expression of CD137L. Suitable methods of measuring protein expression in a sample are known in the art, including, for example, immunoassays, immunohistochemistry (IHC), PET imaging, Western blotting, enzyme-linked immunosorbent assays (ELISAs), flow cytometry, and mass spectrometry. In some embodiments, the level of CD137L protein expression is measured by immunoassay, Western blotting, ELISA, IHC, and/or flow cytometry. In some embodiments, the sample is a fixed tissue sample, e.g., an FFPE sample. In some embodiments, the sample comprises one or more cancer cells. In some embodiments, the level of expression of CD137L in the sample is the level of CD137L expression by cancer cells. In some embodiments, binding of the antibody or antigen-binding fragment to the sample indicates the level of expression of human CD137L in the sample.

Subjects

In some embodiments, the present disclosure relates to subjects suffering from or believed to be suffering from cancer. In some embodiments, the subject has been diagnosed with cancer. In some embodiments, the subject has not been diagnosed with cancer. In some embodiments, the subject is at risk of developing cancer.

The subject may be suffering from, or believed to be suffering from, any cancer known in the art, including, for example, lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), mesothelioma, and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; adrenal cancers (e.g., adrenocortical carcinoma); cancers in digestive system such as esophageal carcinoma, leiomyoma, epidermoid carcinoma, adenocarcinoma, leiomyosarcoma, stomach adenocarcinomas, intestinal lipomas, intestinal neurofibromas, intestinal fibromas, polyps in large intestine, colon and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal papillary cell carcinoma, renal cell carcinoma, renal clear cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblastic, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, Diffused Large B cell lymphoma, T cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, germ cell tumors, and choriocarcinoma); breast cancer; female reproductive system cancers such as uterine cancer (endometrial carcinoma, uterine carcinosarcoma), cervical cancer (cervical carcinoma), cancer of the ovaries (ovarian carcinoma, ovarian serous cystadenocarcinoma), vulvar carcinoma, vaginal carcinoma, fallopian tube cancer, and hydatidiform mole; thyroid cancer (including papillary, follicular, anaplastic, or medullary cancer) and thymoma; pheochromocytomas (adrenal gland) and paraganglioma; noncancerous growths of the parathyroid glands; and pancreatic cancers.

In some embodiments, the subject has not previously received one or more anti-cancer therapies. In some embodiments, the subject has previously received and/or is currently receiving one or more anti-cancer therapies.

Samples Obtained from a Subject

In some embodiments, the present disclosure relates to measuring the level of CD137L expression in a sample obtained from a subject. In some embodiments, the level of expression of CD137L is measured in one or more (e.g., one or more, two or more, three or more, four or more, etc.) samples obtained from a subject. Any suitable sample in the form of tissues and/or fluids that are known or believed to contain diseased cells and/or the target of interest (e.g., full length CD137L, CD137L fragments including soluble CD137L fragments) may be used in the methods described herein, including, for example, sputum, pleural fluid, lymph fluid, bone marrow, blood, plasma, serum, urine, tissue samples (samples known or expected to contain cancer cells), tumor samples, tumor biopsies, etc. In some embodiments, the sample is a serum sample. In some embodiments, the sample is a tumor sample. In some embodiments, the sample is a tumor biopsy. In some embodiments, the sample comprises one or more cancer cells.

Methods of obtaining suitable tissue and/or fluid samples (e.g., methods that are appropriate for obtaining a representative sample from a particular type, location, disease tissue, etc.) are well known to one of ordinary skill in the art, including, for example, by resection, bone marrow biopsy or bone marrow aspiration, endoscopic biopsy or endoscopic aspiration (e.g., cystoscopy, bronchoscopy, colonoscopy, etc.), needle biopsy or needle aspiration (e.g., fine needle aspiration, core needle biopsy, vacuum-assisted biopsy, image-guided biopsy, etc.) skin biopsy (e.g., shave biopsy, punch biopsy, incisional biopsy, excisional biopsy, etc.), various other surgical tissue (e.g., tumor tissue) biopsy and/or excision strategies, and fluid collections (e.g., collecting urine, blood, serum, plasma, sputum, etc.).

In some embodiments, the one or more samples obtained from the subject are enriched for diseased (e.g., cancerous) cells. Methods of enriching a tissue or fluid preparation for diseased (e.g., cancerous) cells are known in the art, including, for example, by separating diseased (e.g., cancerous) cells from normal cells by flow cytometry. In some embodiments, the level of expression of CD137L is measured in the enriched samples. In some embodiments, the level of expression of CD137L is measured in samples that have not been enriched or otherwise altered after isolation.

In some embodiments, the one or more samples are fixed (i.e. preserved) by conventional methodology (See e.g., “Manual of Histological Staining Method of the Armed Forces Institute of Pathology,” 3^{rd} edition (1960) Lee G. Luna, H T (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). The choice of a fixative may be determined by one of ordinary skill in the art for the purpose for which the sample is to be analyzed. The length of fixation will depend upon the size and type of the tissue sample and the fixative used (e.g., neutral buffered formalin, paraformaldehyde, etc.), as will be appreciated by one of ordinary skill in the art. In some embodiments, the level of expression of CD137L is measured in a sample that is fixed. In some embodiments, the level of expression of CD137L is measured in samples that have not been fixed or otherwise altered after isolation.

In some embodiments, one or more samples are obtained from the subject prior to administration with an anti-cancer therapy (e.g., an anti-CD137 antibody therapy and/or a checkpoint blockade immunotherapy). In some embodiments, one or more samples are obtained from the subject after administration of a first and/or subsequent does of an anti-cancer therapy (e.g., an anti-CD137 antibody therapy and/or a checkpoint blockade immunotherapy). In some embodiments, one or more samples are obtained from the subject after completion of an anti-cancer therapy regimen (e.g., an anti-CD137 antibody therapy and/or a checkpoint blockade immunotherapy). In some embodiments, one or more samples are obtained from the subject, prior to, during, and after completion of an anti-cancer therapy regimen (e.g., an anti-CD137 antibody therapy and/or a checkpoint blockade immunotherapy).

Comparison to a Reference Level

In some embodiments, the present disclosure relates to comparing the level of expression of CD137L in a sample obtained from a subject to a reference level of expression of CD137L. In some embodiments, the reference level is the level of expression of CD137L in a reference sample (e.g., a reference cell (such as a cell line, including but not limited to Raji (ATCC, CC-86) or Daudi (ATCC, CCL-213) cell lines), a corresponding sample taken from one or more patients determined to be responsive to anti-CD137 antibody therapy, a corresponding sample taken from one or more patients determined to be non-responsive to anti-CD137 antibody therapy, a corresponding adjacent normal tissue, etc.). In some embodiments, the reference level is measured in the reference sample using the same method as was used to measure the level of expression of CD137L in the subject's sample. In some embodiments, the reference level is measured in the reference sample using a different method than was used to measure the level of expression of CD137L in the subject's sample.

Without wishing to be bound to theory, it is thought that anti-CD137 antibody treatment may not lead to downregulation of CD137 on immune cells as significantly as the CD137L:CD137 interaction. As such, a tumor or cancer cell that has low expression of CD137L may interact with anti-tumor T cells that have higher CD137 (e.g., as compared to a tumor or cancer cell with higher levels of CD137L expression that cause downregulation of CD137 in interacting cells). In addition, the use of an anti-CD137 agonist antibody that blocks CD137L binding to CD137 may be advantageous, in that it stimulates CD137 signaling while blocking CD137L-mediated downregulation of CD137 on responding cells.

In some embodiments, the reference level is the level of expression of CD137L (e.g., average level of expression) on one or more reference cells. In some embodiments, the one or more reference cells are cells taken from a diseased tissue isolated from a cancer patient (e.g., one or more cancer cells from a patient suffering from adrenocortical carcinoma, bladder urothelial carcinoma, breast invasive carcinoma, cervical or endocervical cancers, cholangiocarcinoma, colon adenocarcinoma, colorectal adenocarcinoma, lymphoid neoplasm diffused large-B cell lymphoma, esophageal carcinoma, glioblastoma multiforme and/or low grade glioma, head and Neck squamous cell carcinoma, kidney chromophobe, kidney renal clear cell carcinoma, kidney renal papillary cell carcinoma, acute myeloid leukemia, low grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma or paraganglioma, prostate adenocarcinoma, rectum adenocarcinoma, sarcoma, skin cutaneous melanoma, stomach adenocarcinoma, stomach or esophageal carcinoma, testicular germ cell tumors, thyroid carcinoma, thymoma, uterine corpus endometrial carcinoma, uterine carcinosarcoma, uveal melanoma, etc.). In some embodiments, the one or more reference cells are one or more cells from a cancer cell line (e.g., a liver cancer cell line, a colon cancer cell line, a melanoma cell line, a lung cancer cell line, a pancreatic cancer cell line, a prostate cancer cell line, a B cell lymphoma cell line, a T cell lymphoma cell line, etc.). In some embodiments, the one or more reference cells are one or more cells of an adjacent normal tissue in the subject (e.g., comparing CD137L expression (such as by immunohistochemical staining) in a tumor sample from the patient to CD137L expression in a normal tissue adjacent to the tumor, etc.).

In some embodiments, the reference level is the level of expression of CD137L in one or more samples isolated from one or more patients determined to be responsive to anti-CD137 antibody therapy (e.g., one or more samples isolated from one or more patients determined by a clinician to be responsive to anti-CD137 antibody therapy (such as patients receiving treatment with an anti-CD137 antibody in a clinical trial)). In some embodiments, the reference level is the level of expression of CD137L in one or more samples isolated from one or more patients determined to be non-responsive to anti-CD137 antibody therapy (e.g., one or more samples isolated from one or more patients determined by a clinician to be non-responsive to anti-CD137 antibody therapy (such as patients receiving treatment with an anti-CD137 antibody in a clinical trial)).

In some embodiments, the reference level is a pre-determined level of CD137L expression (e.g., the average level of expression of CD137L in a database of diseased samples (such as tissue biopsies or serum samples) isolated from multiple reference patients; the average level of expression of CD137L in a database of samples (such as tissue biopsies or serum samples) isolated from multiple healthy reference patients; etc.).

In some embodiments, the reference level of expression of CD137L refers to a detectable level of expression. That is to say, in some embodiments, the level of expression of CD137L measured in the sample obtained from the subject is considered to be lower than a reference level when the level of expression of CD137L in the sample is undetectable, e.g., below the limit of detection.

In some embodiments, the level of expression of CD137L measured in the sample obtained from the subject is considered to be lower than the reference level when the level of expression of CD137L in the sample is at least about 25% lower than the reference level. For example, the level of expression of CD137L measured in the sample obtained from the subject is considered to be lower than the reference level when the level of expression of CD137L in the sample is at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% lower than the reference level. In some embodiments, the level of expression of CD137L measured in the sample obtained from the subject is considered to be lower than the reference level when the level of expression of CD137L in the sample is at least about 1-fold lower than the reference level. For example, the level of expression of CD137L measured in the sample obtained from the subject is considered to be lower than the reference level when the level of expression of CD137L in the sample is at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5 fold, at least about 8-fold, at least about 8.5-fold, at least about 9-fold, at least about 9.5-fold, at least about 10-fold, at least about 100-fold, or at least about 1000-fold lower than the reference level. In some embodiments, the level of expression of CD137L in the sample obtained from the subject is below the limit of detection. In some embodiments, the level of expression of CD137L measured in the sample obtained from the subject is considered to be lower than the reference level when the level of expression of CD137L in the sample is below the limit of detection while the reference level is above the limit of detection, is detectable, and/or is not zero. In some embodiments, a level is considered to be below the limit of detection when the level does not give an appreciable signal, a detectable signal, and/or is not significantly different than an appropriate negative control when performing an assay for measuring the level of CD137L expression (e.g., below the limit of detection of an assay measuring RNA transcript expression of CD137L (such as RT-PCR, in situ hybridization, and/or next generation sequencing), below the limit of detection of an assay measuring CD137L protein expression (such as an immunoassay, PET imaging, Western blotting, ELISA, immunohistochemistry, and/or flow cytometry), etc.).

In some embodiments, a subject is administered an effective amount of an anti-CD137 antibody when the level of expression of CD137L in a sample obtained from the subject is lower than the reference level. In some embodiments, a subject is determined to be likely to respond to an anti-CD137 antibody when the level of expression of CD137L in a sample obtained from the subject is lower than the reference level. In some embodiments, a subject is administered an effective amount of an anti-CD137 antibody after the subject has been determined to be likely to respond to the anti-CD137 antibody. In some embodiments, a subject having cancer is selected for treatment with an anti-CD137 antibody when the level of expression of CD137L in a sample obtained from the subject is lower than the reference level. In some embodiments, a subject is positively stratified for enrollment into an anti-CD137 antibody therapy when the level of expression of CD137L in a sample obtained from the subject is lower than the reference level.

In some embodiments, the level of expression of CD137L measured in the sample obtained from the subject is considered to be higher than the reference level when the level of expression of CD137L in the sample is at least about 5% higher than the reference level. For example, the level of expression of CD137L measured in the sample obtained from the subject is considered to be higher than the reference level when the level of expression of CD137L in the sample is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% higher than the reference level. In some embodiments, the level of expression of CD137L measured in the sample obtained from the subject is considered to be higher than the reference level when the level of expression of CD137L in the sample is at least about 1-fold higher than the reference level. For example, the level of expression of CD137L measured in the sample obtained from the subject is considered to be higher than the reference level when the level of expression of CD137L in the sample is at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5 fold, at least about 8-fold, at least about 8.5-fold, at least about 9-fold, at least about 9.5-fold, at least about 10-fold, at least about 100-fold, or at least about 1000-fold higher than the reference level. In some embodiments, the level of expression of CD137L in the reference sample is below the limit of detection. In some embodiments, the level of expression of CD137L measured in the sample obtained from the subject is considered to be higher than the reference level when the level of expression of CD137L in the sample is above the limit of detection, is detectable, and/or is not zero while the level of expression of CD137L in the reference sample is below the limit of detection. In some embodiments, a level is considered to be below the limit of detection when the level does not give an appreciable signal, a detectable signal, and/or is not significantly different than an appropriate negative control when performing an assay for measuring the level of CD137L expression (e.g., below the limit of detection of an assay measuring RNA transcript expression of CD137L (such as RT-PCR, in situ hybridization, and/or next generation sequencing), below the limit of detection of an assay measuring CD137L protein expression (such as an immunoassay, PET imaging, Western blotting, ELISA, immunohistochemistry, and/or flow cytometry), etc.).

In some embodiments, a subject is administered an effective amount of a checkpoint blockade immunotherapy when the level of expression of CD137L in a sample obtained from the subject is higher than the reference level. In some embodiments, a subject is determined to be likely to respond to a checkpoint blockade immunotherapy when the level of expression of CD137L in a sample obtained from the subject is higher than the reference level. In some embodiments, a subject is administered an effective amount of a checkpoint blockade immunotherapy after the subject has been determined to be likely to respond to the checkpoint blockade immunotherapy. In some embodiments, a subject having cancer is selected for treatment with a checkpoint blockade immunotherapy when the level of expression of CD137L in a sample obtained from the subject is higher than the reference level. In some embodiments, a subject is positively stratified for enrollment into a checkpoint blockade immunotherapy when the level of expression of CD137L in a sample obtained from the subject is higher than the reference level. In some embodiments, a subject is negatively stratified for enrollment into an anti-CD137 antibody therapy when the level of expression of CD137L in a sample obtained from the subject is higher than the reference level.

In some embodiments, an anti-CD137 agonist antibody activates or enhances one or more activities of CD137. In some embodiments, an anti-CD137 agonist antibody demonstrates one or more of the following properties (e.g., in an in vitro assay using a CD137-expressing cell, such as a T cell)): stimulates T cell (e.g., CD8+ T cell) proliferation, induces cytokine (e.g., IFN-γ) secretion by T cells, and induces NFκB activation (e.g., in an NFκB reporter assay).

CD137L Expression and Anti-CD137 Antibody Therapies

In some embodiments, the present disclosure relates to methods of treating or delaying progression of cancer in a subject in need thereof comprising administering an effective amount of an anti-CD137 antibody to the subject if the level of expression of CD137L in a sample obtained from the subject is lower than a reference level. In some embodiments, the method comprises obtaining a sample from the subject, and measuring the level of expression of CD137L in the sample prior to administration of the anti-CD137 antibody. In some embodiments, the level of expression of CD137L in the sample obtained from the subject is below the limit of detection. In some embodiments, the subject is administered the anti-CD137 antibody when CD137L expression is below the limit of detection. In some embodiments, the anti-CD137 antibody is any one or more of the anti-CD137 antibodies described herein. In some embodiments, the level of expression of CD137L in the sample is determined using one of the anti-CD137L antibodies described herein, e.g., via IHC.

In some embodiments, the present disclosure relates to methods of determining whether a subject is likely to respond to an anti-CD137 antibody. In some embodiments, the method comprises obtaining a sample from the subject, measuring the level of expression of CD137L in the sample, and determining that the subject is likely to respond to the anti-CD137 antibody when the level of expression of CD137L in the sample is lower than a reference level. In some embodiments, the level of expression of CD137L in the sample obtained from the subject is below the limit of detection. In some embodiments, the subject is determined to be likely to respond to the anti-CD137 antibody when the CD137L expression is below the limit of detection. In some embodiments, the level of expression of CD137L in the sample is determined using one of the anti-CD137L antibodies described herein, e.g., via IHC.

In some embodiments, the present disclosure relates to methods of treating or delaying progression of cancer in a subject in need thereof comprising administering an effective amount of an anti-CD137 antibody to the subject after it is determined that the subject is likely to respond to the anti-CD137 antibody, based on expression of CD137L in a sample obtained from the subject. In some embodiments, the anti-CD137 antibody is any one or more of the anti-CD137 antibodies described herein. In some embodiments, responsiveness of the subject comprises treatment efficacy. In some embodiments, responsiveness of the subject comprises reduced tumor volume. In some embodiments, responsiveness of the subject comprises serological responsiveness.

In some embodiments, the present disclosure relates to methods of selecting a subject having cancer for treatment with an anti-CD137 antibody. In some embodiments, the method comprises measuring the level of expression of CD137L in a sample obtained from the subject, and selecting the subject for treatment with the anti-CD137 antibody if the level of expression of CD137L in the sample is lower than a reference level. In some embodiments, the level of expression of CD137L in the sample obtained from the subject is below the limit of detection. In some embodiments, the subject is selected for treatment with the anti-CD137 antibody when the CD137L expression is below the limit of detection. In some embodiments, the anti-CD137 antibody is any one or more of the anti-CD137 antibodies described herein. In some embodiments, the level of expression of CD137L in the sample is determined using one of the anti-CD137L antibodies described herein, e.g., via IHC.

Certain aspects of the present disclosure relate to anti-CD137 antibodies and antigen-binding fragments thereof, e.g., for use in the methods of treatment and related uses disclosed herein. In some embodiments, the anti-CD137 antibody binds to an extracellular domain of human CD137. In some aspects, the isolated anti-CD137 antibody binds to human CD137 at an epitope within amino acid residues 34-108 or 34-93 of SEQ ID NO.: 337.

(SEQ ID NO: 337)
MGNSCYNIVATLLLVLNFERTRSLQDPCSNCPAGT
ECDNNRNQICSPCPPNSFSSAGGQRTCDICRQCKG
VERTRKECSSTSNAECDCTPGFHCLGAGCSMCEQD
CKQGQELTKKGCKDCCEGTENDQKRGICRPWTNCS
LDGKSVLVNGTKERDVVCGPSPADLSPGASSVTPP
APAREPGHSPQIISFFLALTSTALLFLLFFLTLRF
SVVKRGRKKLLYIFKQPFMRPVQTTQEEDGCSCRE
TEEEEGGCEL

In some embodiments, the anti-CD137 antibody binds to one or more amino acid residues selected from the group consisting of amino acid residues 34-36, 53-55, and 92-93 of SEQ ID NO:337. In some embodiments, the anti-CD137 antibody binds to one or more of amino acid residues 34-36, one or more of amino acid residues 53-55, and one or more or amino acid residues 92-93 of SEQ ID NO:337. In some embodiments, the anti-CD137 antibody does not bind to one or more of amino acid residues selected from the group consisting of amino acid residues 109-112, 125, 126, 135-138, 150 and 151 of SEQ ID NO:337. In some embodiments, the anti-CD137 antibody does not bind to amino acid residues 109-112, 125, 126, 135-138, 150 and 151 of SEQ ID NO:337.

The antibody, in some embodiments, binds human CD137 with a K_{D} of 50 nM or less as measured by surface plasmon resonance. In certain embodiments, the antibody can be cross-reactive with at least one non-human species selected from the list consisting of cynomolgus monkey, mouse, rat and dog.

In some embodiments, the anti-CD137 antibody comprises an antibody heavy chain variable region comprising an HVR-H1 comprising the amino acid sequence FSLSTGGVGVGWI (SEQ ID NO: 338), an HVR-H2 comprising the amino acid sequence LALIDWADDKYYSPSLKSRL (SEQ ID NO:339), and an HVR-H3 comprising the amino acid sequence ARGGSDTVIGDWFAY (SEQ ID NO: 340), and an antibody light chain variable region comprising an HVR-L1 comprising the amino acid sequence RASQSIGSYLA (SEQ ID NO: 341), an HVR-L2 comprising the amino acid sequence DASNLETGV (SEQ ID NO: 342), and an HVR-L3 comprising the amino acid sequence YCQQGYYLWT (SEQ ID NO: 343). In some embodiments, the anti-CD137 antibody comprises an antibody heavy chain variable region comprising the amino acid sequence of SEQ ID NO: 344 or a sequence having at least 90% (e.g., 91%, 92%, 93%, 94%, 95%, 96%, 97%, 98% or 99%) to the sequence of SEQ ID NO: 344; and/or an antibody light chain variable region comprising the amino acid sequence of SEQ ID NO: 345 or a sequence having at least 90% (e.g., 91%, 92%, 93%, 94%, 95%, 96%, 97%, 98% or 99%) to the sequence of SEQ ID NO: 345.

(SEQ ID NO: 344)
EVQLVESGGGLVQPGGSLRLSCAASGFSL
STGGVGVGWIRQAPGKGLEWLALIDWADD
KYYSPSLKSRLTISRDNSKNTLYLQLNSL
RAEDTAVYYCARGGSDTVIGDWFAYWGQG
TLVTVSS
(SEQ ID NO: 345)
DIQLTQSPSSLSASVGDRVTTTCRASQSI
GSYLAWYQQKPGKAPKLLIYDASNLETGV
PSRFSGSGSGTDFTLTISSLQPEDFATYY
CQQGYYLWTFGQGTKVEIK

In some embodiments, the anti-CD137 antibody cross-competes with, or binds the same epitope as, an anti-CD137 antibody comprising: an antibody heavy chain variable region comprising an HVR-H1 comprising the amino acid sequence FSLSTGGVGVGWI (SEQ ID NO: 338), an HVR-H2 comprising the amino acid sequence LALIDWADDKYYSPSLKSRL (SEQ ID NO:339), and an HVR-H3 comprising the amino acid sequence ARGGSDTVIGDWFAY (SEQ ID NO: 340), and an antibody light chain variable region comprising an HVR-L1 comprising the amino acid sequence RASQSIGSYLA (SEQ ID NO: 341), an HVR-L2 comprising the amino acid sequence DASNLETGV (SEQ ID NO: 342), and an HVR-L3 comprising the amino acid sequence YCQQGYYLWT (SEQ ID NO: 343). In some embodiments, the anti-CD137 antibody cross-competes with, or binds the same epitope as, an anti-CD137 antibody comprising: an antibody heavy chain variable region comprising the amino acid sequence of SEQ ID NO: 344 and an antibody light chain variable region comprising the amino acid sequence of SEQ ID NO: 345.

Any of the anti-CD137 antibodies described in International Pub. No. WO2019037711 may find use in the methods of the present disclosure.

In some embodiments, the anti-CD137 antibody is utomilumab (PF-05082566; Pfizer). In some embodiments, the anti-CD137 antibody is urelumab (BMS-663513; Bristol-Myers Squibb). The sequences and structures of these antibodies are known in the art; see. e.g., Chin, S. M. et al. (2018) Nat. Commun. 9:4679.

In some embodiments, the anti-CD137 antibodies described herein have agonist activity on human CD137. In some embodiments, the anti-CD137 antibodies induce one or more (e.g., one or more, two or more, three or more, etc.) activities of human CD137 when a cell (e.g., a human cell) expressing human CD137 is contacted by the anti-CD137 antibody Various CD137 activities are known in the art and may include, without limitation, induction of NF-κB-dependent transcription, induction of T cell proliferation, prolonging T cell survival, co-stimulation of activated T cells, induction of cytokine secretion (such as IL-2), and induction of monocyte activation. In some embodiments, the one or more CD137 activities is not CD137 binding to its ligand. Methods of measuring CD137 activity (e.g., the induction of NF-κB-dependent transcription and/or T cell proliferation, etc.) are known in the art. In some embodiments, the anti-CD137 antibodies increase NF-κB dependent transcription in cells (e.g., human cells) expressing human CD137. In some embodiments, NF-κB dependent transcription is increased by about 10% or more, about 20% or more, about 30% or more, about 40% or more, about 50% or more, about 60% or more, about 70% or more, about 80% or more, about 90% or more, or about 99% or more in cells (e.g., human cells) expressing CD137 contacted with the anti-CD137 antibody, relative to a corresponding cell not contacted with the antibody. In some embodiments, NF-κB dependent transcription is increased by about 2-fold, 3-fold, 4-fold, 5-fold, 6-fold, 7-fold, 8-fold, 9-fold, 10-fold, 100-fold, 1000-fold or more in cells (e.g., human cells) expressing CD137 contacted with the anti-CD137 antibodies, relative to a corresponding cell not contacted with the antibody.

Antibody Production

Antibodies of the present disclosure may be produced using recombinant methods and compositions, e.g., as described in U.S. Pat. No. 4,816,567. In some embodiments, isolated nucleic acids encoding any antibody described herein are provided. Such nucleic acids may encode an amino acid sequence comprising the V_{L} and/or an amino acid sequence comprising the V_{H} of the antibodies (e.g., the light and/or heavy chains of the antibodies). In some embodiments, one or more vectors (e.g., expression vectors) comprising such nucleic acids are provided herein. In some embodiments, a host cell comprising such nucleic acids is provided. In one such embodiment, a host cell comprises (e.g., has been transformed with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In some embodiments, the host cell is eukaryotic, e.g. a Chinese Hamster Ovary (CHO) cell or a lymphoid cell (e.g., Y0, NS0, Sp20 cells). In some embodiments, antibodies of the present disclosure are produced in CHO cells. In some embodiments, antibodies of the present disclosure are modified, and do not include a C-terminal lysine residue (e.g., the C-terminal lysine residue of an antibody heavy chain described herein is removed (such as before or during antibody production)). In some embodiments, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of antibodies of the present disclosure, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and may be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Hybridoma production is a very well-established procedure. The common animal system for preparing hybridomas is the murine system. Immunization protocols and techniques for isolation of immunized splenocytes for fusion are known in the art. Fusion partners (e.g., murine myeloma cells) and fusion procedures are also known. One well-known method that may be used for making antibodies provided by the present disclosure involves the use of a XenoMouse™ animal system. XenoMouse™ mice are engineered mouse strains that comprise large fragments of human immunoglobulin heavy chain and light chain loci and are deficient in mouse antibody production. See, e.g., Green et al., Nature Genetics 7:13-21 (1994) and WO2003/040170. For example, the animal is immunized with a CD137L antigen. The CD137L antigen is isolated and/or purified CD137L, preferably CD137L. It may be a fragment of CD137L, such as the extracellular domain of CD137L. Immunization of animals can be carried out by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane, supra, and U.S. Pat. No. 5,994,619. The CD137L antigen may be administered with an adjuvant to stimulate the immune response. Exemplary adjuvants include complete or incomplete Freund's adjuvant, RIBI (muramyl dipeptides) or ISCOM (immunostimulating complexes). After immunization of an animal with a CD137L antigen, antibody-producing immortalized cell lines are prepared from cells isolated from the immunized animal. After immunization, the animal is sacrificed and lymph node and/or splenic B cells are immortalized. Methods of immortalizing cells include, but are not limited to, transferring them with oncogenes, inflecting them with the oncogenic virus cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line). Immortalized cells are screened using CD137L, a portion thereof, or a cell expressing CD137L. CD137L antibody-producing cells, e.g., hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

CD37L Expression and Checkpoint Blockade Immunotherapies

In some embodiments, the present disclosure relates to methods of treating or delaying progression of cancer in a subject in need thereof comprising administering an effective amount of a checkpoint blockade immunotherapy to the subject if the level of expression of CD137L in a sample obtained from the subject is higher than a reference level. In some embodiments, the method comprises obtaining a sample from the subject, and measuring the level of expression of CD137L in the sample prior to administration of the checkpoint blockade immunotherapy. In some embodiments, the checkpoint blockade immunotherapy is any one or more of the checkpoint blockade immunotherapies described herein. In some embodiments, the level of expression of CD137L in the sample is determined using one of the anti-CD137L antibodies described herein, e.g., via IHC.

In some embodiments, the present disclosure relates to methods of determining whether a subject is likely to respond to a checkpoint blockade immunotherapy. In some embodiments, the method comprises obtaining a sample from the subject, measuring the level of expression of CD137L in the sample, and determining that the subject is likely to respond to the checkpoint blockade immunotherapy when the level of expression of CD137L in the sample is higher than a reference level. In some embodiments, the level of expression of CD137L in the sample is determined using one of the anti-CD137L antibodies described herein, e.g., via IHC.

In some embodiments, the present disclosure relates to methods of treating or delaying progression of cancer in a subject in need thereof comprising administering an effective amount of a checkpoint blockade immunotherapy to the subject after it is determined that the subject is likely to respond to the checkpoint blockade immunotherapy. In some embodiments, the checkpoint blockade immunotherapy is any one or more of the checkpoint blockade immunotherapies described herein. In some embodiments, responsiveness of the subject comprises treatment efficacy. In some embodiments, responsiveness of the subject comprises reduced tumor volume. In some embodiments, responsiveness of the subject comprises serological responsiveness.

In some embodiments, the present disclosure relates to methods of selecting a subject having cancer for treatment with a checkpoint blockade immunotherapy. In some embodiments, the method comprises measuring the level of expression of CD137L in a sample obtained from the subject, and selecting the subject for treatment with the checkpoint blockade immunotherapy if the level of expression of CD137L in the sample is higher than a reference level. In some embodiments, the checkpoint blockade immunotherapy is any one or more of the checkpoint blockade immunotherapies described herein. In some embodiments, the level of expression of CD137L in the sample is determined using one of the anti-CD137L antibodies described herein, e.g., via IHC.

Checkpoint Blockade Immunotherapy

In some embodiments, the present disclosure relates to the use of a checkpoint blockade immunotherapy. In some embodiments, use of a checkpoint blockade immunotherapy comprises administering to a subject an antibody targeting one or more immune checkpoint pathways (e.g., the PD-1:PD-L1 pathway). Any checkpoint blockade immunotherapy known in the art may be used in the methods of the present disclosure, including, for example, a therapy comprising one or more antibodies targeting CTLA4, PD-1, PD-L1, TIM3, LAG3, CD27, CD28, CD40, OX40, GITR, BTLA, VISTA, B7-H3, B7-H4, IDO, and/or KIR. In some embodiments, the checkpoint blockade immunotherapy comprises administering an anti-PD-1 antibody. In some embodiments, the checkpoint blockade immunotherapy comprises administering an anti-PD-L1 antibody. In some embodiments, the checkpoint blockade immunotherapy is used in combination with an anti-CD137 antibody (as described herein).

### Additional Therapeutic Agents

The anti-cancer therapies described herein (e.g., an anti-CD137 antibody, a checkpoint blockade immunotherapy) may be administered alone as monotherapy, or may comprise one or more additional therapeutic agents or therapies. In some embodiments, the one or more (e.g., one or more, two or more, three or more, four or more, five or more, etc.) additional therapeutic agents are one or more of a viral gene therapy, immune checkpoint inhibitors, target therapies, radiation therapies, and/or chemotherapies. In some embodiments, the present disclosure provides a combination therapy, which comprises an anti-cancer therapy described herein (e.g., an anti-CD137 antibody, a checkpoint blockade immunotherapy) in combination with one or more additional therapies or therapeutic agents for separate, sequential or simultaneous administration. The term “additional therapy” or “additional therapeutic agent” may refer to a therapy or therapeutic agent which does not employ the same immunotherapy as is provided in the anti-cancer therapy. In some embodiments, the present disclosure provides a combination therapy for treating cancer in a mammal, which comprises administering to the mammal an effective amount of an anti-cancer therapy of the present disclosure (e.g., an anti-CD137 antibody, a checkpoint blockade immunotherapy) in combination with one or more additional therapeutic agents.

A wide variety of cancer therapeutic agents may be used in combination with a binding molecule provided by the present disclosure. One of ordinary skill in the art will recognize the presence and development of other cancer therapies which can be used in combination with the methods and binding molecules of the present disclosure, and will not be restricted to those forms of therapy set forth herein. Examples of categories of additional therapeutic agents that may be used in the combination therapy for treating cancer include (1) chemotherapeutic agents, (2) immunotherapeutic agents, and (3) hormone therapeutic agents.

The term “chemotherapeutic agent” refers to a chemical or biological substance that can cause death of cancer cells, or interfere with growth, division, repair, and/or function of cancer cells. Examples of chemotherapeutic agents include those that are disclosed in WO 2006/129163, and US 20060153808, the disclosures of which are incorporated herein by reference. Examples of particular chemotherapeutic agents include: (1) alkylating agents, such as chlorambucil (LEUKERAN), mcyclophosphamide (CYTOXAN), ifosfamide (IFEX), mechlorethamine hydrochloride (MUSTARGEN), thiotepa (THIOPLEX), streptozotocin (ZANOSAR), carmustine (BICNU, GLIADEL WAFER), lomustine (CEENU), and dacarbazine (DTIC-DOME); (2) alkaloids or plant vinca alkaloids, including cytotoxic antibiotics, such as doxorubicin (ADRIAMYCIN), epirubicin (ELLENCE, PHARMORUBICIN), daunorubicin (CERUBIDINE, DAUNOXOME), nemorubicin, idarubicin (IDAMYCIN PFS, ZAVEDOS), mitoxantrone (DHAD, NOVANTRONE). dactinomycin (actinomycin D, COSMEGEN), plicamycin (MITHRACIN), mitomycin (MUTAMYCIN), and bleomycin (BLENOXANE), vinorelbine tartrate (NAVELBINE)), vinblastine (VELBAN), vincristine (ONCOVIN), and vindesine (ELDISINE); (3) antimetabolites, such as capecitabine (XELODA), cytarabine (CYTOSAR-U), fludarabine (FLUDARA), gemcitabine (GEMZAR), hydroxyurea (HYDRA), methotrexate (FOLEX, MEXATE, TREXALL), nelarabine (ARRANON), trimetrexate (NEUTREXIN), and pemetrexed (ALIMTA); (4) Pyrimidine antagonists, such as 5-fluorouracil (5-FU); capecitabine (XELODA), raltitrexed (TOMUDEX), tegafur-uracil (UFTORAL), and gemcitabine (GEMZAR); (5) taxanes, such as docetaxel (TAXOTERE), paclitaxel (TAXOL); (6) platinum drugs, such as cisplatin (PLATINOL) and carboplatin (PARAPLATIN), and oxaliplatin (ELOXATIN); (7) topoisomerase inhibitors, such as irinotecan (CAMPTOSAR), topotecan (HYCAMTIN), etoposide (ETOPOPHOS, VEPESSID, TOPOSAR), and teniposide (VUMON); (8) epipodophyllotoxins (podophyllotoxin derivatives), such as etoposide (ETOPOPHOS, VEPESSID, TOPOSAR); (9) folic acid derivatives, such as leucovorin (WELLCOVORIN); (10) nitrosoureas, such as carmustine (BiCNU), lomustine (CeeNU); (11) inhibitors of receptor tyrosine kinase, including epidermal growth factor receptor (EGFR), vascular endothelial growth factor (VEGF), insulin receptor, insulin-like growth factor receptor (IGFR), hepatocyte growth factor receptor (HGFR), and platelet-derived growth factor receptor (PDGFR), such as gefitinib (IRESSA), erlotinib (TARCEVA), bortezomib (VELCADE), imatinib mesylate (GLEEVEC), genefitinib, lapatinib, sorafenib, thalidomide, sunitinib (SUTENT), axitinib, rituximab (RITUXAN, MABTHERA), trastuzumab (HERCEPTIN), cetuximab (ERBITUX), bevacizumab (AVASTIN), and ranibizumab (LUCENTIS), lym-1 (ONCOLYM), antibodies to insulin-like growth factor-1 receptor (IGF-1R) that are disclosed in WO2002/053596); (12) angiogenesis inhibitors, such as bevacizumab (AVASTIN), suramin (GERMANIN), angiostatin, SU5416, thalidomide, and matrix metalloproteinase inhibitors (such as batimastat and marimastat), and those that are disclosed in WO2002055106; and (13) proteasome inhibitors, such as bortezomib (VELCADE).

The term “immunotherapeutic agents” refers to a chemical or biological substance that can enhance an immune response of a mammal. Examples of immunotherapeutic agents include: bacillus Calmette-Guerin (BCG); cytokines such as interferons; vaccines such as MyVax personalized immunotherapy, Onyvax-P, Oncophage, GRNVACI, Favld, Provenge, GVAX, Lovaxin C, BiovaxID, GMXX, and NeuVax; and antibodies such as alemtuzumab (CAMPATH), bevacizumab (AVASTIN), cetuximab (ERBITUX), gemtuzunab ozogamicin (MYLOTARG), ibritumomab tiuxetan (ZEVALIN), panitumumab (VECTIBIX), rituximab (RITUXAN, MABTHERA), trastuzumab (HERCEPTIN), tositumomab (BEXXAR), ipilimumab (YERVOY) tremelimumab, CAT-3888, agonist antibodies to OX40 receptor (such as those disclosed in WO2009/079335), agonist antibodies to CD40 receptor (such as those disclosed in WO2003/040170, and TLR-9 agonists (such as those disclosed in WO2003/015711, WO2004/016805, and WO2009/022215).

The term “hormone therapeutic agent” refers to a chemical or biological substance that inhibits or eliminates the production of a hormone, or inhibits or counteracts the effect of a hormone on the growth and/or survival of cancerous cells. Examples of such agents suitable for the methods herein include those that are disclosed in US20070117809. Examples of particular hormone therapeutic agents include tamoxifen (NOLVADEX), toremifene (Fareston), fulvestrant (FASLODEX), anastrozole (ARIMIDEX), exemestane (AROMASIN), letrozole (FEMARA), megestrol acetate (MEGACE), goserelin (ZOLADEX), and leuprolide (LUPRON). The binding molecules of this disclosure may also be used in combination with non-drug hormone therapies such as (1) surgical methods that remove all or part of the organs or glands which participate in the production of the hormone, such as the ovaries, the testicles, the adrenal gland, and the pituitary gland, and (2) radiation treatment, in which the organs or glands of the patient are subjected to radiation in an amount sufficient to inhibit or eliminate the production of the targeted hormone.

The combination therapy for treating cancer also encompasses the combination of a binding molecule with surgery to remove a tumor. The binding molecule may be administered to the mammal before, during, or after the surgery.

The combination therapy for treating cancer also encompasses combination of a binding molecule with radiation therapy, such as ionizing (electromagnetic) radiotherapy (e.g., X-rays or gamma rays) and particle beam radiation therapy (e.g., high linear energy radiation). The source of radiation can be external or internal to the mammal. The binding molecule may be administered to the mammal before, during, or after the radiation therapy.

Administering Immunotherapies

In some embodiments, the present disclosure relates to the administration of an effective amount of an anti-cancer therapy (e.g., an anti-CD137 antibody, a checkpoint blockade immunotherapy). In some embodiments, the anti-cancer therapy (e.g., an anti-CD137 antibody, a checkpoint blockade immunotherapy) is used to treat or delay progression of cancer in a subject. In some embodiments, the anti-cancer therapy (e.g., an anti-CD137 antibody, a checkpoint blockade immunotherapy) delays the onset of cancer, including biochemical, histological and/or behavioral symptoms of cancer, its complications and intermediate pathological phenotypes presenting during development of cancer. In some embodiments, the anti-cancer therapy (e.g., an anti-CD137 antibody, a checkpoint blockade immunotherapy) delays development of cancer and/or slows the progression of cancer and/or prolongs survival of the subject.

In some embodiments, the anti-cancer therapy (e.g., an anti-CD137 antibody, a checkpoint blockade immunotherapy) is capable of inhibiting tumor cell growth and/or proliferation. In some embodiments, the tumor cell growth and/or proliferation is inhibited by at least about 5% when contacted with the anti-cancer therapy (e.g., an anti-CD137 antibody, a checkpoint blockade immunotherapy) relative to corresponding tumor cells not contacted with the anti-cancer therapy. For example, the tumor cell growth and/or proliferation is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% when contacted with the anti-cancer therapy (e.g., an anti-CD137 antibody, a checkpoint blockade immunotherapy) relative to corresponding tumor cells not contacted with the anti-cancer therapy. In some embodiments, the tumor cell growth and/or proliferation is inhibited by at least about 1-fold when contacted with the anti-cancer therapy (e.g., an anti-CD137 antibody, a checkpoint blockade immunotherapy) relative to corresponding tumor cells not contacted with the anti-cancer therapy. For example, the tumor cell growth and/or proliferation is inhibited by at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5 fold, at least about 8-fold, at least about 8.5-fold, at least about 9-fold, at least about 9.5-fold, at least about 10-fold, at least about 100-fold, or at least about 1000-fold when contacted with the anti-cancer therapy (e.g., an anti-CD137 antibody, a checkpoint blockade immunotherapy) relative to corresponding tumor cells not contacted with the anti-cancer therapy.

In some embodiments, the anti-cancer therapy (e.g., an anti-CD137 antibody, a checkpoint blockade immunotherapy) is capable of reducing tumor volume in a subject when the subject is administered the anti-cancer therapy. In some embodiments, the anti-cancer therapy (e.g., an anti-CD137 antibody, a checkpoint blockade immunotherapy) is capable of reducing tumor volume in a subject by at least about 5% relative to the initial tumor volume in the subject (e.g., prior to administration of the anti-cancer therapy). For example, the anti-cancer therapy (e.g., an anti-CD137 antibody, a checkpoint blockade immunotherapy) is capable of reducing tumor volume in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% relative to the initial tumor volume in the subject (e.g., prior to administration of the anti-cancer therapy). In some embodiments, the anti-cancer therapy (e.g., an anti-CD137 antibody, a checkpoint blockade immunotherapy) is capable of reducing tumor volume in a subject by at least about 1-fold relative to the initial tumor volume in the subject (e.g., prior to administration of the anti-cancer therapy). For example, the anti-cancer therapy (e.g., an anti-CD137 antibody, a checkpoint blockade immunotherapy) is capable of reducing tumor volume in a subject by at least about 1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5 fold, at least about 8-fold, at least about 8.5-fold, at least about 9-fold, at least about 9.5-fold, at least about 10-fold, at least about 100-fold, or at least about 1000-fold relative to the initial tumor volume in the subject (e.g., prior to administration of the anti-cancer therapy). Methods of monitoring tumor cell growth and/or proliferation, tumor volume, and/or tumor inhibition are known in the art, including, for example, via the methods described in Example 3 below.

In some embodiments, the anti-cancer therapy (e.g., an anti-CD137 antibody, a checkpoint blockade immunotherapy) has therapeutic effect on a cancer. In some embodiments, the anti-cancer therapy (e.g., an anti-CD137 antibody, a checkpoint blockade immunotherapy) reduces one or more signs or symptoms of a cancer. In some embodiments, a subject suffering from a cancer goes into partial or complete remission when administered the anti-cancer therapy (e.g., an anti-CD137 antibody, a checkpoint blockade immunotherapy).

Binding molecules and pharmaceutical compositions of the present disclosure are useful for therapeutic, diagnostic, or other purposes, such as modulating an immune response, treating cancer, enhancing efficacy of other cancer therapy, enhancing vaccine efficacy, or treating autoimmune diseases. In some embodiments, the present disclosure provides methods of treating a disorder in a mammal (e.g., after measuring CD137L expression in a sample taken from the mammal), which comprises administering to the mammal in need of treatment an effective amount of an anti-cancer therapy described herein.

The anti-cancer therapies of the present disclosure (e.g., an anti-CD137 antibody, a checkpoint blockade immunotherapy) may be administered via any suitable enteral or parenteral route of administration. The term “enteral route” of administration may refer to the administration via any part of the gastrointestinal tract. Enteral routes of administration include, for example, oral, mucosal, buccal, rectal, intragastric, etc. The term “Parenteral route” of administration may refer to a route of administration other than enteral route. Parenteral routes of administration include, for example, intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, topical administration, etc. The anti-cancer therapies of the disclosure (e.g., an anti-CD137 antibody, a checkpoint blockade immunotherapy) may be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art

An effective amount of an anti-cancer therapy of the present disclosure (e.g., an anti-CD137 antibody, a checkpoint blockade immunotherapy) may range from about 0.001 to about 500 mg/kg, including, for example, about 0.01 to about 100 mg/kg, of the body weight of the subject. For example, the amount may be about 0.3 mg/kg, 1 mg/kg, 3 mg/kg, 5 mg/kg, 10 mg/kg, 50 mg/kg, or 100 mg/kg of body weight of the subject. In some embodiments, the effective amount of the anti-cancer therapy (e.g., an anti-CD137 antibody, a checkpoint blockade immunotherapy) is in the range of about 0.01-30 mg/kg of body weight of the subject. In some other embodiments, the effective amount of the anti-cancer therapy (e.g., an anti-CD137 antibody, a checkpoint blockade immunotherapy) is in the range of about 0.05-15 mg/kg of body weight of the subject. The precise dosage level to be administered can be readily determined by a person skilled in the art and will depend on a number of factors, such as the type, and severity of the disorder to be treated, the particular anti-cancer therapy employed, the route of administration, the time of administration, the duration of the treatment, the particular additional therapy employed, the age, sex, weight, condition, general health and prior medical history of the patient being treated, and like factors well known in the medical arts.

An anti-cancer therapy of the present disclosure (e.g., an anti-CD137 antibody, a checkpoint blockade immunotherapy) may be administered on multiple occasions. Intervals between single doses can be, for example, daily, weekly, monthly, every three months or yearly. An exemplary treatment regimen entails administration once per week, once every two weeks, once every three weeks, once every four weeks, once a month, once every three months or once every three to six months. The precise timing of dosages to be administered can be readily determined by a person skilled in the art.

The present disclosure will be more fully understood by reference to the following examples. The examples should not, however, be construed as limiting the scope of the present disclosure. It is understood that the examples and embodiments described herein are for illustrative purposes only, and that various modifications or changes in light thereof will be suggested to persons skilled in the art, and are to be included within the spirit and purview of this application and scope of the appended claims.

### EXAMPLES

### Example 1: Identification of Antibodies Recognizing the Intracellular Fragment of Human CD137L

To identify antibodies that specifically bind the intracellular or membrane bound human CD137L, but not the shedded CD137L ECD, the intracellular fragment of human CD137L peptide 01 (MEYASDASLDPEAPWPPAPRARACRVLP; SEQ ID NO:1) and peptide 02 (MEYASDASLDPEAPWPPAPRARA; SEQ ID NO:2) were synthesized as biotinylated peptides and used as antigens for antibody discovery through phagemid libraries. The CD137L peptide 02 is identical to peptide 01 except that it lacks the C-terminal 5 residues.

A total of three rounds of panning were conducted. After the final round of panning, single-colony supernatant ELISA was performed to identify the primary hits that specifically recognize peptide 01 or peptide 02. The primary hits were defined as those whose ELISA signals were at least twice that of background. The plasmids were then extracted, the DNA fragments encoding the light chain and heavy chain were sequenced, and the unique clones were expressed and purified for further confirmation through ELISA or ForteBio. The confirmed hits were then converted into IgGs with Fc from mouse IgG2a expressed in mammalian cells and purified through protein A affinity column. The affinity and specificity of the purified IgGs were examined through ELISA with the CD137L peptide 01 and peptide 02, as well as four peptides derived from other proteins as negative controls.

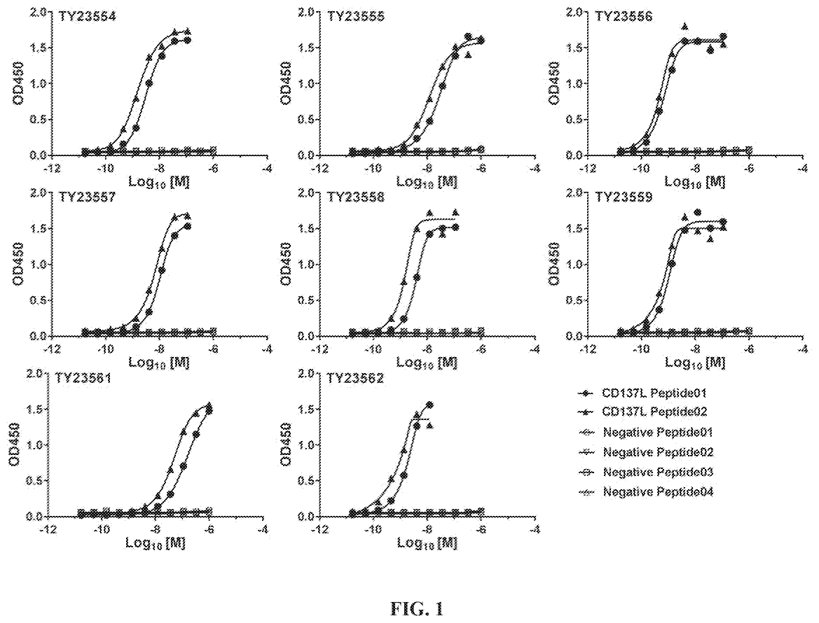

As shown in FIG. 1, all eight IgGs demonstrated high specificity against the CD137L peptide 01 and peptide 02, with minimal reactivity against all four negative control peptides. The measured EC50 ranges from sub-nanomolar (0.66 nM) for TY23556 to 166.5 nM for TY23561 (Table A).

TABLE A
Measured EC50 (nM) antibodies against CD137L peptide 01 and peptide 02.

| | TY23554 | TY23555 | TY23556 | TY23557 | TV23558 | TY23559 | TY23561 | TY23562 |
|---|---|---|---|---|---|---|---|---|
| Peptide | 3.13 | 28.52 | 0.66 | 10.11 | 3.84 | 1.03 | 166.5 | 2.04 |
| 01 | | | | | | | | |
| Peptide | 1.59 | 12.30 | 0.48 | 6.77 | 1.51 | 0.65 | 50.01 | 0.74 |
| 02 | | | | | | | | |

### Example 2: Identification of Antibodies Recognizing Intracellular or Membrane Bound Human CD137L in Fixed Mammalian Cells

To identify antibodies that recognize intracellular or membrane bound human CD137L in fixed mammalian cells, a CHO-S cell derived mammalian stable cell line (CHO-S-CD137L) was created in house as a positive control, it has high expression of full length human CD137L. CHO-S-CD137L and the negative control CHO-S cells were washed, fixed and permeabilized in a similar fashion as in immunohistochemistry, followed by analysis through Beckman CytoFlex. All eight IgGs, as well as a negative isotype control IgG, were then screened at a single concentration.

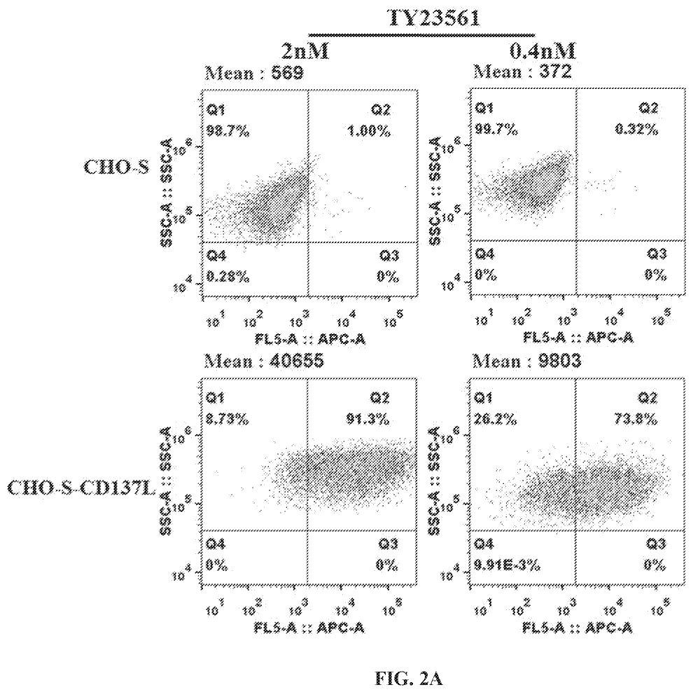

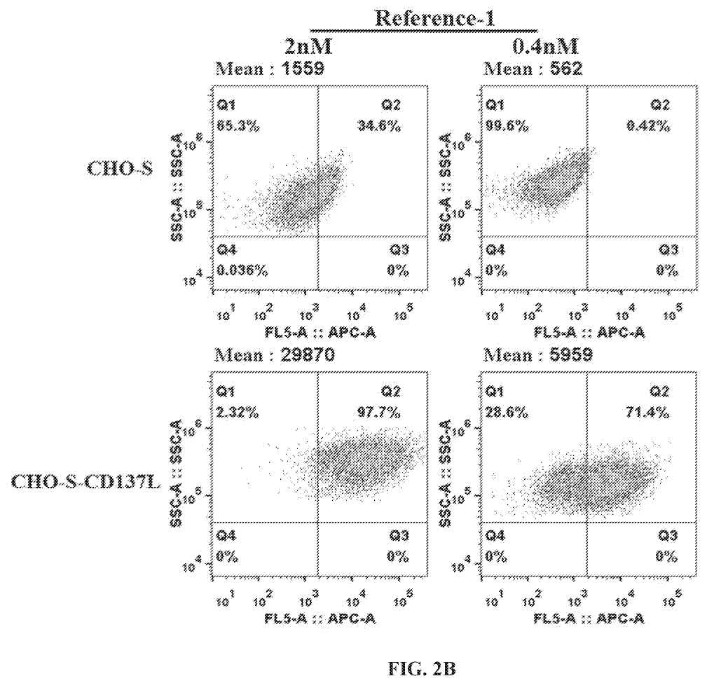

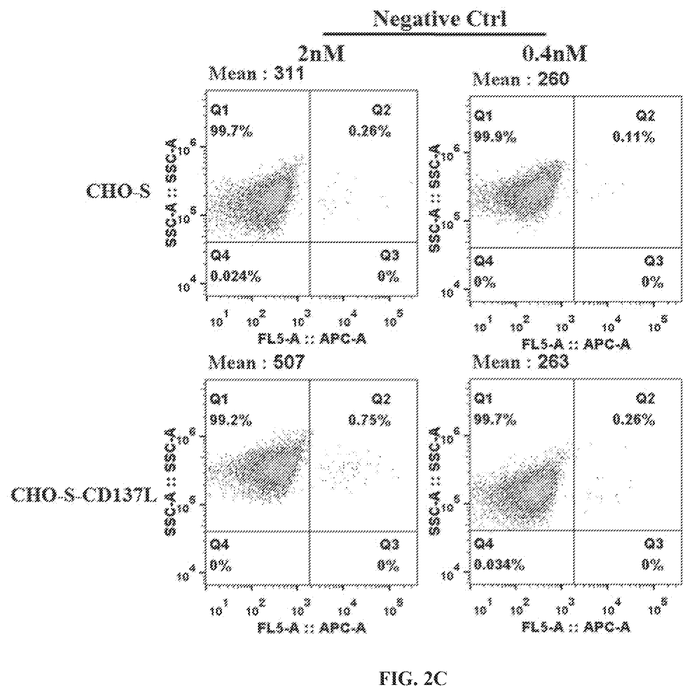

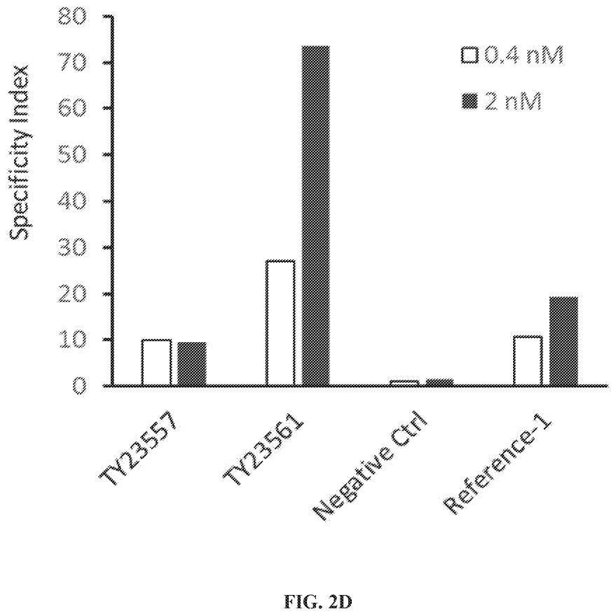

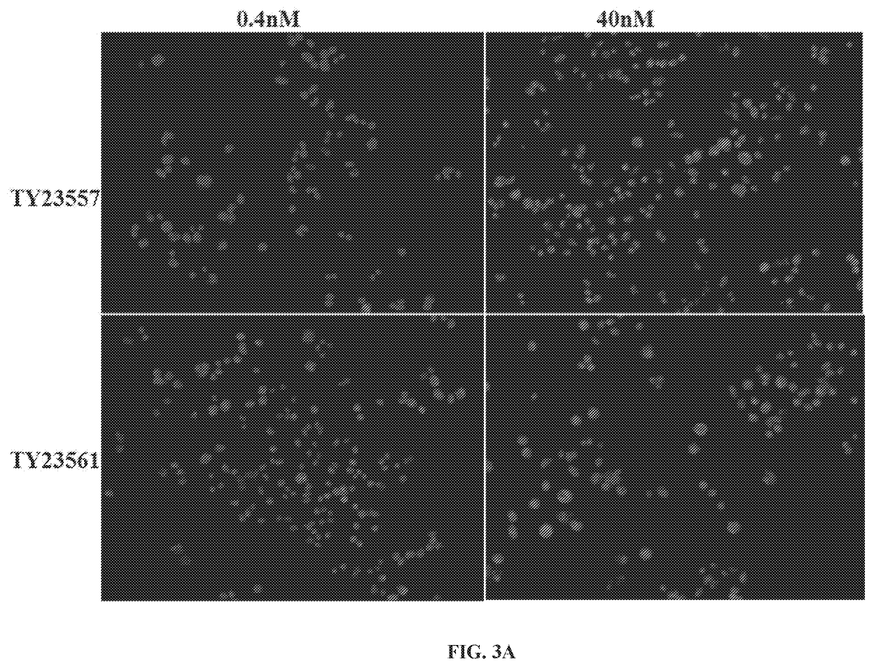

Interestingly, it was found that TY23561 displayed the highest specificity for CHO-S-CD137L. To optimize the concentration of TY23561 and compare its performance with a commercial antibody (Reference-1)) for IHC application, several concentrations of antibodies were then tested. While the negative isotype control antibody did not stain either CHO-S-CD137L or CHO-S cells (FIG. 2C), both TY23561 (FIG. 2A) and the Reference-1 (FIG. 2B) demonstrated good specificity toward CD137L present in CHO-S-CD137L. Importantly, TY23561 demonstrated higher staining of CHO-S-CD137L cells than the Reference-1, while at the same time lower background staining of CHO-S cells at all concentrations tested. If a specificity index is operationally defined as the ratio of MFI (CHO-S-CD137L) over MFI (CHO-S), clearly TY23561 demonstrated much higher specificity than the Reference-1 antibody at all concentrations tested. For example, at 0.4 nM, the specificity index was 27 for TY23561, while it was 10.7 for Reference-1; while at 2 nM, the specificity index was 73.5 for TY23561, it was 19.4 for Reference-1 (FIG. 2D). Therefore, TY23561 may be a much more specific antibody for IHC applications than the commercial antibody.

### Example 3: Analysis of Antibody Specificity Through Immunofluorescence Analysis

The specificity of the antibodies were further confirmed with immunofluorescence analysis. To that end, CHO-S and CHO-S-CD137L cells were cultured on cover slides overnight. Cells were washed with DPBS followed by fixing and permeabilizing with 2% (v/v) paraformaldehyde and 0.1% Triton X 100. Then the cells were incubated with TY23557, TY23561, negative isotype control and Reference-1 (positive control) for one hour at room temperature. After washing by 0.05% PBST, PE Goat anti-mouse IgG Fc were added and staining for one hour at room temperature. The samples were analyzed with fluorescent microscopy.

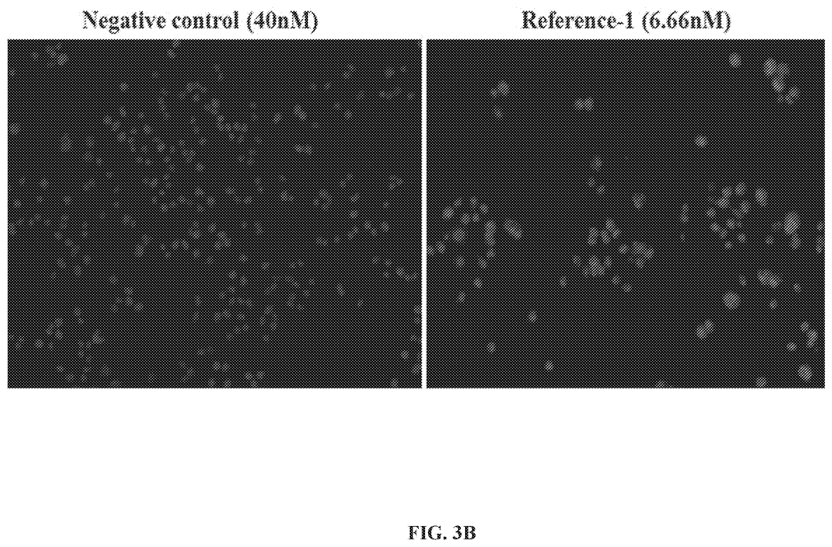

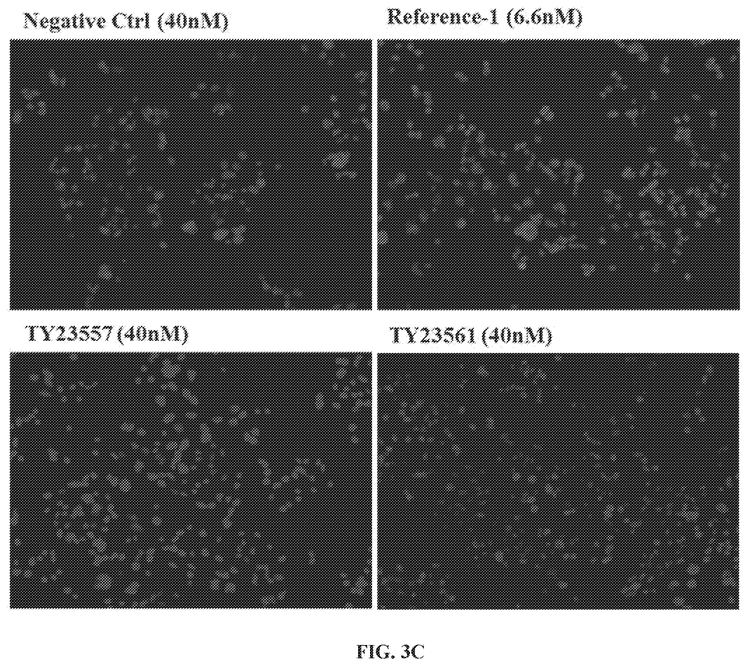

As shown in FIGS. 3A & 3B, both TY23557 and TY23561 showed positive staining on CHO-S-CD137L cells with negative staining on CHO-S cells, compared to negative control and Reference-1 staining. The staining signal of TY23561 was higher than TY23557. Reference-1 also stains strongly on CHO-S-CD137L cells, but also showed non-specific staining on some CHO-S cells. These results suggest that anti-CD137L antibody TY23561 performed the best among these anti-CD137L antibodies tested in immunofluorescence staining conditions.

### Example 4: Specificity of Immunohistochemical (IHC) Staining Using Anti-hCD137L Antibodies

Methods

For cell line staining, parental and human CD137 ligand-transfected CHO-S cells were FFPE processed and stained with different anti-CD137L antibodies: Reference-1 (ThermoFisher, 14-9056-82), Reference-2 (Abcam, ab223160), TY23557 and TY23561, followed by an HRP-labeled 2^{nd} anti-mouse IgG detection and 3,3′-diaminobenzidine (DAB) chromogenic reaction. The cell nuclei were counterstained with Hematoxylin.

Human tonsil is a lymphoid organ, which contains lymphocytes expressing CD137L and serves as a CD137L-positive sample. For staining of human tonsil tissue, human tonsil sections were FFPE processed and stained with anti-CD137L antibodies Reference-1, Reference-2, TY23557 and TY23561, followed by an HRP-labeled 2^{nd} anti-mouse IgG detection and DAB chromogenic reaction. The cell nuclei were counterstained with Hematoxylin.

Results

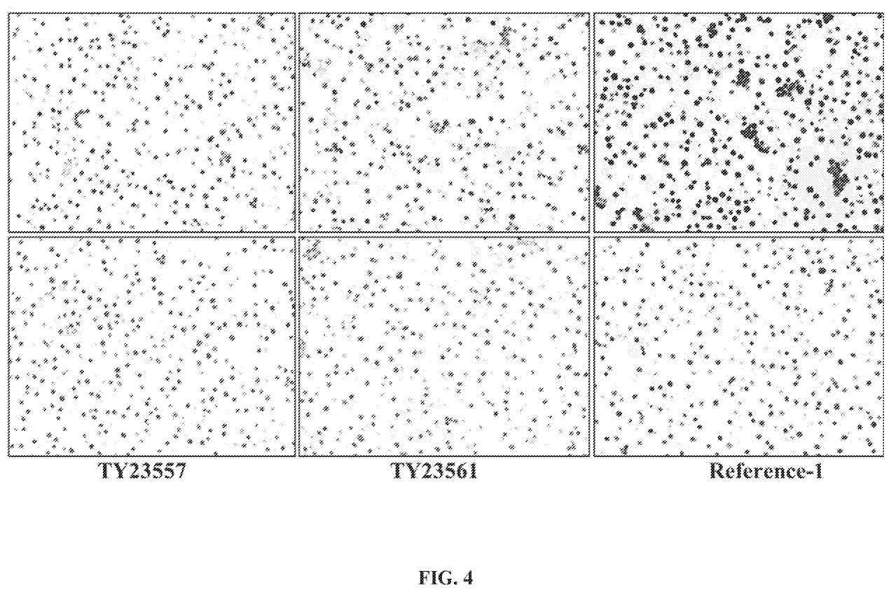

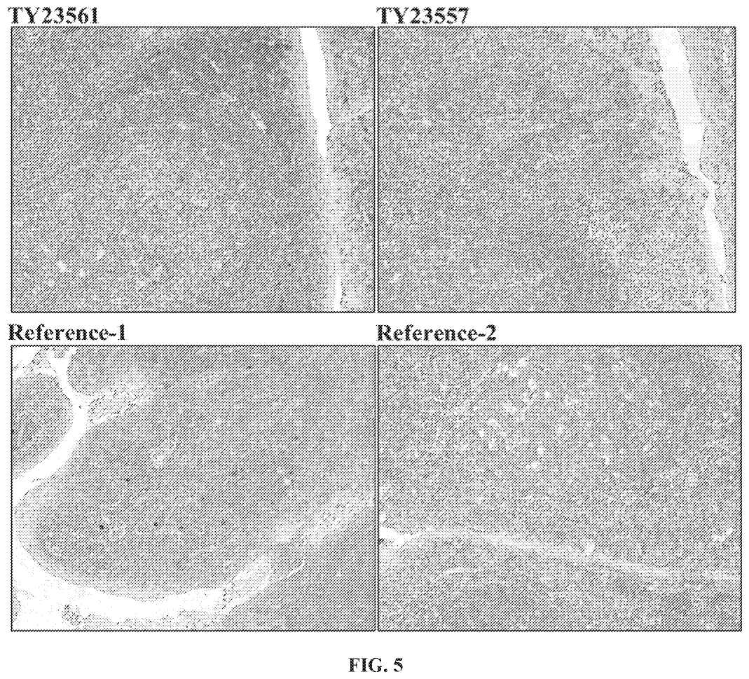

All anti-CD137L antibodies stained CHO-S cells stably transfected with the full-length CD137L gene expression cassette (brown staining). In the parental CHO-S cells negative for CD137L expression, Reference-1 stained weakly, and other tested antibodies showed no staining (FIG. 4). These results indicate that the anti-CD137L antibodies recognized CD137L expressed by CHO-S cells under IHC staining conditions with relative specificity.

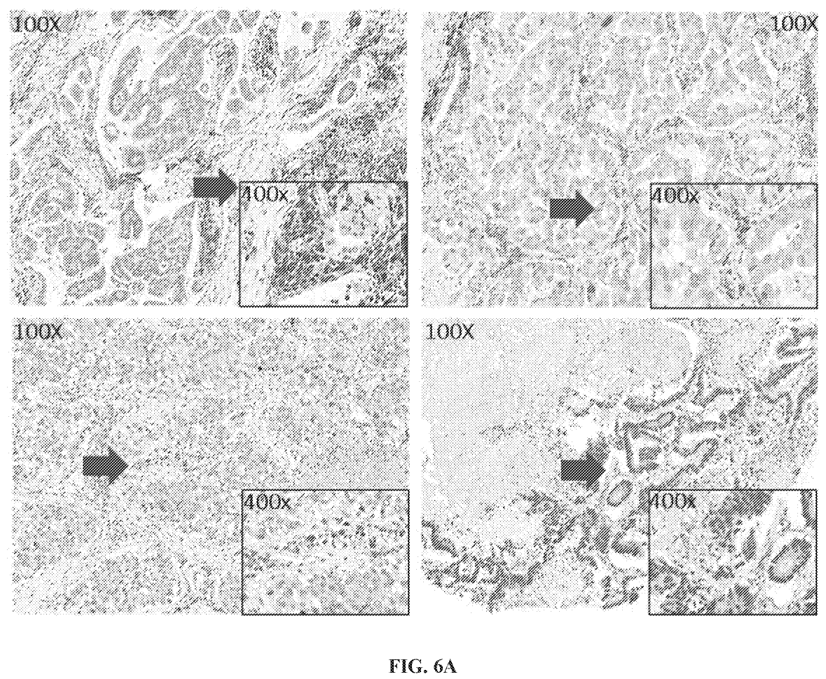

Using human tonsil tissue (FIG. 5), no specific staining was observed with Abcam anti-CD137L antibody, whereas the other 3 antibodies showed positive staining in certain cells in human tonsil tissue (brown staining). However, among these 3 antibodies, ThermoFisher anti-CD137 and TY23557 showed significant background staining, whereas TY23561 showed much better staining specificity.

### Example 5: IHC Staining of CD137L in Patient Samples Using Anti-CD137L Antibody TY23561

Methods

FFPE tumor sections from different patients with lung cancers or lymphoma were commercially obtained and stained with TY23561 and an HRP-labeled 2^{nd} anti-mouse IgG, followed by DAB chromogenic reaction. An automated Leica Bond-RX immunostainer was used at ER1 retrieval setting, and staining was optimized using appropriate FFPE controls. The stained sections were scanned with a 3D HISTECH Pannoramic MIDI. The cell nuclei were counterstained with Hematoxylin.

Results

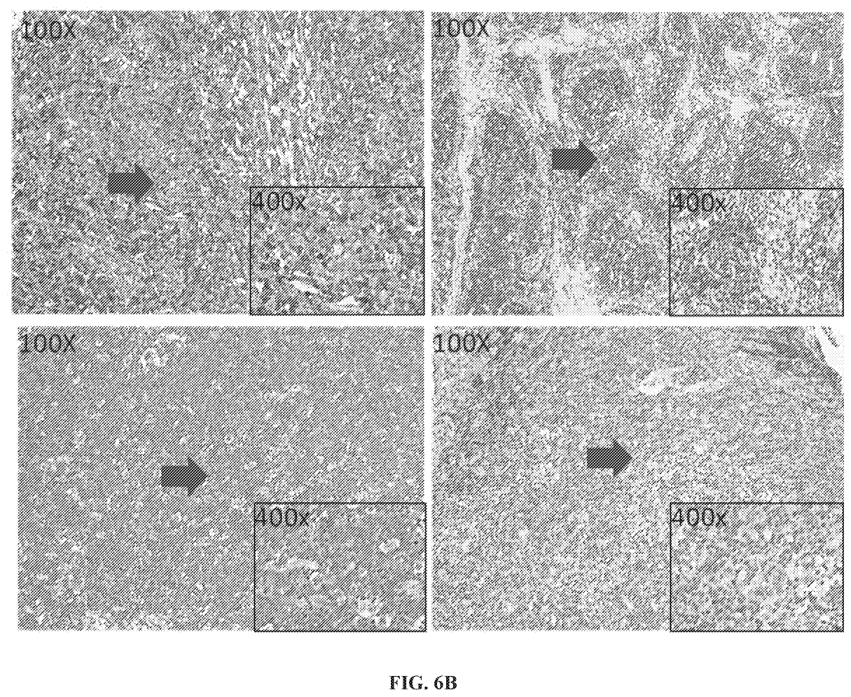

FIGS. 6A & 6B show staining results in tumor sections from patients with lung cancer (FIG. 6A) or lymphoma (FIG. 6B). One representative image is shown for each patient. The results indicate that TY23561 detected varying levels of CD137 ligand expression in tumors from different patients, and the signals (brown staining) ranged from strong, mild, weak, and negative, with signals either in the interstitial cells, tumor cells, or both. These data indicate that anti-CD137L antibody TY23561 was useful in detecting and scoring varying levels of CD137L specifically in patient samples under IHC conditions.

### Example 6: Affinity Maturation of Anti-CD137L Antibodies

Maturation libraries were designed based on the identified candidate antibodies, and variants were introduced into all 6 hyper-variable regions (HVRs), while the framework regions remained unchanged. The maturation libraries were panned against reduced concentrations of CD137L peptide 01 (SEQ ID NO:1), compared with those in Example 1, and the washing conditions were more stringent as well. A total of 48 hits with unique sequences were identified and further characterized (Table B).

TABLE B
Sequences identified during affinity maturation.

| | VH | VL |
|---|---|---|
| Fab ID | SEQID | SEQID |
| Fab8947 | 329 | 330 |
| Fab8948 | 331 | 332 |
| Fab8949 | 241 | 242 |
| Fab8950 | 243 | 244 |
| Fab8951 | 333 | 334 |
| Fab8952 | 335 | 336 |
| Fab8954 | 245 | 246 |
| Fab8963 | 249 | 250 |
| Fab8964 | 251 | 252 |
| Fab8966 | 253 | 254 |
| Fab8969 | 255 | 256 |
| Fab8971 | 257 | 258 |
| Fab8974 | 259 | 260 |
| Fab8976 | 261 | 262 |
| Fab8985 | 263 | 264 |
| Fab8986 | 263 | 264 |
| Fab9010 | 269 | 270 |
| Fab9011 | 271 | 272 |
| Fab9012 | 273 | 274 |
| Fab9013 | 275 | 276 |
| Fab9014 | 277 | 278 |
| Fab9015 | 279 | 280 |
| Fab8955 | 323 | 324 |
| Fab8959 | 247 | 248 |
| Fab8957 | 325 | 326 |
| Fab8958 | 327 | 328 |
| Fab8996 | 267 | 268 |
| Fab9016 | 281 | 282 |
| Fab9017 | 283 | 284 |
| Fab9018 | 285 | 286 |
| Fab9019 | 287 | 288 |
| Fab9020 | 289 | 290 |
| Fab9022 | 291 | 292 |
| Fab9023 | 293 | 294 |
| Fab9024 | 295 | 296 |
| Fab9025 | 297 | 298 |
| Fab9026 | 299 | 300 |
| Fab9027 | 301 | 302 |
| Fab9028 | 303 | 304 |
| Fab9029 | 305 | 306 |
| Fab9030 | 307 | 308 |
| Fab9031 | 309 | 310 |
| Fab9032 | 311 | 312 |
| Fab9033 | 313 | 314 |
| Fab9036 | 315 | 316 |
| Fab9037 | 317 | 318 |
| Fab9038 | 319 | 320 |
| Fab9039 | 321 | 322 |

What is claimed is:

1. An isolated antibody, or antigen-binding fragment thereof, that binds to an intracellular or transmembrane region of human CD137 ligand (CD137L), wherein the antibody or antigen-binding fragment binds to a peptide comprising the amino acid sequence of MEYASDASLDPEAPWPPAPRARACRVLP (SEQ ID NO:1) and/or binds to a peptide comprising the amino acid sequence of MEYASDASLDPEAPWPPAPRARA (SEQ ID NO:2).

2. The antibody or antigen-binding fragment of claim 1, wherein the peptide comprising the amino acid sequence of MEYASDASLDPEAPWPPAPRARACRVLP (SEQ ID NO:1) and/or the peptide comprising the amino acid sequence of MEYASDASLDPEAPWPPAPRARA (SEQ ID NO:2) is less than 50 amino acids in length.

3. The antibody or antigen-binding fragment of claim 1 or claim 2, wherein the peptide comprising the amino acid sequence of MEYASDASLDPEAPWPPAPRARACRVLP (SEQ ID NO:1) and/or the peptide comprising the amino acid sequence of MEYASDASLDPEAPWPPAPRARA (SEQ ID NO:2) does not comprise the extracellular domain of CD137L.

4. The antibody or antigen-binding fragment of any one of claims 1-3, wherein the antibody or antigen-binding fragment binds to a Chinese hamster ovary (CHO) cell that expresses human CD137L.

5. The antibody or antigen-binding fragment of any one of claims 1-4, wherein the antibody or antigen-binding fragment binds to human CD137L in a fixed human tissue sample.

6. The antibody or antigen-binding fragment of claim 5, wherein the fixed human tissue sample is a formalin-fixed paraffin-embedded (FFPE) sample.

7. The antibody or antigen-binding fragment of claim 5 or claim 6, wherein the sample is from human tonsil tissue.

8. The antibody or antigen-binding fragment of claim 5 or claim 6, wherein the sample is from human tumor tissue.

9. The antibody or antigen-binding fragment of any one of claims 1-8, wherein the antibody comprises a heavy chain variable region and a light chain variable region, wherein:
(a) the heavy chain variable region comprises an HVR-H1 comprising the amino acid sequence of SEQ ID NO:4, an HVR-H2 comprising the amino acid sequence of SEQ ID NO:5, and an HVR-H3 comprising the amino acid sequence of SEQ ID NO:6, and/or the light chain variable region comprises an HVR-L1 comprising the amino acid sequence of SEQ ID NO:7, an HVR-L2 comprising the amino acid sequence of SEQ ID NO:8, and an HVR-L3 comprising the amino acid sequence of SEQ ID NO:9;
(b) the heavy chain variable region comprises an HVR-H1 comprising the amino acid sequence of SEQ ID NO:10, an HVR-H2 comprising the amino acid sequence of SEQ ID NO:11, and an HVR-H3 comprising the amino acid sequence of SEQ ID NO:12, and/or the light chain variable region comprises an HVR-L1 comprising the amino acid sequence of SEQ ID NO:13, an HVR-L2 comprising the amino acid sequence of SEQ ID NO:14, and an HVR-L3 comprising the amino acid sequence of SEQ ID NO:15;
(c) the heavy chain variable region comprises an HVR-H1 comprising the amino acid sequence of SEQ ID NO:16, an HVR-H2 comprising the amino acid sequence of SEQ ID NO:17, and an HVR-H3 comprising the amino acid sequence of SEQ ID NO:18, and/or the light chain variable region comprises an HVR-L1 comprising the amino acid sequence of SEQ ID NO:19, an HVR-L2 comprising the amino acid sequence of SEQ ID NO:20, and an HVR-L3 comprising the amino acid sequence of SEQ ID NO:21;
(d) the heavy chain variable region comprises an HVR-H1 comprising the amino acid sequence of SEQ ID NO:22, an HVR-H2 comprising the amino acid sequence of SEQ ID NO:23, and an HVR-H3 comprising the amino acid sequence of SEQ ID NO:24, and/or the light chain variable region comprises an HVR-L1 comprising the amino acid sequence of SEQ ID NO:25, an HVR-L2 comprising the amino acid sequence of SEQ ID NO:26, and an HVR-L3 comprising the amino acid sequence of SEQ ID NO:27;
(e) the heavy chain variable region comprises an HVR-H1 comprising the amino acid sequence of SEQ ID NO:28, an HVR-H2 comprising the amino acid sequence of SEQ ID NO:29, and an HVR-H3 comprising the amino acid sequence of SEQ ID NO:30, and/or the light chain variable region comprises an HVR-L1 comprising the amino acid sequence of SEQ ID NO:31, an HVR-L2 comprising the amino acid sequence of SEQ ID NO:32, and an HVR-L3 comprising the amino acid sequence of SEQ ID NO:33;
(f) the heavy chain variable region comprises an HVR-H1 comprising the amino acid sequence of SEQ ID NO:34, an HVR-H2 comprising the amino acid sequence of SEQ ID NO:35, and an HVR-H3 comprising the amino acid sequence of SEQ ID NO:36, and/or the light chain variable region comprises an HVR-L1 comprising the amino acid sequence of SEQ ID NO:37, an HVR-L2 comprising the amino acid sequence of SEQ ID NO:38, and an HVR-L3 comprising the amino acid sequence of SEQ ID NO:39;
(g) the heavy chain variable region comprises an HVR-H1 comprising the amino acid sequence of SEQ ID NO:40, an HVR-H2 comprising the amino acid sequence of SEQ ID NO:41, and an HVR-H3 comprising the amino acid sequence of SEQ ID NO:42, and/or the light chain variable region comprises an HVR-L1 comprising the amino acid sequence of SEQ ID NO:43, an HVR-L2 comprising the amino acid sequence of SEQ ID NO:44, and an HVR-L3 comprising the amino acid sequence of SEQ ID NO:45; or
(h) the heavy chain variable region comprises an HVR-H1 comprising the amino acid sequence of SEQ ID NO:46, an HVR-H2 comprising the amino acid sequence of SEQ ID NO:47, and an HVR-H3 comprising the amino acid sequence of SEQ ID NO:48, and/or the light chain variable region comprises an HVR-L1 comprising the amino acid sequence of SEQ ID NO:49, an HVR-L2 comprising the amino acid sequence of SEQ ID NO:50, and an HVR-L3 comprising the amino acid sequence of SEQ ID NO:51.

10. The antibody or antigen-binding fragment of any one of claims 1-9, wherein:
(a) the heavy chain variable region comprises the amino acid sequence of SEQ ID NO:52, and/or the light chain variable region comprises the amino acid sequence of SEQ ID NO:53;
(b) the heavy chain variable region comprises the amino acid sequence of SEQ ID NO:54, and/or the light chain variable region comprises the amino acid sequence of SEQ ID NO:55;
(c) the heavy chain variable region comprises the amino acid sequence of SEQ ID NO:56, and/or the light chain variable region comprises the amino acid sequence of SEQ ID NO:57;
(d) the heavy chain variable region comprises the amino acid sequence of SEQ ID NO:58, and/or the light chain variable region comprises the amino acid sequence of SEQ ID NO: 59;
(e) the heavy chain variable region comprises the amino acid sequence of SEQ ID NO:60, and/or the light chain variable region comprises the amino acid sequence of SEQ ID NO:61;
(f) the heavy chain variable region comprises the amino acid sequence of SEQ ID NO:62, and/or the light chain variable region comprises the amino acid sequence of SEQ ID NO:63;
(g) the heavy chain variable region comprises the amino acid sequence of SEQ ID NO:64, and/or the light chain variable region comprises the amino acid sequence of SEQ ID NO:65; or
(h) the heavy chain variable region comprises the amino acid sequence of SEQ ID NO:66, and/or the light chain variable region comprises the amino acid sequence of SEQ ID NO:67.

11. The antibody or antigen-binding fragment of any one of claims 1-10, wherein the antibody comprises a mouse IgG2a Fc region.

12. A polynucleotide encoding the antibody or antigen-binding fragment of any one of claims 1-11.

13. A vector comprising the polynucleotide of claim 12.

14. The vector of claim 13, wherein the vector is an expression vector.

15. A host cell comprising the vector of claim 13 or claim 14.

16. A method of making an antibody or antigen-binding fragment, comprising culturing the host cell of claim 15 under conditions suitable for producing the antibody or antigen-binding fragment.

17. The method of claim 16, further comprising recovering the antibody or antigen-binding fragment produced by the cell.

18. A method of detecting a level of expression of human CD137L in a sample, the method comprising:
(a) contacting a human tissue sample with the antibody or antigen-binding fragment of any one of claims 1-11; and
(b) detecting binding of the antibody or antigen-binding fragment to the sample, wherein binding of the antibody or antigen-binding fragment to the sample indicates the level of expression of human CD137L in the sample.

19. The method of claim 18, wherein binding of the antibody or antigen-binding fragment to the sample is detected in (c) by immunohistochemistry (IHC).

20. The method of claim 18 or claim 19, further comprising, prior to (a), obtaining a human tissue sample.

21. A method of treating or delaying progression of cancer in a subject in need thereof, the method comprising:
(a) obtaining a sample from the individual;
(b) measuring level of expression of CD137L in the sample using the antibody of any one of claims 1-11; and
(c) if the level of expression of CD137L in the sample is lower than a reference level, administering an effective amount of an anti-CD137 antibody to the individual.

22. The method of claim 21, wherein the level of expression of CD137L in the sample is measured in (b) using IHC.

23. The method of any one of claims 18-22, wherein the level of expression of CD137L in the sample is below the limit of detection.

24. The method of any one of claims 18-22, wherein the sample is a fixed sample.

25. The method of any one of claims 18-24, wherein the sample is a formalin-fixed paraffin-embedded (FFPE) sample.

26. The method of any one of claims 18-25, wherein the sample is a tumor biopsy sample.

27. The method of any one of claims 18-25, wherein the sample comprises one or more cancer cells.

28. The method of any one of claims 19-25, wherein the sample is a tumor sample from the cancer of the individual.

29. The method of any one of claims 18-28, wherein the level of expression of CD137L is the level of expression of CD137L by cancer cells.

30. The method of any one of claims 19-29, further comprising administering to the subject a therapeutically effective amount of at least one additional therapeutic agent.

31. The method of claim 30, wherein the at least one additional therapeutic agent is selected from the group consisting of viral gene therapy, immune checkpoint inhibitor, target therapy, radiation therapy, and chemotherapy.

32. The method of claim 30 or claim 31, wherein the at least one additional therapeutic agent is selected from the group consisting of pomalyst, revlimid, lenalidomide, pomalidomide, thalidomide, a DNA-alkylating platinum-containing derivative, cisplatin, 5-fluorouracil, cyclophosphamide, an anti-CTLA4 antibody, an anti-PD-1 antibody, an anti-PD-L1 antibody, an anti-CD20 antibody, an anti-CD40 antibody, an anti-DR5 antibody, an anti-CD1d antibody, an anti-TIM3 antibody, an anti-SLAMF7 antibody, an anti-KIR receptor antibody, an anti-OX40 antibody, an anti-HER2 antibody, an anti-ErbB-2 antibody, an anti-EGFR antibody, cetuximab, rituximab, trastuzumab, pembrolizumab, radiotherapy, single dose radiation, fractionated radiation, focal radiation, whole organ radiation, IL-12, IFNα, GM-CSF, a chimeric antigen receptor, adoptively transferred T cells, an anti-cancer vaccine, and an oncolytic virus.


SEQUENCE LISTING (351 sequences)

SEQ ID NO: 1 - protein, 28 aa, Artificial Sequence
  Synthetic Construct
MEYASDASLDPEAPWPPAPRARACRVLP

SEQ ID NO: 2 - protein, 23 aa, Artificial Sequence
  Synthetic Construct
MEYASDASLDPEAPWPPAPRARA

SEQ ID NO: 3 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 4 - protein, 12 aa, Artificial Sequence
  Synthetic Construct
YSITSGYYWAWI

SEQ ID NO: 5 - protein, 21 aa, Artificial Sequence
  Synthetic Construct
IGIINPNRGSTKYAQKFQGRV

SEQ ID NO: 6 - protein, 19 aa, Artificial Sequence
  Synthetic Construct
ARDGWALSGGYYYYYGLDV

SEQ ID NO: 7 - protein, 11 aa, Artificial Sequence
  Synthetic Construct
RASQDISSDLA

SEQ ID NO: 8 - protein, 9 aa, Artificial Sequence
  Synthetic Construct
DASNLETGV

SEQ ID NO: 9 - protein, 11 aa, Artificial Sequence
  Synthetic Construct
YCQQYDAWPST

SEQ ID NO: 10 - protein, 12 aa, Artificial Sequence
  Synthetic Construct
YSISSGHYWAWI

SEQ ID NO: 11 - protein, 21 aa, Artificial Sequence
  Synthetic Construct
VSVISGSGGSTYYADSVKGRF

SEQ ID NO: 12 - protein, 10 aa, Artificial Sequence
  Synthetic Construct
ARYRSGAFDY

SEQ ID NO: 13 - protein, 11 aa, Artificial Sequence
  Synthetic Construct
RASQSISRWLA

SEQ ID NO: 14 - protein, 9 aa, Artificial Sequence
  Synthetic Construct
AASSLQSGV

SEQ ID NO: 15 - protein, 10 aa, Artificial Sequence
  Synthetic Construct
YCQQAYSIWT

SEQ ID NO: 16 - protein, 12 aa, Artificial Sequence
  Synthetic Construct
YSITSGHYWGWI

SEQ ID NO: 17 - protein, 20 aa, Artificial Sequence
  Synthetic Construct
IGEIYHSGSTYYSPSLKSRV

SEQ ID NO: 18 - protein, 9 aa, Artificial Sequence
  Synthetic Construct
TRGRYGLDY

SEQ ID NO: 19 - protein, 10 aa, Artificial Sequence
  Synthetic Construct
SASSSVGYVQ

SEQ ID NO: 20 - protein, 9 aa, Artificial Sequence
  Synthetic Construct
DASNRATGI

SEQ ID NO: 21 - protein, 11 aa, Artificial Sequence
  Synthetic Construct
YCQQYGSAPIT

SEQ ID NO: 22 - protein, 11 aa, Artificial Sequence
  Synthetic Construct
FTFTGYGIHWV

SEQ ID NO: 23 - protein, 21 aa, Artificial Sequence
  Synthetic Construct
VSAISGSGGSTYYADSVKGRF

SEQ ID NO: 24 - protein, 14 aa, Artificial Sequence
  Synthetic Construct
ARGAYYGGSYYFDY

SEQ ID NO: 25 - protein, 12 aa, Artificial Sequence
  Synthetic Construct
RASESITSSYLA

SEQ ID NO: 26 - protein, 9 aa, Artificial Sequence
  Synthetic Construct
DASSLESGV

SEQ ID NO: 27 - protein, 11 aa, Artificial Sequence
  Synthetic Construct
YCFQGSHYPPT

SEQ ID NO: 28 - protein, 12 aa, Artificial Sequence
  Synthetic Construct
YSISSGHYWSWI

SEQ ID NO: 29 - protein, 20 aa, Artificial Sequence
  Synthetic Construct
IGEIYHSGSTYYSPSLKSRV

SEQ ID NO: 30 - protein, 16 aa, Artificial Sequence
  Synthetic Construct
AREGSDSYGGPDPFDI

SEQ ID NO: 31 - protein, 12 aa, Artificial Sequence
  Synthetic Construct
RARSTVSFPYLH

SEQ ID NO: 32 - protein, 9 aa, Artificial Sequence
  Synthetic Construct
DASNLETGV

SEQ ID NO: 33 - protein, 11 aa, Artificial Sequence
  Synthetic Construct
YCQQSLEDPFT

SEQ ID NO: 34 - protein, 12 aa, Artificial Sequence
  Synthetic Construct
YSISSGHYWAWI

SEQ ID NO: 35 - protein, 20 aa, Artificial Sequence
  Synthetic Construct
IGEIYHSGSTYYSPSLKSRV

SEQ ID NO: 36 - protein, 9 aa, Artificial Sequence
  Synthetic Construct
TRGRYGLDY

SEQ ID NO: 37 - protein, 10 aa, Artificial Sequence
  Synthetic Construct
SASSSVSYVQ

SEQ ID NO: 38 - protein, 9 aa, Artificial Sequence
  Synthetic Construct
DASNRATGI

SEQ ID NO: 39 - protein, 11 aa, Artificial Sequence
  Synthetic Construct
YCQQYSTNPLT

SEQ ID NO: 40 - protein, 13 aa, Artificial Sequence
  Synthetic Construct
FSLSTSGVGVSWI

SEQ ID NO: 41 - protein, 20 aa, Artificial Sequence
  Synthetic Construct
LALIDWAGDKYYSPSLKSRL

SEQ ID NO: 42 - protein, 12 aa, Artificial Sequence
  Synthetic Construct
ARYGYSSYALDY

SEQ ID NO: 43 - protein, 12 aa, Artificial Sequence
  Synthetic Construct
RASQSVRGSYLA

SEQ ID NO: 44 - protein, 9 aa, Artificial Sequence
  Synthetic Construct
AASTLQSGV

SEQ ID NO: 45 - protein, 10 aa, Artificial Sequence
  Synthetic Construct
YCQQYSSLWT

SEQ ID NO: 46 - protein, 12 aa, Artificial Sequence
  Synthetic Construct
YSISSGYHWDWI

SEQ ID NO: 47 - protein, 21 aa, Artificial Sequence
  Synthetic Construct
IGRINPNRGSTKYAQKFQGRV

SEQ ID NO: 48 - protein, 7 aa, Artificial Sequence
  Synthetic Construct
AREFGAY

SEQ ID NO: 49 - protein, 15 aa, Artificial Sequence
  Synthetic Construct
RASQSVDFVGKSFLD

SEQ ID NO: 50 - protein, 9 aa, Artificial Sequence
  Synthetic Construct
DASNLETGV

SEQ ID NO: 51 - protein, 11 aa, Artificial Sequence
  Synthetic Construct
YCQQRASWPLT

SEQ ID NO: 52 - protein, 127 aa, Artificial Sequence
  Synthetic Construct
EVQLVESGGGLVQPGGSLRLSCAASGYSITSGYYWAWIRQAPGKGLEWIGIINPNRGSTKYAQKFQGRVTISRDNSKNTLYLQLNSLRAEDTAVYYCARDGWALSGGYYYYYGLDVWGQGTLVTVSS

SEQ ID NO: 53 - protein, 108 aa, Artificial Sequence
  Synthetic Construct
DIQLTQSPSSLSASVGDRVTITCRASQDISSDLAWYQQKPGKAPKLLIYDASNLETGVPSRFSGSGSGTDFTLTISSLQPEDFATYYCQQYDAWPSTFGQGTKVEIKR

SEQ ID NO: 54 - protein, 118 aa, Artificial Sequence
  Synthetic Construct
EVQLVESGGGLVQPGGSLRLSCAASGYSISSGHYWAWIRQAPGKGLEWVSVISGSGGSTYYADSVKGRFTISRDNSKNTLYLQLNSLRAEDTAVYYCARYRSGAFDYWGQGTLVTVSS

SEQ ID NO: 55 - protein, 107 aa, Artificial Sequence
  Synthetic Construct
DIQLTQSPSSLSASVGDRVTITCRASQSISRWLAWYQQKPGKAPKLLIYAASSLQSGVPSRFSGSGSGTDFTLTISSLQPEDFATYYCQQAYSIWTFGQGTKVEIKR

SEQ ID NO: 56 - protein, 116 aa, Artificial Sequence
  Synthetic Construct
EVQLVESGGGLVQPGGSLRLSCAASGYSITSGHYWGWIRQAPGKGLEWIGEIYHSGSTYYSPSLKSRVTISRDNSKNTLYLQLNSLRAEDTAVYYCTRGRYGLDYWGQGTLVTVSS

SEQ ID NO: 57 - protein, 107 aa, Artificial Sequence
  Synthetic Construct
DIQLTQSPSSLSASVGDRVTITCSASSSVGYVQWYQQKPGKAPKLLIYDASNRATGIPSRFSGSGSGTDFTLTISSLQPEDFATYYCQQYGSAPITFGQGTKVEIKR

SEQ ID NO: 58 - protein, 121 aa, Artificial Sequence
  Synthetic Construct
EVQLVESGGGLVQPGGSLRLSCAASGFTFTGYGIHWVRQAPGKGLEWVSAISGSGGSTYYADSVKGRFTISRDNSKNTLYLQLNSLRAEDTAVYYCARGAYYGGSYYFDYWGQGTLVTVSS

SEQ ID NO: 59 - protein, 109 aa, Artificial Sequence
  Synthetic Construct
DIQLTQSPSSLSASVGDRVTITCRASESITSSYLAWYQQKPGKAPKLLIYDASSLESGVPSRFSGSGSGTDFTLTISSLQPEDFATYYCFQGSHYPPTFGQGTKVEIKR

SEQ ID NO: 60 - protein, 123 aa, Artificial Sequence
  Synthetic Construct
EVQLVESGGGLVQPGGSLRLSCAASGYSISSGHYWSWIRQAPGKGLEWIGEIYHSGSTYYSPSLKSRVTISRDNSKNTLYLQLNSLRAEDTAVYYCAREGSDSYGGPDPFDIWGQGTLVTVSS

SEQ ID NO: 61 - protein, 109 aa, Artificial Sequence
  Synthetic Construct
DIQLTQSPSSLSASVGDRVTITCRARSTVSFPYLHWYQQKPGKAPKLLIYDASNLETGVPSRFSGSGSGTDFTLTISSLQPEDFATYYCQQSLEDPFTFGQGTKVEIKR

SEQ ID NO: 62 - protein, 116 aa, Artificial Sequence
  Synthetic Construct
EVQLVESGGGLVQPGGSLRLSCAASGYSISSGHYWAWIRQAPGKGLEWIGEIYHSGSTYYSPSLKSRVTISRDNSKNTLYLQLNSLRAEDTAVYYCTRGRYGLDYWGQGTLVTVSS

SEQ ID NO: 63 - protein, 107 aa, Artificial Sequence
  Synthetic Construct
DIQLTQSPSSLSASVGDRVTITCSASSSVSYVQWYQQKPGKAPKLLIYDASNRATGIPSRFSGSGSGTDFTLTISSLQPEDFATYYCQQYSTNPLTFGQGTKVEIKR

SEQ ID NO: 64 - protein, 120 aa, Artificial Sequence
  Synthetic Construct
EVQLVESGGGLVQPGGSLRLSCAASGFSLSTSGVGVSWIRQAPGKGLEWLALIDWAGDKYYSPSLKSRLTISRDNSKNTLYLQLNSLRAEDTAVYYCARYGYSSYALDYWGQGTLVTVSS

SEQ ID NO: 65 - protein, 108 aa, Artificial Sequence
  Synthetic Construct
DIQLTQSPSSLSASVGDRVTITCRASQSVRGSYLAWYQQKPGKAPKLLIYAASTLQSGVPSRFSGSGSGTDFTLTISSLQPEDFATYYCQQYSSLWTFGQGTKVEIKR

SEQ ID NO: 66 - protein, 115 aa, Artificial Sequence
  Synthetic Construct
EVQLVESGGGLVQPGGSLRLSCAASGYSISSGYHWDWIRQAPGKGLEWIGRINPNRGSTKYAQKFQGRVTISRDNSKNTLYLQLNSLRAEDTAVYYCAREFGAYWGQGTLVTVSS

SEQ ID NO: 67 - protein, 112 aa, Artificial Sequence
  Synthetic Construct
DIQLTQSPSSLSASVGDRVTITCRASQSVDFVGKSFLDWYQQKPGKAPKLLIYDASNLETGVPSRFSGSGSGTDFTLTISSLQPEDFATYYCQQRASWPLTFGQGTKVEIKR

SEQ ID NO: 68 - protein, 13 aa, Artificial Sequence
  Synthetic Construct
FSLNTAGVGVTWI

SEQ ID NO: 69 - protein, 13 aa, Artificial Sequence
  Synthetic Construct
FSLSTSGVGVAWI

SEQ ID NO: 70 - protein, 13 aa, Artificial Sequence
  Synthetic Construct
FSLSTAGVGVSWI

SEQ ID NO: 71 - protein, 13 aa, Artificial Sequence
  Synthetic Construct
FSLSTSGVGVAWI

SEQ ID NO: 72 - protein, 13 aa, Artificial Sequence
  Synthetic Construct
FSLSTSGVGVGWI

SEQ ID NO: 73 - protein, 13 aa, Artificial Sequence
  Synthetic Construct
FSLSTGGVGVAWI

SEQ ID NO: 74 - protein, 13 aa, Artificial Sequence
  Synthetic Construct
FSLSTGGVGVAWI

SEQ ID NO: 75 - protein, 13 aa, Artificial Sequence
  Synthetic Construct
FSLSTAGVGVTWI

SEQ ID NO: 76 - protein, 13 aa, Artificial Sequence
  Synthetic Construct
FSLSTAGVGVAWI

SEQ ID NO: 77 - protein, 13 aa, Artificial Sequence
  Synthetic Construct
FSLTTTGVGVGWI

SEQ ID NO: 78 - protein, 13 aa, Artificial Sequence
  Synthetic Construct
FSLTTSGVGVSWI

SEQ ID NO: 79 - protein, 13 aa, Artificial Sequence
  Synthetic Construct
FSLSTGGVGVGWI

SEQ ID NO: 80 - protein, 13 aa, Artificial Sequence
  Synthetic Construct
FSLSTGGVGVSWI

SEQ ID NO: 81 - protein, 13 aa, Artificial Sequence
  Synthetic Construct
FSLSTGGVGVTWI

SEQ ID NO: 82 - protein, 13 aa, Artificial Sequence
  Synthetic Construct
FSLTTGGVGVTWI

SEQ ID NO: 83 - protein, 13 aa, Artificial Sequence
  Synthetic Construct
FSLTTAGVGVAWI

SEQ ID NO: 84 - protein, 13 aa, Artificial Sequence
  Synthetic Construct
FSLSTAGVGVTWI

SEQ ID NO: 85 - protein, 13 aa, Artificial Sequence
  Synthetic Construct
FSLNTTGVGVSWI

SEQ ID NO: 86 - protein, 13 aa, Artificial Sequence
  Synthetic Construct
FSLSTAGVGVAWI

SEQ ID NO: 87 - protein, 13 aa, Artificial Sequence
  Synthetic Construct
FSLTTGGVGVAWI

SEQ ID NO: 88 - protein, 13 aa, Artificial Sequence
  Synthetic Construct
FSLSTGGVGVAWI

SEQ ID NO: 89 - protein, 13 aa, Artificial Sequence
  Synthetic Construct
FSLSTGGVGVGWI

SEQ ID NO: 90 - protein, 13 aa, Artificial Sequence
  Synthetic Construct
FSLNTGGVGVTWI

SEQ ID NO: 91 - protein, 13 aa, Artificial Sequence
  Synthetic Construct
FSLSTAGVGVAWI

SEQ ID NO: 92 - protein, 13 aa, Artificial Sequence
  Synthetic Construct
FSLSTAGVGVAWI

SEQ ID NO: 93 - protein, 13 aa, Artificial Sequence
  Synthetic Construct
FSLSTAGVGVAWI

SEQ ID NO: 94 - protein, 13 aa, Artificial Sequence
  Synthetic Construct
FSLSTGGVGVGWI

SEQ ID NO: 95 - protein, 13 aa, Artificial Sequence
  Synthetic Construct
FSLSTAGVGVGWI

SEQ ID NO: 96 - protein, 13 aa, Artificial Sequence
  Synthetic Construct
FSLSTSGVGVAWI

SEQ ID NO: 97 - protein, 13 aa, Artificial Sequence
  Synthetic Construct
FSLSTGGVGVSWI

SEQ ID NO: 98 - protein, 13 aa, Artificial Sequence
  Synthetic Construct
FSLSTSGVGVTWI

SEQ ID NO: 99 - protein, 13 aa, Artificial Sequence
  Synthetic Construct
FSLSTGGVGVAWI

SEQ ID NO: 100 - protein, 13 aa, Artificial Sequence
  Synthetic Construct
FSLSTSGVGVAWI

SEQ ID NO: 101 - protein, 13 aa, Artificial Sequence
  Synthetic Construct
FSLSTSGVGVAWI

SEQ ID NO: 102 - protein, 13 aa, Artificial Sequence
  Synthetic Construct
FSLSTGGVGVAWI

SEQ ID NO: 103 - protein, 13 aa, Artificial Sequence
  Synthetic Construct
FSLSTAGVGVSWI

SEQ ID NO: 104 - protein, 13 aa, Artificial Sequence
  Synthetic Construct
FSLSTGGVGVAWI

SEQ ID NO: 105 - protein, 13 aa, Artificial Sequence
  Synthetic Construct
FSLSTGGVGVAWI

SEQ ID NO: 106 - protein, 13 aa, Artificial Sequence
  Synthetic Construct
FSLSTTGVGVAWI

SEQ ID NO: 107 - protein, 13 aa, Artificial Sequence
  Synthetic Construct
FSLSTAGVGVGWI

SEQ ID NO: 108 - protein, 13 aa, Artificial Sequence
  Synthetic Construct
FSLSTAGVGVAWI

SEQ ID NO: 109 - protein, 13 aa, Artificial Sequence
  Synthetic Construct
FSLSTGGVGVSWI

SEQ ID NO: 110 - protein, 13 aa, Artificial Sequence
  Synthetic Construct
FSLSTAGVGVSWI

SEQ ID NO: 111 - protein, 13 aa, Artificial Sequence
  Synthetic Construct
FSLSTGGVGVAWI

SEQ ID NO: 112 - protein, 13 aa, Artificial Sequence
  Synthetic Construct
FSLNTAGVGVSWI

SEQ ID NO: 113 - protein, 13 aa, Artificial Sequence
  Synthetic Construct
FSLTTSGVGVAWI

SEQ ID NO: 114 - protein, 13 aa, Artificial Sequence
  Synthetic Construct
FSLTTSGVGVGWI

SEQ ID NO: 115 - protein, 13 aa, Artificial Sequence
  Synthetic Construct
FSLSTAGVGVAWI

SEQ ID NO: 116 - protein, 20 aa, Artificial Sequence
  Synthetic Construct
IGLIDWAGYTYYSPSLKSRL

SEQ ID NO: 117 - protein, 20 aa, Artificial Sequence
  Synthetic Construct
LAVIDWAGYKYYSPSLKSRL

SEQ ID NO: 118 - protein, 20 aa, Artificial Sequence
  Synthetic Construct
IAVIDWAGAKYYNPSLKSRL

SEQ ID NO: 119 - protein, 20 aa, Artificial Sequence
  Synthetic Construct
LAVIDWAGYKYYSPSLKSRL

SEQ ID NO: 120 - protein, 20 aa, Artificial Sequence
  Synthetic Construct
IALIDYAGSKYYSPSLKSRL

SEQ ID NO: 121 - protein, 20 aa, Artificial Sequence
  Synthetic Construct
LGLIDWAGYTYYSPSLKSRL

SEQ ID NO: 122 - protein, 20 aa, Artificial Sequence
  Synthetic Construct
IGLIDWAGYTYYSPSLKSRL

SEQ ID NO: 123 - protein, 20 aa, Artificial Sequence
  Synthetic Construct
LALIDWAGDKYYSPSLKSRL

SEQ ID NO: 124 - protein, 20 aa, Artificial Sequence
  Synthetic Construct
LALIDWAGDKYYSPSLKSRL

SEQ ID NO: 125 - protein, 20 aa, Artificial Sequence
  Synthetic Construct
IGLIDYAGYTYYSPSLKSRL

SEQ ID NO: 126 - protein, 20 aa, Artificial Sequence
  Synthetic Construct
LGLIDWSGYTYYNPSLKSRL

SEQ ID NO: 127 - protein, 20 aa, Artificial Sequence
  Synthetic Construct
LALIDWAGDKYYSPSLKSRL

SEQ ID NO: 128 - protein, 20 aa, Artificial Sequence
  Synthetic Construct
IAVIDYAGYTYYSPSLKSRL

SEQ ID NO: 129 - protein, 20 aa, Artificial Sequence
  Synthetic Construct
LALIDWAGDTYYSPSLKSRL

SEQ ID NO: 130 - protein, 20 aa, Artificial Sequence
  Synthetic Construct
LGLIDWSGDKYYNPSLKSRL

SEQ ID NO: 131 - protein, 20 aa, Artificial Sequence
  Synthetic Construct
IGLIDWAGYKYYSPSLKSRL

SEQ ID NO: 132 - protein, 20 aa, Artificial Sequence
  Synthetic Construct
LALIDWAGDKYYSPSLKSRL

SEQ ID NO: 133 - protein, 20 aa, Artificial Sequence
  Synthetic Construct
IGLIDWAGYTYYNPSLKSRL

SEQ ID NO: 134 - protein, 20 aa, Artificial Sequence
  Synthetic Construct
IGLIDWAGSKYYNPSLKSRL

SEQ ID NO: 135 - protein, 20 aa, Artificial Sequence
  Synthetic Construct
LAVIDWAGYKYYSPSLKSRL

SEQ ID NO: 136 - protein, 20 aa, Artificial Sequence
  Synthetic Construct
LALIDWAGDKYYSPSLKSRL

SEQ ID NO: 137 - protein, 20 aa, Artificial Sequence
  Synthetic Construct
LALIDWAGDKYYSPSLKSRL

SEQ ID NO: 138 - protein, 20 aa, Artificial Sequence
  Synthetic Construct
IGLIDWAGYKYYSPSLKSRL

SEQ ID NO: 139 - protein, 20 aa, Artificial Sequence
  Synthetic Construct
LALIDWADDKYYSPSLKSRL

SEQ ID NO: 140 - protein, 20 aa, Artificial Sequence
  Synthetic Construct
LALIDWAGDTYYSPSLKSRL

SEQ ID NO: 141 - protein, 20 aa, Artificial Sequence
  Synthetic Construct
LALIDYAGDKYYNPSPKSHL

SEQ ID NO: 142 - protein, 20 aa, Artificial Sequence
  Synthetic Construct
LAVIDYAGYTYYSPSLKSRL

SEQ ID NO: 143 - protein, 20 aa, Artificial Sequence
  Synthetic Construct
LALIDYAGYTYYSPSLKSRL

SEQ ID NO: 144 - protein, 20 aa, Artificial Sequence
  Synthetic Construct
LALIDYAGYTYYSPSLKSRL

SEQ ID NO: 145 - protein, 20 aa, Artificial Sequence
  Synthetic Construct
LALIDYAGYTYYSPSLKSRL

SEQ ID NO: 146 - protein, 20 aa, Artificial Sequence
  Synthetic Construct
LALIDYAGYKYYSPSLKSRL

SEQ ID NO: 147 - protein, 20 aa, Artificial Sequence
  Synthetic Construct
LALIDWSGDKYYSPSLKSRL

SEQ ID NO: 148 - protein, 20 aa, Artificial Sequence
  Synthetic Construct
LALIDYSGATYYSPSLKSRL

SEQ ID NO: 149 - protein, 20 aa, Artificial Sequence
  Synthetic Construct
LALIDYAGYTYYSPSLKSRL

SEQ ID NO: 150 - protein, 20 aa, Artificial Sequence
  Synthetic Construct
LALIDYSGDKYYSPSPKSRL

SEQ ID NO: 151 - protein, 20 aa, Artificial Sequence
  Synthetic Construct
LALIDYAGSKYYSPSLKSRL

SEQ ID NO: 152 - protein, 20 aa, Artificial Sequence
  Synthetic Construct
LALIDWAGDKYYNPSLKSRL

SEQ ID NO: 153 - protein, 20 aa, Artificial Sequence
  Synthetic Construct
LALIDYAGYKYYSPSLKSRL

SEQ ID NO: 154 - protein, 20 aa, Artificial Sequence
  Synthetic Construct
LAIIDWSGSKYYSPSLKSRL

SEQ ID NO: 155 - protein, 20 aa, Artificial Sequence
  Synthetic Construct
LALIDWSGATYYSPSLKSRL

SEQ ID NO: 156 - protein, 20 aa, Artificial Sequence
  Synthetic Construct
LALIDYAGDKYYNPSPKSHL

SEQ ID NO: 157 - protein, 20 aa, Artificial Sequence
  Synthetic Construct
LALIDWAGDKYYSPSLKSRL

SEQ ID NO: 158 - protein, 20 aa, Artificial Sequence
  Synthetic Construct
LALIDWAGDTYYSPSLKSRL

SEQ ID NO: 159 - protein, 20 aa, Artificial Sequence
  Synthetic Construct
LALIDWAGDKYYSPSLKSRL

SEQ ID NO: 160 - protein, 20 aa, Artificial Sequence
  Synthetic Construct
LALIDWAGYKYYSPSLKSRL

SEQ ID NO: 161 - protein, 20 aa, Artificial Sequence
  Synthetic Construct
IGLIDHAGYTYYSPSLKSRL

SEQ ID NO: 162 - protein, 20 aa, Artificial Sequence
  Synthetic Construct
IGLIDYAGYKYYSPSLKSRL

SEQ ID NO: 163 - protein, 20 aa, Artificial Sequence
  Synthetic Construct
LALIDWAGDTYYSPSLKSRL

SEQ ID NO: 164 - protein, 12 aa, Artificial Sequence
  Synthetic Construct
ARYGYGGYAIDY

SEQ ID NO: 165 - protein, 12 aa, Artificial Sequence
  Synthetic Construct
ARYGYRNYALDY

SEQ ID NO: 166 - protein, 12 aa, Artificial Sequence
  Synthetic Construct
ARYGYRNYALDY

SEQ ID NO: 167 - protein, 12 aa, Artificial Sequence
  Synthetic Construct
ARYGYRNYALDY

SEQ ID NO: 168 - protein, 12 aa, Artificial Sequence
  Synthetic Construct
ARYGYRSYAMDY

SEQ ID NO: 169 - protein, 12 aa, Artificial Sequence
  Synthetic Construct
ARYGYGSYALDY

SEQ ID NO: 170 - protein, 12 aa, Artificial Sequence
  Synthetic Construct
ARYGYRSYAMDY

SEQ ID NO: 171 - protein, 12 aa, Artificial Sequence
  Synthetic Construct
ARYGVGSYALDY

SEQ ID NO: 172 - protein, 12 aa, Artificial Sequence
  Synthetic Construct
ARYGYSSYALDY

SEQ ID NO: 173 - protein, 12 aa, Artificial Sequence
  Synthetic Construct
ARYGYDSYAMDY

SEQ ID NO: 174 - protein, 12 aa, Artificial Sequence
  Synthetic Construct
ARYGYRNYALDY

SEQ ID NO: 175 - protein, 12 aa, Artificial Sequence
  Synthetic Construct
ARYGWSSYALDY

SEQ ID NO: 176 - protein, 12 aa, Artificial Sequence
  Synthetic Construct
ARYGYNSYALDY

SEQ ID NO: 177 - protein, 12 aa, Artificial Sequence
  Synthetic Construct
ARYGVSSYALDY

SEQ ID NO: 178 - protein, 12 aa, Artificial Sequence
  Synthetic Construct
ARYGYDSYAMDY

SEQ ID NO: 179 - protein, 12 aa, Artificial Sequence
  Synthetic Construct
ARYGYDGYAMDY

SEQ ID NO: 180 - protein, 12 aa, Artificial Sequence
  Synthetic Construct
ARYGYSSYALDY

SEQ ID NO: 181 - protein, 12 aa, Artificial Sequence
  Synthetic Construct
ARYGYSGYALDY

SEQ ID NO: 182 - protein, 12 aa, Artificial Sequence
  Synthetic Construct
ARYGYSGYAMDY

SEQ ID NO: 183 - protein, 12 aa, Artificial Sequence
  Synthetic Construct
ARYGYNSYALDY

SEQ ID NO: 184 - protein, 12 aa, Artificial Sequence
  Synthetic Construct
ARYGYGSYALDY

SEQ ID NO: 185 - protein, 12 aa, Artificial Sequence
  Synthetic Construct
ARYGYGSYALDY

SEQ ID NO: 186 - protein, 12 aa, Artificial Sequence
  Synthetic Construct
ARYGYDSYALDY

SEQ ID NO: 187 - protein, 12 aa, Artificial Sequence
  Synthetic Construct
ARYGYSNYALDY

SEQ ID NO: 188 - protein, 12 aa, Artificial Sequence
  Synthetic Construct
ARYGLSSYALDY

SEQ ID NO: 189 - protein, 12 aa, Artificial Sequence
  Synthetic Construct
ARYGYSGYALDY

SEQ ID NO: 190 - protein, 12 aa, Artificial Sequence
  Synthetic Construct
ARYGYGSYALDY

SEQ ID NO: 191 - protein, 12 aa, Artificial Sequence
  Synthetic Construct
ARYGYSSYALDY

SEQ ID NO: 192 - protein, 12 aa, Artificial Sequence
  Synthetic Construct
ARYGYSSYALDY

SEQ ID NO: 193 - protein, 12 aa, Artificial Sequence
  Synthetic Construct
ARYGYSGYALDY

SEQ ID NO: 194 - protein, 12 aa, Artificial Sequence
  Synthetic Construct
ARYGYGSYALDY

SEQ ID NO: 195 - protein, 12 aa, Artificial Sequence
  Synthetic Construct
ARYGYGSYALDY

SEQ ID NO: 196 - protein, 12 aa, Artificial Sequence
  Synthetic Construct
ARYGYGSYALDY

SEQ ID NO: 197 - protein, 12 aa, Artificial Sequence
  Synthetic Construct
ARYGYSGYALDY

SEQ ID NO: 198 - protein, 12 aa, Artificial Sequence
  Synthetic Construct
ARYGYGSYALDY

SEQ ID NO: 199 - protein, 12 aa, Artificial Sequence
  Synthetic Construct
ARYGYGSYALDY

SEQ ID NO: 200 - protein, 12 aa, Artificial Sequence
  Synthetic Construct
ARYGYGSYALDY

SEQ ID NO: 201 - protein, 12 aa, Artificial Sequence
  Synthetic Construct
ARYGYGGYALDY

SEQ ID NO: 202 - protein, 12 aa, Artificial Sequence
  Synthetic Construct
ARYGYSSYALDY

SEQ ID NO: 203 - protein, 12 aa, Artificial Sequence
  Synthetic Construct
ARYGYSGYALDY

SEQ ID NO: 204 - protein, 12 aa, Artificial Sequence
  Synthetic Construct
ARYGYSGYALDY

SEQ ID NO: 205 - protein, 12 aa, Artificial Sequence
  Synthetic Construct
ARYGYGSYALDY

SEQ ID NO: 206 - protein, 12 aa, Artificial Sequence
  Synthetic Construct
ARYGVSSYALDY

SEQ ID NO: 207 - protein, 12 aa, Artificial Sequence
  Synthetic Construct
ARYGGSSYALDY

SEQ ID NO: 208 - protein, 12 aa, Artificial Sequence
  Synthetic Construct
ARYGYRNYAMDY

SEQ ID NO: 209 - protein, 12 aa, Artificial Sequence
  Synthetic Construct
ARYGYDSYAMDY

SEQ ID NO: 210 - protein, 12 aa, Artificial Sequence
  Synthetic Construct
ARYGYRGYALDY

SEQ ID NO: 211 - protein, 12 aa, Artificial Sequence
  Synthetic Construct
ARYGVGSYALDY

SEQ ID NO: 212 - protein, 12 aa, Artificial Sequence
  Synthetic Construct
RASQSVRGSYLA

SEQ ID NO: 213 - protein, 12 aa, Artificial Sequence
  Synthetic Construct
RASQSVRGNYLA

SEQ ID NO: 214 - protein, 12 aa, Artificial Sequence
  Synthetic Construct
RASQSVSGSYLA

SEQ ID NO: 215 - protein, 12 aa, Artificial Sequence
  Synthetic Construct
RASQTIRGSYLA

SEQ ID NO: 216 - protein, 12 aa, Artificial Sequence
  Synthetic Construct
RASQSVRNSYLA

SEQ ID NO: 217 - protein, 12 aa, Artificial Sequence
  Synthetic Construct
RASQTVRGTFLA

SEQ ID NO: 218 - protein, 12 aa, Artificial Sequence
  Synthetic Construct
RASQSVRGTFLA

SEQ ID NO: 219 - protein, 12 aa, Artificial Sequence
  Synthetic Construct
RASQTVRGSYLA

SEQ ID NO: 220 - protein, 12 aa, Artificial Sequence
  Synthetic Construct
RASQTVRGNYLA

SEQ ID NO: 221 - protein, 12 aa, Artificial Sequence
  Synthetic Construct
RASQSIRGNYLA

SEQ ID NO: 222 - protein, 12 aa, Artificial Sequence
  Synthetic Construct
RTSQSVRGSYLA

SEQ ID NO: 223 - protein, 12 aa, Artificial Sequence
  Synthetic Construct
RASQSIRGSYLA

SEQ ID NO: 224 - protein, 12 aa, Artificial Sequence
  Synthetic Construct
RASQSIHGSYLA

SEQ ID NO: 225 - protein, 12 aa, Artificial Sequence
  Synthetic Construct
RASQSVHGSYLA

SEQ ID NO: 226 - protein, 11 aa, Artificial Sequence
  Synthetic Construct
RASQGVSSYLA

SEQ ID NO: 227 - protein, 12 aa, Artificial Sequence
  Synthetic Construct
RASQTVRGTYLA

SEQ ID NO: 228 - protein, 9 aa, Artificial Sequence
  Synthetic Construct
AASTLQSGV

SEQ ID NO: 229 - protein, 9 aa, Artificial Sequence
  Synthetic Construct
DASSLESGV

SEQ ID NO: 230 - protein, 9 aa, Artificial Sequence
  Synthetic Construct
AAFTLQSGV

SEQ ID NO: 231 - protein, 9 aa, Artificial Sequence
  Synthetic Construct
AASSLQSGV

SEQ ID NO: 232 - protein, 10 aa, Artificial Sequence
  Synthetic Construct
YCQQYSSLWT

SEQ ID NO: 233 - protein, 10 aa, Artificial Sequence
  Synthetic Construct
YCQQYGSLWT

SEQ ID NO: 234 - protein, 10 aa, Artificial Sequence
  Synthetic Construct
YCQQYGSYWT

SEQ ID NO: 235 - protein, 10 aa, Artificial Sequence [sequence omitted]

SEQ ID NO: 236 - protein, 10 aa, Artificial Sequence
  Synthetic Construct
YCQQYNSSWT

SEQ ID NO: 237 - protein, 10 aa, Artificial Sequence
  Synthetic Construct
YCQQYSSSWT

SEQ ID NO: 238 - protein, 10 aa, Artificial Sequence
  Synthetic Construct
YCQQYNSLWT

SEQ ID NO: 239 - protein, 10 aa, Artificial Sequence
  Synthetic Construct
YCQQYSSWWT

SEQ ID NO: 240 - protein, 10 aa, Artificial Sequence
  Synthetic Construct
YCQQYSSFWT

SEQ ID NO: 241 - protein, 120 aa, Artificial Sequence
  Synthetic Construct
EVQLVESGGGLVQPGGSLRLSCAASGFSLNTAGVGVTWIRQAPGKGLEWIGLIDWAGYTYYSPSLKSRLTISRDNSKNTLYLQLNSLRAEDTAVYYCARYGYGGYAIDYWGQGTLVTVSS

SEQ ID NO: 242 - protein, 108 aa, Artificial Sequence; identical to SEQ ID NO: 65
  Synthetic Construct

SEQ ID NO: 243 - protein, 120 aa, Artificial Sequence
  Synthetic Construct
EVQLVESGGGLVQPGGSLRLSCAASGFSLSTSGVGVAWIRQAPGKGLEWLAVIDWAGYKYYSPSLKSRLTISRDNSKNTLYLQLNSLRAEDTAVYYCARYGYRNYALDYWGQGTLVTVSS

SEQ ID NO: 244 - protein, 108 aa, Artificial Sequence
  Synthetic Construct
DIQLTQSPSSLSASVGDRVTITCRASQSVRGSYLAWYQQKPGKAPKLLIYAASTLQSGVPSRFSGSGSGTDFTLTISSLQPEDFATYYCQQYGSLWTFGQGTKVEIKR

SEQ ID NO: 245 - protein, 120 aa, Artificial Sequence
  Synthetic Construct
EVQLVESGGGLVQPGGSLRLSCAASGFSLSTAGVGVSWIRQAPGKGLEWIAVIDWAGAKYYNPSLKSRLTISRDNSKNTLYLQLNSLRAEDTAVYYCARYGYRNYALDYWGQGTLVTVSS

SEQ ID NO: 246 - protein, 108 aa, Artificial Sequence
  Synthetic Construct
DIQLTQSPSSLSASVGDRVTITCRASQSVRGNYLAWYQQKPGKAPKLLIYAASTLQSGVPSRFSGSGSGTDFTLTISSLQPEDFATYYCQQYGSLWTFGQGTKVEIKR

SEQ ID NO: 247 - protein, 120 aa, Artificial Sequence; identical to SEQ ID NO: 243
  Synthetic Construct

SEQ ID NO: 248 - protein, 108 aa, Artificial Sequence
  Synthetic Construct
DIQLTQSPSSLSASVGDRVTITCRASQSVRGSYLAWYQQKPGKAPKLLIYAASTLQSGVPSRFSGSGSGTDFTLTISSLQPEDFATYYCQQYGSYWTFGQGTKVEIKR

SEQ ID NO: 249 - protein, 120 aa, Artificial Sequence
  Synthetic Construct
EVQLVESGGGLVQPGGSLRLSCAASGFSLSTSGVGVGWIRQAPGKGLEWIALIDYAGSKYYSPSLKSRLTISRDNSKNTLYLQLNSLRAEDTAVYYCARYGYRSYAMDYWGQGTLVTVSS

SEQ ID NO: 250 - protein, 108 aa, Artificial Sequence
  Synthetic Construct
DIQLTQSPSSLSASVGDRVTITCRASQSVRGNYLAWYQQKPGKAPKLLIYDASSLESGVPSRFSGSGSGTDFTLTISSLQPEDFATYYCQQYSSYWTFGQGTKVEIKR

SEQ ID NO: 251 - protein, 120 aa, Artificial Sequence
  Synthetic Construct
EVQLVESGGGLVQPGGSLRLSCAASGFSLSTGGVGVAWIRQAPGKGLEWLGLIDWAGYTYYSPSLKSRLTISRDNSKNTLYLQLNSLRAEDTAVYYCARYGYGSYALDYWGQGTLVTVSS

SEQ ID NO: 252 - protein, 108 aa, Artificial Sequence
  Synthetic Construct
DIQLTQSPSSLSASVGDRVTITCRASQSVRGSYLAWYQQKPGKAPKLLIYDASSLESGVPSRFSGSGSGTDFTLTISSLQPEDFATYYCQQYSSLWTFGQGTKVEIKR

SEQ ID NO: 253 - protein, 120 aa, Artificial Sequence
  Synthetic Construct
EVQLVESGGGLVQPGGSLRLSCAASGFSLSTGGVGVAWIRQAPGKGLEWIGLIDWAGYTYYSPSLKSRLTISRDNSKNTLYLQLNSLRAEDTAVYYCARYGYRSYAMDYWGQGTLVTVSS

SEQ ID NO: 254 - protein, 108 aa, Artificial Sequence
  Synthetic Construct
DIQLTQSPSSLSASVGDRVTITCRASQSVSGSYLAWYQQKPGKAPKLLIYDASSLESGVPSRFSGSGSGTDFTLTISSLQPEDFATYYCQQYNSSWTFGQGTKVEIKR

SEQ ID NO: 255 - protein, 120 aa, Artificial Sequence
  Synthetic Construct
EVQLVESGGGLVQPGGSLRLSCAASGFSLSTAGVGVTWIRQAPGKGLEWLALIDWAGDKYYSPSLKSRLTISRDNSKNTLYLQLNSLRAEDTAVYYCARYGVGSYALDYWGQGTLVTVSS

SEQ ID NO: 256 - protein, 108 aa, Artificial Sequence
  Synthetic Construct
DIQLTQSPSSLSASVGDRVTITCRASQTIRGSYLAWYQQKPGKAPKLLIYDASSLESGVPSRFSGSGSGTDFTLTISSLQPEDFATYYCQQYSSLWTFGQGTKVEIKR

SEQ ID NO: 257 - protein, 120 aa, Artificial Sequence
  Synthetic Construct
EVQLVESGGGLVQPGGSLRLSCAASGFSLSTAGVGVAWIRQAPGKGLEWLALIDWAGDKYYSPSLKSRLTISRDNSKNTLYLQLNSLRAEDTAVYYCARYGYSSYALDYWGQGTLVTVSS

SEQ ID NO: 258 - protein, 108 aa, Artificial Sequence
  Synthetic Construct
DIQLTQSPSSLSASVGDRVTITCRASQSVRGSYLAWYQQKPGKAPKLLIYDASSLESGVPSRFSGSGSGTDFTLTISSLQPEDFATYYCQQYSSYWTFGQGTKVEIKR

SEQ ID NO: 259 - protein, 120 aa, Artificial Sequence
  Synthetic Construct
EVQLVESGGGLVQPGGSLRLSCAASGFSLTTTGVGVGWIRQAPGKGLEWIGLIDYAGYTYYSPSLKSRLTISRDNSKNTLYLQLNSLRAEDTAVYYCARYGYDSYAMDYWGQGTLVTVSS

SEQ ID NO: 260 - protein, 108 aa, Artificial Sequence; identical to SEQ ID NO: 250
  Synthetic Construct

SEQ ID NO: 261 - protein, 120 aa, Artificial Sequence
  Synthetic Construct
EVQLVESGGGLVQPGGSLRLSCAASGFSLTTSGVGVSWIRQAPGKGLEWLGLIDWSGYTYYNPSLKSRLTISRDNSKNTLYLQLNSLRAEDTAVYYCARYGYRNYALDYWGQGTLVTVSS

SEQ ID NO: 262 - protein, 108 aa, Artificial Sequence
  Synthetic Construct
DIQLTQSPSSLSASVGDRVTITCRASQSVRNSYLAWYQQKPGKAPKLLIYDASSLESGVPSRFSGSGSGTDFTLTISSLQPEDFATYYCQQYSSSWTFGQGTKVEIKR

SEQ ID NO: 263 - protein, 120 aa, Artificial Sequence
  Synthetic Construct
EVQLVESGGGLVQPGGSLRLSCAASGFSLSTGGVGVGWIRQAPGKGLEWLALIDWAGDKYYSPSLKSRLTISRDNSKNTLYLQLNSLRAEDTAVYYCARYGWSSYALDYWGQGTLVTVSS

SEQ ID NO: 264 - protein, 108 aa, Artificial Sequence
  Synthetic Construct
DIQLTQSPSSLSASVGDRVTITCRASQTVRGTFLAWYQQKPGKAPKLLIYDASSLESGVPYRFSGSGSGTDFTLTISSLQPEDFATYYCQQYSSLWTFGQGTKVEIKR

SEQ ID NO: 265 - protein, 120 aa, Artificial Sequence
  Synthetic Construct
EVQLVESGGGLVQPGGSLRLSCAASGFSLSTGGVGVSWIRQAPGKGLEWIAVIDYAGYTYYSPSLKSRLTISRDNSKNTLYLQLNSLRAEDTAVYYCARYGYNSYALDYWGQGTLVTVSS

SEQ ID NO: 266 - protein, 108 aa, Artificial Sequence
  Synthetic Construct
DIQLTQSPSSLSASVGDRVTITCRASQSVRGSYLAWYQQKPGKAPKLLIYDASSLESGVPSRFSGSGSGTDFTLTISSLQPEDFATYYCQQYNSLWTFGQGTKVEIKR

SEQ ID NO: 267 - protein, 120 aa, Artificial Sequence
  Synthetic Construct
EVQLVESGGGLVQPGGSLRLSCAASGFSLSTGGVGVTWIRQAPGKGLEWLALIDWAGDTYYSPSLKSRLTISRDNSKNTLYLQLNSLRAEDTAVYYCARYGVSSYALDYWGQGTLVTVSS

SEQ ID NO: 268 - protein, 108 aa, Artificial Sequence; identical to SEQ ID NO: 264
  Synthetic Construct

SEQ ID NO: 269 - protein, 120 aa, Artificial Sequence
  Synthetic Construct
EVQLVESGGGLVQPGGSLRLSCAASGFSLTTGGVGVTWIRQAPGKGLEWLGLIDWSGDKYYNPSLKSRLTISRDNSKNTLYLQLNSLRAEDTAVYYCARYGYDSYAMDYWGQGTLVTVSS

SEQ ID NO: 270 - protein, 108 aa, Artificial Sequence; identical to SEQ ID NO: 252
  Synthetic Construct

SEQ ID NO: 271 - protein, 120 aa, Artificial Sequence
  Synthetic Construct
EVQLVESGGGLVQPGGSLRLSCAASGFSLTTAGVGVAWIRQAPGKGLEWIGLIDWAGYKYYSPSLKSRLTISRDNSKNTLYLQLNSLRAEDTAVYYCARYGYDGYAMDYWGQGTLVTVSS

SEQ ID NO: 272 - protein, 108 aa, Artificial Sequence
  Synthetic Construct
DIQLTQSPSSLSASVGDRVTITCRASQSVRGNYLAWYQQKPGKAPKLLIYDASSLESGVPSRFSGSGSGTDFTLTISSLQPEDFATYYCQQYSSSWTFGQGTKVEIKR

SEQ ID NO: 273 - protein, 120 aa, Artificial Sequence
  Synthetic Construct
EVQLVESGGGLVQPGGSLRLSCAASGFSLSTAGVGVTWIRQAPGKGLEWLALIDWAGDKYYSPSLKSRLTISRDNSKNTLYLQLNSLRAEDTAVYYCARYGYSSYALDYWGQGTLVTVSS

SEQ ID NO: 274 - protein, 108 aa, Artificial Sequence
  Synthetic Construct
DIQLTQSPSSLSASVGDRVTITCRASQSVRGTFLAWYQQKPGKAPKLLIYDASSLESGVPSRFSGSGSGTDFTLTISSLQPEDFATYYCQQYSSWWTFGQGTKVEIKR

SEQ ID NO: 275 - protein, 120 aa, Artificial Sequence
  Synthetic Construct
EVQLVESGGGLVQPGGSLRLSCAASGFSLNTTGVGVSWIRQAPGKGLEWIGLIDWAGYTYYNPSLKSRLTISRDNSKNTLYLQLNSLRAEDTAVYYCARYGYSGYALDYWGQGTLVTVSS

SEQ ID NO: 276 - protein, 108 aa, Artificial Sequence; identical to SEQ ID NO: 252
  Synthetic Construct

SEQ ID NO: 277 - protein, 120 aa, Artificial Sequence
  Synthetic Construct
EVQLVESGGGLVQPGGSLRLSCAASGFSLSTAGVGVAWIRQAPGKGLEWIGLIDWAGSKYYNPSLKSRLTISRDNSKNTLYLQLNSLRAEDTAVYYCARYGYSGYAMDYWGQGTLVTVSS

SEQ ID NO: 278 - protein, 108 aa, Artificial Sequence
  Synthetic Construct
DIQLTQSPSSLSASVGDRVTITCRASQSVRGSYLAWYQQKPGKAPKLLIYDASSLESGVPSRFSGSGSGTDFTLTISSLQPEDFATYYCQQYSSSWTFGQGTKVEIKR

SEQ ID NO: 279 - protein, 120 aa, Artificial Sequence
  Synthetic Construct
EVQLVESGGGLVQPGGSLRLSCAASGFSLTTGGVGVAWIRQAPGKGLEWLAVIDWAGYKYYSPSLKSRLTISRDNSKNTLYLQLNSLRAEDTAVYYCARYGYNSYALDYWGQGTLVTVSS

SEQ ID NO: 280 - protein, 108 aa, Artificial Sequence
  Synthetic Construct
DIQLTQSPSSLSASVGDRVTITCRASQSVRGSYLAWYQQKPGKAPKLLIYDASSLESGVPSRFSGSGSGTDFTLTISSLQPEDFATYYCQQYSSFWTFGQGTKVEIKR

SEQ ID NO: 281 - protein, 120 aa, Artificial Sequence
  Synthetic Construct
EVQLVESGGGLVQPGGSLRLSCAASGFSLSTGGVGVAWIRQAPGKGLEWLALIDWAGDKYYSPSLKSRLTISRDNSKNTLYLQLNSLRAEDTAVYYCARYGYGSYALDYWGQGTLVTVSS

SEQ ID NO: 282 - protein, 108 aa, Artificial Sequence; identical to SEQ ID NO: 264
  Synthetic Construct

SEQ ID NO: 283 - protein, 120 aa, Artificial Sequence
  Synthetic Construct
EVQLVESGGGLVQPGGSLRLSCAASGFSLSTGGVGVGWIRQAPGKGLEWLALIDWAGDKYYSPSLKSRLTISRDNSKNTLYLQLNSLRAEDTAVYYCARYGYGSYALDYWGQGTLVTVSS

SEQ ID NO: 284 - protein, 108 aa, Artificial Sequence
  Synthetic Construct
DIQLTQSPSSLYASVGDRVTITCRASQTVRGSYLAWYQQKPGKAPKLLIYDASSLESGVPSRFSGSGSGTDFTLTISSLQPEDFATYYCQQYSSLWTFGQGTKVEIKR

SEQ ID NO: 285 - protein, 120 aa, Artificial Sequence
  Synthetic Construct
EVQLVESGGGLVQPGGSLRLSCAASGFSLNTGGVGVTWIRQAPGKGLEWIGLIDWAGYKYYSPSLKSRLTISRDNSKNTLYLQLNSLRAEDTAVYYCARYGYDSYALDYWGQGTLVTVSS

SEQ ID NO: 286 - protein, 108 aa, Artificial Sequence; identical to SEQ ID NO: 272
  Synthetic Construct

SEQ ID NO: 287 - protein, 120 aa, Artificial Sequence
  Synthetic Construct
EVQLVESGGGLVQPGGSLRLSCAASGFSLSTAGVGVAWIRQAPGKGLEWLALIDWADDKYYSPSLKSRLTISRDNSKNTLYLQLNSLRAEDTAVYYCARYGYSNYALDYWGQGTLVTVSS

SEQ ID NO: 288 - protein, 108 aa, Artificial Sequence
  Synthetic Construct
DIQLTQSPSSLSASVGDRVTITCRASQTVRGNYLAWYQQKPGKAPKLLIYAASTLQSGVPSRFSGSGSGTDFTLTISSLQPEDFATYYCQQYGSLWTFGQGTKVEIKR

SEQ ID NO: 289 - protein, 120 aa, Artificial Sequence
  Synthetic Construct
EVQLVESGGGLVQPGGSLRLSCAASGFSLSTAGVGVAWIRQAPGKGLEWLALIDWAGDTYYSPSLKSRLTISRDNSKNTLYLQLNSLRAEDTAVYYCARYGLSSYALDYWGQGTLVTVSS

SEQ ID NO: 290 - protein, 108 aa, Artificial Sequence; identical to SEQ ID NO: 264
  Synthetic Construct

SEQ ID NO: 291 - protein, 120 aa, Artificial Sequence
  Synthetic Construct
EVQLVESGGGLVQPGGSLRLSCAASGFSLSTAGVGVAWIRQAPGKGLEWLALIDYAGDKYYNPSPKSHLTISRDNSKNTLYLQLNSLRAEDTAVYYCARYGYSGYALDYWGQGTLVTVSS

SEQ ID NO: 292 - protein, 108 aa, Artificial Sequence; identical to SEQ ID NO: 272
  Synthetic Construct

SEQ ID NO: 293 - protein, 120 aa, Artificial Sequence
  Synthetic Construct
EVQLVESGGGLVQPGGSLRLSCAASGFSLSTGGVGVGWIRQAPGKGLEWLAVIDYAGYTYYSPSLKSRLTISRDNSKNTLYLQLNSLRAEDTAVYYCARYGYGSYALDYWGQGTLVTVSS

SEQ ID NO: 294 - protein, 108 aa, Artificial Sequence; identical to SEQ ID NO: 278
  Synthetic Construct

SEQ ID NO: 295 - protein, 120 aa, Artificial Sequence
  Synthetic Construct
EVQLVESGGGLVQPGGSLRLSCAASGFSLSTAGVGVGWIRQAPGKGLEWLALIDYAGYTYYSPSLKSRLTISRDNSKNTLYLQLNSLRAEDTAVYYCARYGYSSYALDYWGQGTLVTVSS

SEQ ID NO: 296 - protein, 108 aa, Artificial Sequence; identical to SEQ ID NO: 258
  Synthetic Construct

SEQ ID NO: 297 - protein, 120 aa, Artificial Sequence
  Synthetic Construct
EVQLVESGGGLVQPGGSLRLSCAASGFSLSTSGVGVAWIRQAPGKGLEWLALIDYAGYTYYSPSLKSRLTISRDNSKNTLYLQLNSLRAEDTAVYYCARYGYSSYALDYWGQGTLVTVSS

SEQ ID NO: 298 - protein, 108 aa, Artificial Sequence; identical to SEQ ID NO: 278
  Synthetic Construct

SEQ ID NO: 299 - protein, 120 aa, Artificial Sequence
  Synthetic Construct
EVQLVESGGGLVQPGGSLRLSCAASGFSLSTGGVGVSWIRQAPGKGLEWLALIDYAGYTYYSPSLKSRLTISRDNSKNTLYLQLNSLRAEDTAVYYCARYGYSGYALDYWGQGTLVTVSS

SEQ ID NO: 300 - protein, 108 aa, Artificial Sequence
  Synthetic Construct
DIQLTQSPSSLSASVGDRVTITCRASQSIRGNYLAWYQQKPGKAPKLLIYAAFTLQSGVPSRFSGSGSGTDFTLTISSLQPEDFATYYCQQYSSSWTFGQGTKVEIKR

SEQ ID NO: 301 - protein, 120 aa, Artificial Sequence
  Synthetic Construct
EVQLVESGGGLVQPGGSLRLSCAASGFSLSTSGVGVTWIRQAPGKGLEWLALIDYAGYKYYSPSLKSRLTISRDNSKNTLYLQLNSLRAEDTAVYYCARYGYGSYALDYWGQGTLVTVSS

SEQ ID NO: 302 - protein, 108 aa, Artificial Sequence
  Synthetic Construct
DIQLTQSPSSLSASVGDRVTITCRTSQSVRGSYLAWYQQKPGKAPKLLIYDASSLESGVPSRFSGSGSGTDFTLTISSLQPEDFATYYCQQYSSSWTFGQGTKVEIKR

SEQ ID NO: 303 - protein, 120 aa, Artificial Sequence
  Synthetic Construct
EVQLVESGGGLVQPGGSLRLSCAASGFSLSTGGVGVAWIRQAPGKGLEWLALIDWSGDKYYSPSLKSRLTISRDNSKNTLYLQLNSLRAEDTAVYYCARYGYGSYALDYWGQGTLVTVSS

SEQ ID NO: 304 - protein, 108 aa, Artificial Sequence
  Synthetic Construct
DIQLTQSPSSLSASVGDRVTITCRASQSIRGSYLAWYQQKPGKAPKLLIYDASSLESGVPSRFSGSGSGTDFTLTISSLQPEDFATYYCQQYSSSWTFGQGTKVEIKR

SEQ ID NO: 305 - protein, 120 aa, Artificial Sequence
  Synthetic Construct
EVQLVESGGGLVQPGGSLRLSCAASGFSLSTSGVGVAWIRQAPGKGLEWLALIDYSGATYYSPSLKSRLTISRDNSKNTLYLQLNSLRAEDTAVYYCARYGYGSYALDYWGQGTLVTVSS

SEQ ID NO: 306 - protein, 108 aa, Artificial Sequence; identical to SEQ ID NO: 304
  Synthetic Construct

SEQ ID NO: 307 - protein, 120 aa, Artificial Sequence
  Synthetic Construct
EVQLVESGGGLVQPGGSLRLSCAASGFSLSTSGVGVAWIRQAPGKGLEWLALIDYAGYTYYSPSLKSRLTISRDNSKNTLYLQLNSLRAEDTAVYYCARYGYSGYALDYWGQGTLVTVSS

SEQ ID NO: 308 - protein, 108 aa, Artificial Sequence
  Synthetic Construct
DIQLTQSPSSLSASVGDRVTITCRASQSIHGSYLAWYQQKPGKAPKLLIYDASSLESGVPSRFSGSGSGTDFTLTISSLQPEDFATYYCQQYSSSWTFGQGTKVEIKR

SEQ ID NO: 309 - protein, 120 aa, Artificial Sequence
  Synthetic Construct
EVQLVESGGGLVQPGGSLRLSCAASGFSLSTGGVGVAWIRQAPGKGLEWLALIDYSGDKYYSPSPKSRLTISRDNSKNTLYLQLNSLRAEDTAVYYCARYGYGSYALDYWGQGTLVTVSS

SEQ ID NO: 310 - protein, 108 aa, Artificial Sequence; identical to SEQ ID NO: 280
  Synthetic Construct

SEQ ID NO: 311 - protein, 120 aa, Artificial Sequence
  Synthetic Construct
EVQLVESGGGLVQPGGSLRLSCAASGFSLSTAGVGVSWIRQAPGKGLEWLALIDYAGSKYYSPSLKSRLTISRDNSKNTLYLQLNSLRAEDTAVYYCARYGYGSYALDYWGQGTLVTVSS

SEQ ID NO: 312 - protein, 108 aa, Artificial Sequence
  Synthetic Construct
YIQLTQSPSSLSASVGDRVTITCRASQSIRGSYLAWYQQKPGKAPKLLIYDASSLESGVPSRFSGSGSGTDFTLTISSLQPEDFATYYCQQYSSLWTFGQGTKVEIKR

SEQ ID NO: 313 - protein, 120 aa, Artificial Sequence
  Synthetic Construct
EVQLVESGGGLVQPGGSLRLSCAASGFSLSTGGVGVAWIRQAPGKGLEWLALIDWAGDKYYNPSLKSRLTISRDNSKNTLYLQLNSLRAEDTAVYYCARYGYGSYALDYWGQGTLVTVSS

SEQ ID NO: 314 - protein, 108 aa, Artificial Sequence
  Synthetic Construct
DIQLTQSPSSLSASVGDRVTITCRASQSIRGSYLAWYQQKPGKAPKLLIYDASSLESGVPSRFSGSGSGTDFTLTISSLQPEDFATYYCQQYSSFWTFGQGTKVEIKR

SEQ ID NO: 315 - protein, 120 aa, Artificial Sequence
  Synthetic Construct
EVQLVESGGGLVQPGGSLRLSCAASGFSLSTGGVGVAWIRQAPGKGLEWLALIDYAGYKYYSPSLKSRLTISRDNSKNTLYLQLNSLRAEDTAVYYCARYGYGGYALDYWGQGTLVTVSS

SEQ ID NO: 316 - protein, 108 aa, Artificial Sequence
  Synthetic Construct
DIQLTQSPSSLSASVGDRVTITCRASQSVRGNYLAWYQQKPGKAPKLLIYDASSLESGVPSRFSGSGSGTDFTLTISSLQPEDFATYYCQQYSSFWTFGQGTKVEIKR

SEQ ID NO: 317 - protein, 120 aa, Artificial Sequence
  Synthetic Construct
EVQLVESGGGLVQPGGSLRLSCAASGFSLSTTGVGVAWIRQAPGKGLEWLAIIDWSGSKYYSPSLKSRLTISRDNSKNTLYLQLNSLRAEDTAVYYCARYGYSSYALDYWGQGTLVTVSS

SEQ ID NO: 318 - protein, 108 aa, Artificial Sequence
  Synthetic Construct
DIQLTQSPSSLSASVGDRVTITCRASQSVHGSYLAWYQQKPGKAPKLLIYDASSLESGVPSRFSGSGSGTDFTLTISSLQPEDFATYYCQQYNSLWTFGQGTKVEIKR

SEQ ID NO: 319 - protein, 120 aa, Artificial Sequence
  Synthetic Construct
EVQLVESGGGLVQPGGSLRLSCAASGFSLSTAGVGVGWIRQAPGKGLEWLALIDWSGATYYSPSLKSRLTISRDNSKNTLYLQLNSLRAEDTAVYYCARYGYSGYALDYWGQGTLVTVSS

SEQ ID NO: 320 - protein, 107 aa, Artificial Sequence
  Synthetic Construct
DIQLTQSPSSLSASVGDRVTITCRASQGVSSYLAWYQQKPGKAPKLLIYDASSLESGVPSRFSGSGSGTDFTLTISSLQPEDFATYYCQQYGSYWTFGQGTKVEIKR

SEQ ID NO: 321 - protein, 120 aa, Artificial Sequence; identical to SEQ ID NO: 291
  Synthetic Construct

SEQ ID NO: 322 - protein, 108 aa, Artificial Sequence
  Synthetic Construct
DIQLTQSPSSLSASVGDRVTITCRASQSIRGNYLAWYQQKPGKAPKLLIYDASSLESGVPSRFSGSGSGTDFTLTISSLQPEDFATYYCQQYSSSWTFGQGTKVEIKR

SEQ ID NO: 323 - protein, 120 aa, Artificial Sequence
  Synthetic Construct
EVQLVESGGGLVQPGGSLRLSCAASGFSLSTGGVGVSWIRQAPGKGLEWLALIDWAGDKYYSPSLKSRLTISRDNSKNTLYLQLNSLRAEDTAVYYCARYGYGSYALDYWGQGTLVTVSS

SEQ ID NO: 324 - protein, 108 aa, Artificial Sequence
  Synthetic Construct
DIQLTQSPSSLSASVGDRVTITCRASQTVRGSYLAWYQQKPGKAPKLLIYDASSLESGVPSRFSGSGSGTDFTLTISSLQPEDFATYYCQQYSSLWTFGQGTKVEIKR

SEQ ID NO: 325 - protein, 120 aa, Artificial Sequence
  Synthetic Construct
EVQLVESGGGLVQPGGSLRLSCAASGFSLSTAGVGVSWIRQAPGKGLEWLALIDWAGDTYYSPSLKSRLTISRDNSKNTLYLQLNSLRAEDTAVYYCARYGVSSYALDYWGQGTLVTVSS

SEQ ID NO: 326 - protein, 108 aa, Artificial Sequence
  Synthetic Construct
DIQLTQSPSSLSASVGDRVTITCRASQTVRGTYLAWYQQKPGKAPKLLIYDASSLESGVPSRFSGSGSGTDFTLTISSLQPEDFATYYCQQYSSLWTFGQGTKVEIKR

SEQ ID NO: 327 - protein, 120 aa, Artificial Sequence
  Synthetic Construct
EVQLVESGGGLVQPGGSLRLSCAASGFSLSTGGVGVAWIRQAPGKGLEWLALIDWAGDKYYSPSLKSRLTISRDNSKNTLYLQLNSLRAEDTAVYYCARYGGSSYALDYWGQGTLVTVSS

SEQ ID NO: 328 - protein, 108 aa, Artificial Sequence; identical to SEQ ID NO: 264
  Synthetic Construct

SEQ ID NO: 329 - protein, 120 aa, Artificial Sequence
  Synthetic Construct
EVQLVESGGGLVQPGGSLRLSCAASGFSLNTAGVGVSWIRQAPGKGLEWLALIDWAGYKYYSPSLKSRLTISRDNSKNTLYLQLNSLRAEDTAVYYCARYGYRNYAMDYWGQGTLVTVSS

SEQ ID NO: 330 - protein, 108 aa, Artificial Sequence
  Synthetic Construct
DIQLTQSPSSLSASVGDRVTITCRASQSVRGNYLAWYQQKPGKAPKLLIYAASSLQSGVPSRFSGSGSGTDFTLTISSLQPEDFATYYCQQYSSYWTFGQGTKVEIKR

SEQ ID NO: 331 - protein, 120 aa, Artificial Sequence
  Synthetic Construct
EVQLVESGGGLVQPGGSLRLSCAASGFSLTTSGVGVAWIRQAPGKGLEWIGLIDHAGYTYYSPSLKSRLTISRDNSKNTLYLQLNSLRAEDTAVYYCARYGYDSYAMDYWGQGTLVTVSS

SEQ ID NO: 332 - protein, 108 aa, Artificial Sequence; identical to SEQ ID NO: 278
  Synthetic Construct

SEQ ID NO: 333 - protein, 120 aa, Artificial Sequence
  Synthetic Construct
EVQLVESGGGLVQPGGSLRLSCAASGFSLTTSGVGVGWIRQAPGKGLEWIGLIDYAGYKYYSPSLKSRLTISRDNSKNTLYLQLNSLRAEDTAVYYCARYGYRGYALDYWGQGTLVTVSS

SEQ ID NO: 334 - protein, 108 aa, Artificial Sequence; identical to SEQ ID NO: 316
  Synthetic Construct

SEQ ID NO: 335 - protein, 120 aa, Artificial Sequence
  Synthetic Construct
EVQLVESGGGLVQPGGSLRLSCAASGFSLSTAGVGVAWIRQAPGKGLEWLALIDWAGDTYYSPSLKSRLTISRDNSKNTLYLQLNSLRAEDTAVYYCARYGVGSYALDYWGQGTLVTVSS

SEQ ID NO: 336 - protein, 108 aa, Artificial Sequence; identical to SEQ ID NO: 264
  Synthetic Construct

SEQ ID NO: 337 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 338 - protein, 13 aa, Artificial Sequence
  Synthetic Construct
FSLSTGGVGVGWI

SEQ ID NO: 339 - protein, 20 aa, Artificial Sequence
  Synthetic Construct
LALIDWADDKYYSPSLKSRL

SEQ ID NO: 340 - protein, 15 aa, Artificial Sequence
  Synthetic Construct
ARGGSDTVIGDWFAY

SEQ ID NO: 341 - protein, 11 aa, Artificial Sequence
  Synthetic Construct
RASQSIGSYLA

SEQ ID NO: 342 - protein, 9 aa, Artificial Sequence
  Synthetic Construct
DASNLETGV

SEQ ID NO: 343 - protein, 10 aa, Artificial Sequence
  Synthetic Construct
YCQQGYYLWT

SEQ ID NO: 344 - protein, 123 aa, Artificial Sequence
  Synthetic Construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSS

SEQ ID NO: 345 - protein, 106 aa, Artificial Sequence
  Synthetic Construct
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIK

SEQ ID NO: 346 - protein, 11 aa, Artificial Sequence
  Synthetic Construct
  VARIANT 2: Xaa = Ser, Asn, or Thr
  VARIANT 4: Xaa = Ser, Ala, Gly, or Thr
  VARIANT 9: Xaa = Ser, Thr, Ala, or Gly
LXTXGVGVXWI

SEQ ID NO: 347 - protein, 20 aa, Artificial Sequence
  Synthetic Construct
  VARIANT 1: Xaa = Leu or Ile
  VARIANT 2: Xaa = Ala or Gly
  VARIANT 3: Xaa = Leu, Val, or Ile
  VARIANT 6: Xaa = Trp, His, or Tyr
  VARIANT 7: Xaa = Ala or Ser
  VARIANT 8: Xaa = Gly or Asp
  VARIANT 9: Xaa = Asp, Tyr, Ala, or Ser
  VARIANT 10: Xaa = Lys or Thr
  VARIANT 13: Xaa = Ser or Asn
  VARIANT 16: Xaa = Leu or Pro
  VARIANT 19: Xaa = Arg or His
XXXIDXXXXXYYXPSXKSXL

SEQ ID NO: 348 - protein, 12 aa, Artificial Sequence
  Synthetic Construct
  VARIANT 5: Xaa = Tyr, Val, Trp, Leu, or Gly
  VARIANT 6: Xaa = Ser, Gly, Arg, Asp, or Asn
  VARIANT 7: Xaa = Ser, Gly, or Asn
  VARIANT 10: Xaa = Ile, Leu, or Met
ARYGXXXYAXDY

SEQ ID NO: 349 - protein, 12 aa, Artificial Sequence
  Synthetic Construct
  VARIANT 2: Xaa = Ala or Thr
  VARIANT 5: Xaa = Ser, Thr, or Gly
  VARIANT 6: Xaa = Val or Ile
  VARIANT 7: Xaa = Arg, Ser, or His
  VARIANT 8: Xaa = Gly or Asn
  VARIANT 9: Xaa = Ser, Asn, or Thr
  VARIANT 10: Xaa = Tyr or Phe
RXSQXXXXXXLA

SEQ ID NO: 350 - protein, 9 aa, Artificial Sequence
  Synthetic Construct
  VARIANT 1: Xaa = Ala or Asp
  VARIANT 3: Xaa = Ser or Phe
  VARIANT 4: Xaa = Thr or Ser
  VARIANT 6: Xaa = Gln or Glu
XAXXLXSGV

SEQ ID NO: 351 - protein, 10 aa, Artificial Sequence
  Synthetic Construct
  VARIANT 6: Xaa = Ser, Gly, or Asn
  VARIANT 8: Xaa = Leu, Tyr, Ser, Trp or Phe
YCQQYXSXWT